(19) United States
(12) Patent Application Publication
(10) Pub. No.: US 2009/0221030 A1
(43) Pub. Date: Sep. 3, 2009

(54) SIGNAL SEQUENCES AND CO-EXPRESSED CHAPERONES FOR IMPROVING PROTEIN PRODUCTION IN A HOST CELL

(76) Inventors: Kai Bao, Palo Alto, CA (US); Huaming Wang, Fremont, CA (US)

Correspondence Address: STEVEN G. BACSI;Danisco US Inc. Genencor Division, 925 Page Mill Road, Palo Alto, CA, 94304-1013, US

(21) Appl. No.: 12/261,306

(22) Filed: Oct. 30, 2008

Related U.S. Application Data
(60) Provisional application No. 60/984,430, filed on Nov. 1, 2007.

(51) Int. Cl. C12P 21/00

(52) U.S. Cl. 435/69.1

(57) ABSTRACT

The invention provides methods and compositions for improved protein production. The method comprises the steps of: (a) introducing into a host cell a first nucleic acid sequence comprising a signal sequence operably linked to a desired protein sequence; (b) expressing the first nucleic acid sequence; (c) co-expressing a second nucleic acid sequence encoding a chaperone or foldase selected from the group consisting of bip1, ero1, pdi1, tig1, prp1, ppi1, ppi2, prp3, prp4, calnexin, and lhs1; and (d) collecting the desired protein secreted from the host cell. The first nucleic acid sequence optionally comprises an enzyme sequence between the signal sequence and the desired protein sequence.

This application claims the benefit of U.S. Provisional Application No. 60/984,430, filed Nov. 1, 2007; which is incorporated herein by reference in its entirety.

### REFERENCE TO ELECTRONIC SEQUENCE LISTING FILE

This application includes a sequence listing submitted electronically herewith as an ASCII text file named “sequence.txt”, which is 208 kB in size and was created Oct. 29, 2008; the electronic sequence listing is incorporated herein by reference in its entirety.

### FIELD OF THE INVENTION

This invention provides methods and compositions for improved protein production. In some embodiments, the methods provided herein involve the use of a signal sequence operably linked to a protein. In some embodiments, the signal sequence operably linked to a protein is expressed in combination with at least one chaperone in a host cell. In some embodiments, the protein is expressed in a filamentous fungal cell. In further embodiments, the methods of the present invention involve fusion of a protein to the catalytic domain of an enzyme, such as a glucoamylase or a CBH1. Some embodiments provide combinations of a signal sequence, one or more of a chaperone, chaperonin, and/or foldase, and/or fusion of the protein to a catalytic protein or domain.

### BACKGROUND OF THE INVENTION

Host cells such as yeast, filamentous fungi and bacteria have long been used to express and secrete foreign protein. Typically, production of these foreign or proteins in yeast, filamentous fungi and bacteria involves the expression and partial or complete purification of the protein from the host cell or the culture medium in which the cells are grown. While some proteins require purification from the intracellular milieu of the host cells, purification can be greatly simplified if the proteins are secreted from the cell into the culture media.

Extracellular protein secretion is a complicated and important aspect of protein production in various cell expression systems. One of the factors associated with protein secretion is proper protein folding. Many proteins can be reversibly unfolded and refolded in vitro at dilute concentrations, as all of the information required to specify a compact folded protein structure is present in the amino acid sequence of proteins. However, protein folding in vivo occurs in a concentrated milieu of numerous proteins in which intermolecular aggregation reactions compete with the intramolecular folding process. These complications are more significant in eukaryotic expression systems than in prokaryotic systems.

The first step in the eukaryotic secretory pathway is translocation of the nascent polypeptide across the endoplasmic reticulum (ER) membrane in extended form. Correct folding and assembly of a polypeptide occurs in the ER through the secretory pathway. However, in many cases, although the proteins are greatly overexpressed, they are poorly secreted. Indeed, in many cases the secretion signals that should facilitate such expression do not appear to accomplish this. The expression of desired proteins is further complicated by the interaction of other proteins. These factors are even more significant when expression of a protein obtained from one species, genus or family of organisms is attempted in another species, genus or family. For example, Basidiomycetes proteins (e.g., laccase) typically express poorly in Ascomycetes hosts such as Trichoderma. Indeed, despite much work in the area of fungal expression systems, there remains a need for improved extracellular expression of desired proteins.

### SUMMARY OF THE INVENTION

The invention provides methods and compositions for improved protein production. The methods involve the use of a signal sequence operably linked to a desired protein, which is expressed in combination with at least one chaperone in a host cell. In some embodiments, the protein is expressed in a filamentous fungal cell. In further embodiments, the methods of the present invention involve fusion of a desired protein to the catalytic domain of a host protein, such as a glucoamylase or a CBH1.

In some embodiments, the present invention provides methods and compositions to increase the production of proteins in filamentous fungal hosts (e.g., Ascomycetes), through the use of a secretory signal in combination with expression of a chaperone protein obtained from the same organism as the protein. In some embodiments, the protein is a non-Ascomycete protein that is fused to the secretory signal from an Ascomycetes host protein. In some additional embodiments, at least one chaperone protein finds use in increasing the expression of proteins fused to the catalytic domain of an Ascomycetes protein.

Some embodiments provide methods for producing at least one protein in an Ascomycetes host cell, by introducing into a host cell a polynucleotide comprising a desired protein operably linked to signal sequence from the same phylum, genus and/or species as the host; co-expressing a chaperone from the same phylum, genus and/or species as the protein; culturing the host cell under suitable culture conditions for the expression and production of the protein; and producing the protein. The method optionally includes recovering the produced protein. Some embodiments include fusing the protein to the catalytic domain of an enzyme from Ascomycetes. Other embodiments include fusing the protein to a full-length enzyme from Ascomycetes. In some embodiments, the Ascomycetes host cell is Trichoderma. In some embodiments, the chaperone is at least one of the following, BIP1, ERO1, PDI1, TIG1, PRP1, PPI1, PPI2, PRP3, PRP4, CALNEXIN, and LHS1.

The choice of protein is not limiting, and can include any of the following proteins from any genus, species, and/or family: laccases, glucoamylases, alpha amylases, granular starch hydrolyzing enzymes, cellulases, lipases, xylanases, cutinases, hemicellulases, proteases, oxidases, laccases and combinations thereof. Some embodiments include signal sequences from NSP24 or CBH1 genes. In some embodiments, the chaperone gene is bip1. Embodiments of the method can also include an Ascomycetes promoter. In some embodiments, the host cell and the signal sequence is from the same Ascomycetes host. In some embodiments, the promoter is the CBH1 promoter form Trichoderma. In some embodiments, the protein is a Basidiomycetes protein. In some embodiments, the host cell is an Ascomycetes host cell. In some embodiments, the host cell is a Basidiomycetes host cell and the protein is an Ascomycetes protein.

Some further embodiments provide methods for producing at least one protein in an Ascomycetes host cell, by introducing into an Ascomycetes host cell a polynucleotide comprising a desired protein fused to the catalytic domain of an enzyme from Ascomycetes, wherein the desired protein is a Basidiomycetes protein; co-expressing an Ascomycetes chaperone; culturing the Ascomycetes host cell under suitable culture conditions for the expression and production of the protein; and producing the protein. In some embodiments, the produced protein is recovered. In some embodiments, the protein is operably linked to an Ascomycetes signal sequence.

### BRIEF DESCRIPTION OF THE DRAWINGS

FIG. 1 shows the schematic of the Trichoderma expression plasmid pTrex4-laccaseD opt. The polynucleotide sequence is shown as SEQ ID NO: 1.

FIG. 2 shows the schematic of the Trichoderma expression plasmid pTrex2g-Bip1. The polynucleotide sequence is shown as SEQ ID NO: 2.

FIG. 3 shows the schematic of the Trichoderma expression plasmid pTrex2g-Pd1. The polynucleotide sequence is shown as SEQ ID NO: 3.

FIG. 4 shows the schematic of the Ero1 sequence used in the Trichoderma expression plasmid pTrex2g-Ero1. The polynucleotide sequence is shown as SEQ ID NO: 4.

FIG. 5 shows the schematic of the Trichoderma expression plasmid pTrGA-laccaseD opt. The polynucleotide sequence is shown as SEQ ID NO: 5.

FIG. 6 shows the schematic of the Trichoderma expression plasmid pKB408. The polynucleotide sequence is shown as SEQ ID NO: 6.

FIG. 7 shows the schematic of the Trichoderma expression plasmid pKB410. The polynucleotide sequence is shown as SEQ ID NO: 7.

FIGS. 8-1 to 8-4 show the T. reesei NSP24 Open Reading frame (ORF) SEQ ID NO:8. The signal peptide is the first 20 amino acids (SEQ ID NO: 9).

FIGS. 9-1 and 9-2 show the T. reesei CBH1 ORF (SEQ ID NO: 10). The signal sequence begins at base pair 210 and ends at base pair 260 (SEQ ID NO: 11). The catalytic core begins at base pair 261 through base pair 1698 (SEQ ID NO: 12), including intron 1 (from base pair 671 to 737) and intron 2 (from base pair 1435 to 1497). The linker sequence begins at base pair 1699 and ends at base pair 1770 (SEQ ID NO: 13). The CBH1 protein sequence is shown as SEQ ID NO: 14.

FIG. 10 illustrates the improvement of laccase production by fusion of the gene encoding C. unicolor laccase to the full-length Trichoderma glucoamylase. Strain #8-2 is CBH1 laccase fusion. Strain 1066-9, 1066-13, and 1066-15 are TrGA laccase fusion.

FIG. 11 illustrates the improvement of laccase production by fusion of the gene encoding C. unicolor laccase to the CBH1 or NSP24 signal sequence in shake flasks. Y axis shows the laccase activity as units/ml. X axis shows the strains (CBH1 fusion alone, or with signal sequence).

FIG. 12 illustrates the improvement of laccase production by fusion of the gene encoding C. unicolor laccase to the CBH1 or NSP24 signal sequence in fermentors. Y axis shows the laccase activity as units/ml. X axis shows the fermentation time as hours.

FIG. 13 illustrates the improvement of laccase production provided by the CBH1 signal sequence plus BIP1 chaperone expression. Y axis shows the laccase activity as units/mil. X axis shows the fermentation time as hours.

FIG. 14 illustrates the improvement of laccase production by co-expression of chaperones with C. unicolor in shake flasks at 3, 4, and 5 days. Y axis shows the laccase activity as units/ml. X axis shows the strains (KB410-13, or with co-expression of bip).

FIG. 15 illustrates the improvement of laccase production by fusion of the gene encoding C. unicolor laccase to the CBH1 signal sequence, catalytic domain and linker and co-expression with Bip1, pdi1 or ero1 chaperone. Y axis shows the laccase activity as units/ml. X axis shows the strains.

### DETAILED DESCRIPTION OF THE INVENTION

Unless otherwise indicated, the practice of the present invention involves conventional techniques commonly used in molecular biology, protein engineering, recombinant DNA techniques, microbiology, cell biology, cell culture, transgenic biology, immunology, and protein purification, which are within the skill of the art. Such techniques are known to those of skill in the art and are described in numerous texts and reference works. All patents, patent applications, articles and publications mentioned herein, both supra and infra, are hereby expressly incorporated herein by reference.

### DEFINITIONS

The term “Ascomycetes” refers to a class of fungi belonging to the phylum Ascomycota. Members of this phylum are distinguished by the presence of asci (i.e., specialized sac-like cells that contain ascospores).

The term “Basidiomycetes” refers to a class of fungi belonging to the phylum Basidiomycota. Members of this phylum are characterized by the production of basidospores, (i.e., sexual spores that are located on external areas of specialized club-shaped end cells referred to as basidia).

“Protease” means a protein or polypeptide domain of a protein or polypeptide that has the ability to catalyze cleavage of peptide bonds at one or more of various positions of a protein backbone (e.g. E.C. 3.4). Proteases are obtainable from microorganisms (e.g. a fungi or bacteria), plants, and/or animals.

An “acid protease” refers to a protease having the ability to hydrolyze proteins under acidic conditions.

As used herein, the term “chaperone” or “molecular chaperones” facilitate protein folding by shielding unfolded regions from surrounding proteins and do not enhance the rate of protein folding. This can include proteins and their homologs that assist the folding and glycosylation of the secretory proteins in the endoplasmic reticulum (ER). Chaperones may be resident in the ER. Exemplary chaperones include Bip (GRP78), GRP94 and yeast Lhs1p and those help the secretory protein to fold by binding to exposed hydrophobic regions in the unfolded states and preventing unfavorable interactions. Chaperones also include proteins that are involved in translocation of proteins through the ER membrane.

As used herein, “chaperonins” are proteins that assist protein folding to the native state (active state) utilizing ATP. Often the protein subunits are assembled together to form a large ring assemblies. For example, chaperonins act by binding normative proteins in their central cavities and then, upon binding ATP, release the substrate protein into a now-encapsulated cavity to fold productively.

“Foldase proteins” means proteins that catalyze steps in protein folding to increase the rate of protein folding. For example, they can assist in formation of disulphide bridges and formation of the right conformation of peptide chains adjacent to proline residues. Exemplary foldases include protein disulphide isomerase (pdi) and its homologs and prolyl-peptidyl cis-trans isomerase and its homologs.

As used herein, “NSP24 family protease” means an enzyme having protease activity in its native or wild type form that belonging to the family of NSP24 proteases. NSP24 proteases are acid proteases, such as acid fungal proteases. The NSP24 proteases have at least 85%, at least 90%, at least 93%, at least 95%, at least 96%, at least 97%, at least 98% and at least 99% sequence identity to the amino acid sequence of SEQ ID NO: 8 and biologically active fragments thereof.

As used herein, the term “a desired protein” means a protein of interest. A desired protein and a protein of interest are used interchangeably in this application. In some embodiments, the desired protein is a commercially important industrial protein. It is intended that the term encompass proteins that are encoded by naturally occurring genes, mutated genes and/or synthetic genes. The desired protein can be a protein native to the host cell, or non-native (heterologous) to the host cell.

As used herein, “derivative” means a protein which is derived from a precursor or parent protein (e.g., the native protein) by addition of one or more amino acids to either or both the C- and N-terminal end(s), substitution of one or more amino acids at one or a number of different sites in the amino acid sequence, deletion of one or more amino acids at either or both ends of the protein or at one or more sites in the amino acid sequence, or insertion of one or more amino acids at one or more sites in the amino acid sequence.

The term “recombinant” refers to a polynucleotide or polypeptide that does not naturally occur in a host cell. A recombinant molecule may contain two or more naturally occurring sequences that are linked together in a way that does not occur naturally.

The terms “peptides,” “proteins,” and “polypeptides” are used interchangeably herein.

As used herein, “percent (%) sequence identity” with respect to amino acid or nucleotide sequences is defined as the percentage of amino acid residues or nucleotides in a candidate sequence that are identical with the amino acid residues or nucleotides in a sequence of interest (e.g. a NSP24 signal peptide sequence), after aligning the sequences and introducing gaps, if necessary, to achieve the maximum percent sequence identity, and not considering any conservative substitutions as part of the sequence identity.

As used herein, the term “alpha-amylase (e.g., E.C. class 3.2.1.1)” refers to enzymes that catalyze the hydrolysis of alpha-1,4-glucosidic linkages. These enzymes have also been described as those effecting the exo or endohydrolysis of 1,4-α-D-glucosidic linkages in polysaccharides containing 1,4-α-linked D-glucose units. Another term used to describe these enzymes is “glycogenase.” Exemplary enzymes include alpha-1,4-glucan 4-glucanohydrase glucanohydrolase.

As used herein, the term “glucoamylase” refers to the amyloglucosidase class of enzymes (e.g., EC.3.2.1.3, glucoamylase, 1,4-alpha-D-glucan glucohydrolase). These are exo-acting enzymes, which release glucosyl residues from the non-reducing ends of amylose and amylopectin molecules. The enzyme also hydrolyzes alpha-1,6 and alpha-1,3 linkages although at much slower rate than alpha-1,4 linkages.

The term “promoter” means a regulatory sequence involved in binding RNA polymerase to initiate transcription of a gene.

A “heterologous promoter” as used herein refers to a promoter that has been placed in association with a gene or purified nucleic acid, but which is not naturally associated with that gene or purified nucleic acid.

A “purified preparation” and “substantially pure preparation” of a polypeptide, as used herein, mean a polypeptide that has been separated from cells, other proteins, lipids or nucleic acids with which it naturally occurs.

“Homologous,” as used herein, refers to the sequence similarity between two or more polypeptide molecules or between two or more nucleic acid molecules. When a position in the sequences being compared is occupied by the same base or amino acid monomer subunit, (e.g., if a position in each of two DNA molecules is occupied by adenine), then the molecules are homologous at that position. The percent of homology between two sequences is a function of the number of matching or homologous positions shared by the two sequences divided by the number of positions compared×100. For example, if 6 of 10, of the positions in two sequences are matched or homologous then the two sequences are 60% homologous. By way of example, the DNA sequences ATTGCC and TATGGC share 50% homology. Generally, a comparison is made when two sequences are aligned to give maximum homology. The term “% homology” is used interchangeably herein with the term “% identity” herein and refers to the level of nucleic acid or amino acid sequence identity between the nucleic acid sequences or amino acid sequences, when aligned using a sequence alignment program.

As used herein, the term “vector” refers to a polynucleotide sequence designed to introduce nucleic acids into one or more cell types. Vectors include cloning vectors, expression vectors, shuttle vectors, plasmids, phage particles, cassettes and the like.

As used herein, “expression vector” means a DNA construct including a DNA sequence which is operably linked to a suitable control sequence capable of affecting the expression of the DNA in a suitable host.

The term “expression” means the process by which a polypeptide is produced based on the nucleic acid sequence of a gene.

The term “co-expression” means that at least two different genes are expressed in one cell. They can be exogenous genes, or endogenous genes. They can be integrated or expressed from the same or different plasmids, and they can be expressed from the same or different promoter.

As used herein, “operably linked” means that a regulatory region, such as a promoter, terminator, secretion signal or enhancer region is attached to or linked to a structural gene and controls the expression of that gene. A signal sequence is operably linked to a protein if it directs the protein through the secretion system of a host cell.

As used herein, “microorganism” refers to a bacterium, a fungus, a virus, a protozoan, and other microbes or microscopic organisms.

The term “filamentous fungi” refers to all filamentous forms of the subdivision Eumycotina, as known in the art. These fungi are characterized by a vegetative mycelium with a cell wall composed of chitin, cellulose, and other complex polysaccharides. The filamentous fungi of the present invention are morphologically, physiologically, and genetically distinct from yeasts. Vegetative growth by filamentous fungi is by hyphal elongation and carbon catabolism is obligatory aerobic.

As used herein, the term “Trichoderma” and “Trichoderma sp.” refer to any fungal genus previously or currently classified as Trichoderma.

As used herein the term “culturing” refers to growing a population of microbial cells under suitable conditions in a liquid, semi-solid or solid medium. In some embodiments, culturing is conducted in a vessel or reactor, as known in the art. In some embodiments, culturing results in the fermentative bioconversion of a starch substrate, such as a substrate comprising granular starch, to an end-product.

“Fermentation” refers to the enzymatic and anaerobic breakdown of organic substances by microorganisms to produce simpler organic compounds. While fermentation often occurs under anaerobic conditions, it is not intended that the term be solely limited to strict anaerobic conditions, as fermentation also occurs in the presence of oxygen.

The term “introduced” in the context of inserting a nucleic acid sequence into a cell, means “transfection,” “transformation” or “transduction,” and includes reference to the incorporation of a nucleic acid sequence into a eukaryotic or prokaryotic cell wherein the nucleic acid sequence is either incorporated into the genome of the cell (e.g., chromosome, plasmid, plastid, or mitochondrial DNA), converted into an autonomous replicon, or transiently expressed (e.g., transfected mRNA).

As used herein, the terms “transformed,” “stably transformed” and “transgenic” used in reference to a cell means the cell has a non-native nucleic acid sequence integrated into its genome or as an episomal plasmid that is maintained through multiple generations.

As used herein, the term “heterologous” used in reference to a polypeptide or a polynucleotide encoding a desired protein means a polypeptide or polynucleotide that does not naturally occur in a host cell.

The term “homologous” or “endogenous” with reference to a polypeptide or a polynucleotide encoding a desired protein refers to a polypeptide or a polynucleotide that occurs naturally in or is naturally expressed by the host cell.

The term “overexpression” means the process of expressing a polypeptide in a host cell at a level that is greater than that produced by a wild-type host cell. In some embodiments, at least one polynucleotide is introduced into the host cell. In some further embodiments, the term refers to the expression of a homologous polypeptide at a concentration that is greater than that expression of the same homologous polypeptide expressed by a wild-type cell.

As described herein, one aspect of the invention features a “substantially pure” nucleic acid that comprises a nucleotide sequence encoding an NSP24 signal peptide or CBH1 signal peptide operably linked to a protein, and/or equivalents of such nucleic acids. In these embodiments, the nucleic acid is isolated from other nucleic acids and/or cell constituents.

The term “equivalent” refers to nucleotide sequences encoding functionally equivalent polypeptides. Equivalent nucleotide sequences encompass sequences that differ by one or more nucleotide substitutions, additions and/or deletions, such as allelic variants. For example in some embodiments, due to the degeneracy of the genetic code equivalent nucleotide sequences include sequences that differ from the nucleotide sequence of SEQ ID NO: 8, but that result in the production of polypeptides that are functionally equivalent to the polypeptide sequence encoded by SEQ ID NO:8.

This invention provides a method for producing a desired protein. The method comprises the steps of: (a) introducing into a host cell a first nucleic acid sequence comprising a signal sequence operably linked to a desired protein sequence; (b) expressing the first nucleic acid sequence; (c) co-expressing a second nucleic acid sequence encoding a chaperone or foldase selected from the group consisting of bip1, ero1, pdi1, tig1, prp1, ppi1, ppi2, prp3, prp4, calnexin, and lhs1; and (d) collecting the desired protein secreted from the host cell.

In one embodiment, the first nucleic acid sequence further comprises an enzyme sequence between the signal sequence and the desired protein sequence. For example, the enzyme sequence is obtained from a glucoamylase or from a CBH1 enzyme. In one embodiment, the enzyme sequence is a full-length enzyme sequence comprising a catalytic domain, a linker, and a binding domain. In another embodiment, the enzyme sequence comprises a catalytic domain sequence, which is linked to the desired protein sequence by a linker. In some embodiments, the enzyme is a host protein that is highly expressed and/or secreted in its natural host.

The first nucleic acid sequence further comprises a promoter upstream to a signal sequence. In one embodiment, the promoter is native to the host cell and is not naturally associated with the desired protein sequence.

The second nucleic acid sequence is operably linked to a promoter. In one embodiment, the promoter is native to the host cell and is not naturally associated with the second nucleic acid sequence.

### Increased Expression of Proteins

The present invention provides a method for the production of a desired protein in a host cell. The protein production is increased by inclusion of a secretory signal (e.g. NSP24 signal peptide or CBH1 signal peptide) in combination with co-expression of a chaperone, chaperonin, and/or foldase protein. In some embodiments, the secretory signal is from an Ascomycetes host protein. In some embodiment, the desired protein is fused to the catalytic domain of an enzyme.

The present invention provides significant advantages, especially in view of the fact that it can be difficult to produce large amounts of proteins from other fungi families in Ascomycete hosts. Indeed, those skilled in the art know that it is often difficult to produce any heterologous fungal protein in fungal or bacterial hosts. The present invention provides methods and compositions suitable for the production of any suitable protein in a suitable fungal or bacterial host. In some embodiments, the fungal host is an Ascomycetes and the protein is a Basidiomycetes protein, while in other embodiments, the fungal host is a Basidiomycetes and the protein is an Ascomycetes protein.

In some embodiments, the present invention provides methods for increasing expression and/or secretion of a protein in a host using a host signal peptide in combination with co-expression of one or more chaperones or foldases from the same organism as the source of the protein. Thus, in some embodiments, a heterologous Ascomycetes protein is expressed in a Basidiomycetes host using a Basidiomycetes host signal peptide and an Ascomycetes chaperone. In some alternative embodiments, a heterologous Basidiomycetes protein is expressed in an Ascomycetes host using an Ascomycetes signal peptide and an Ascomycetes or Basidiomycetes chaperone. In some embodiments, the Ascomycetes host is a member of the Trichoderma genus. In some embodiments, the Trichoderma is Trichoderma reesei, including various strains of T. reesei. In some alternative embodiments, the Basidiomycetes is a member of the genus Cerrena, including but not limited to C. unicolor.

In some embodiments of the present invention, expression and/or secretion of a desire protein is increased by fusing the protein to a host enzyme in combination with exogenous co-expression of one or more chaperones from the same organism as the desired protein. Co-expression is accomplished either via the same plasmid, or via separate plasmids.

In yet additional embodiments, expression and/or secretion of a desired protein is increased by linking the protein to a the catalytic domain of a host enzyme, in combination with operably linking the protein to a host signal sequence, and exogenous co-expression of one or more chaperones, chaperoning, and/or foldases, preferably from the same organism as the protein.

It is contemplated that elements recited in various embodiments provided herein will find use in any suitable combination. Thus, it is not intended that the embodiments be limited to the specific recitations provided herein, as aspects of the various embodiments find use in combination with each other.

### Signal Peptides

The specific signal peptide used in the present invention is not critical, as long as the signal peptide is operable in the host. An “operable signal peptide” is provided when the signal peptide increases secretion of a protein when operably linked to the protein in a host cell. In some embodiments, the signal peptide is obtained from a strongly secreted protein and/or is a strong signal peptide. A “strong signal peptide” results when the natural protein is strongly secreted by its natural host. In some embodiments, the signal peptide is obtained from an organism within the same phylum as the host cell. Indeed, in some embodiments, this is advantageous. In some embodiments, the signal peptide and the host cell are of the same genus, while in some additional embodiments, the signal peptide and the host cell are of the species. For example, in some embodiments, the host cell is an Ascomycetes host cell and the signal peptide is obtained from Ascomycetes. In some embodiments, the host cell is a Trichoderma and the signal peptide is from a Trichoderma. In some embodiments, the host cell is T. reesei and the signal peptide is obtained from T. reesei. In some embodiments, the signal peptide is a strong signal peptide. In some alternative embodiments, the host cell is a Basidiomycetes host cell and the signal peptide is obtained from Basidiomycetes. Some examples of signal peptides that find use in the present invention include, but are not limited to CBH1 and NSP24 signal peptides. While the signal peptides can work in other members of a phylum such as Ascomycetes, in some embodiments, signal peptides find optimum use when used in the genus from which it was obtained (i.e., to provide strong secretion).

As used herein, a “strongly secreted protein” is any protein that forms a significant amount of the total protein secreted from the cell. The total protein secreted from the cell is also referred to as “extracellular protein.” For example, a strongly secreted protein includes at least about 2% of the extracellular protein, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 75%, at least about 80%, at least about 85%, at least about 90%, at least about 95%, or at least about 99%. In some embodiments, the strongly secreted protein comprises at least about 5% of the extracellular protein in the culture supernatant.

### CBHI Signal Peptides, Linkers, and Catalytic Domains

Trichoderma reesei produces several cellulase enzymes, including cellobiohydrolase I (CBHI), which are folded into two separate domains (i.e., catalytic and binding domains) that are separated by an extended linker region. Foreign polypeptides have been secreted in T. reesei as fusions with the catalytic domain plus linker region of CBHI (See e.g., Nyyssonen et al., Bio/Technol. 11:591-595 [1993]). T. longibrachiatem also produces a CBHI that finds use in fusions, as well as in the isolation of a signal peptide and/or a linker. Linkers find use in connecting a catalytic domain of an enzyme and the desired polypeptide. Any suitable linker finds use in the present invention, as long as it forms an extended, semi-rigid spacer between independently folded domains. Such linker regions are found in several proteins, especially hydrolases (e.g., bacterial and fungal cellulases and hemicellulases; See e.g., Libby et al., Protein Engineering, Design and Selection (1994) vol. 7, 1109-1114).

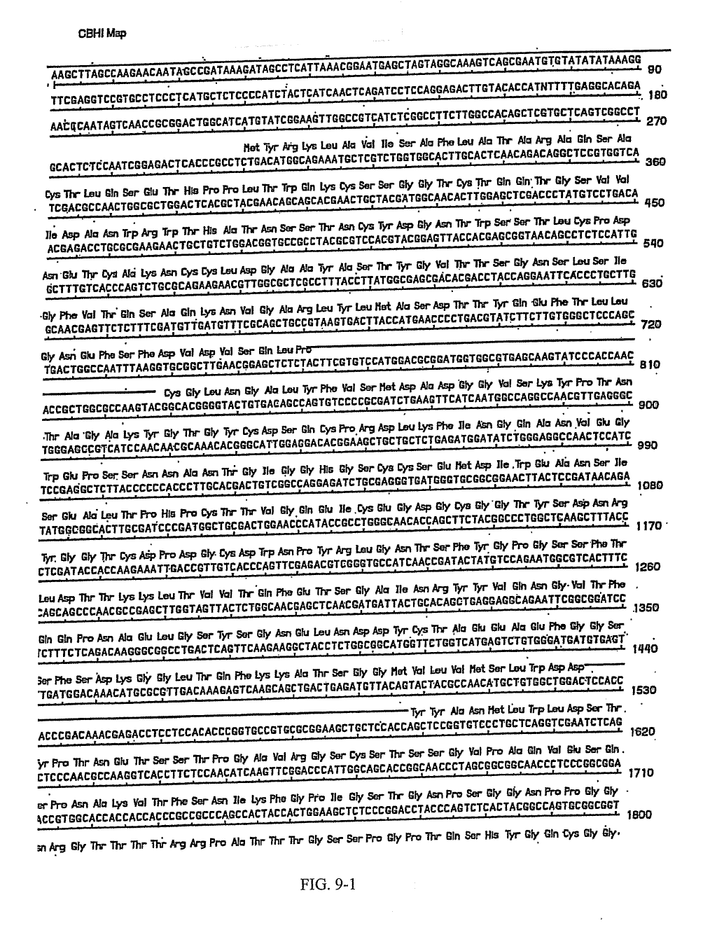

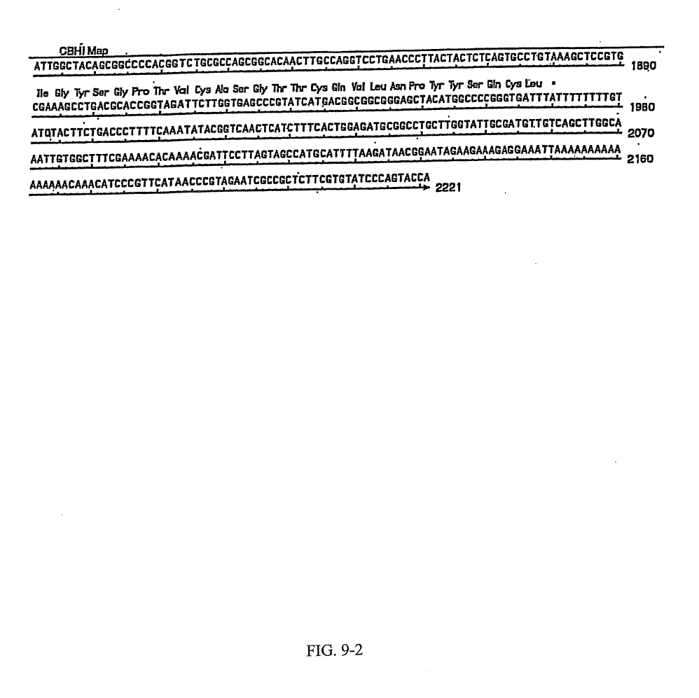

As shown in FIG. 9, for CBHI (SEQ ID NO: 10), the signal sequence begins at base pair 210 and ends at base pair 260 (SEQ ID NO: 11). The catalytic core begins at base pair 261 through base pair 1698 (SEQ ID NO: 12), including intron 1 (from base pair 671 to 737) and intron 2 (from base pair 1435 to 1497). The linker sequence begins at base pair 1699 and ends at base pair 1770 (SEQ ID NO: 13). The cellulose binding domain begins at base pair 1771 through base pair 1878. The sequence and domain information for CBHI can be found via the expasy organization website and is designated uniprot/P62694. CBHI homologs have been identified in a number of other Trichoderma species as well as other filamentous fungi and find use in the present invention as appropriate.

### NSP24 Signal Peptides and Polynucleotides

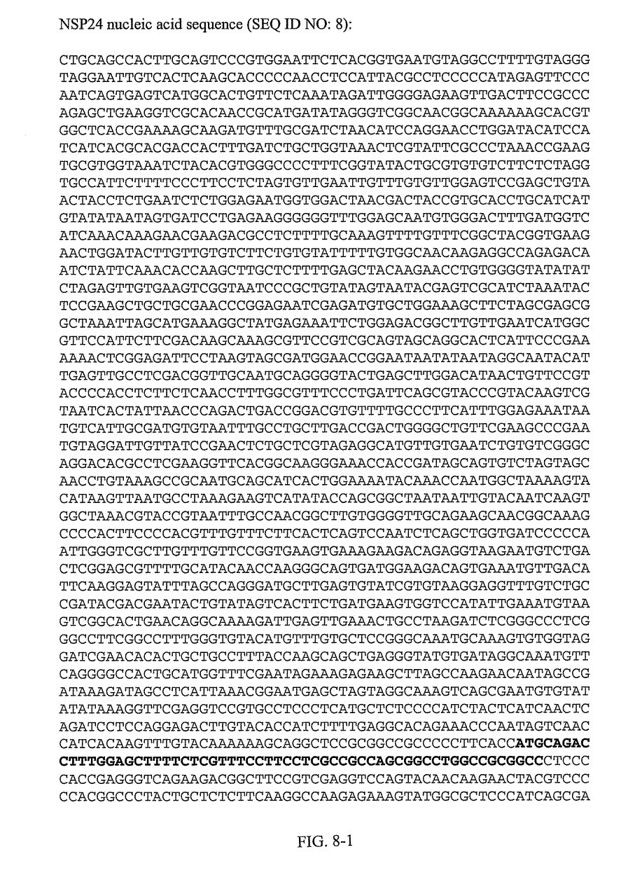

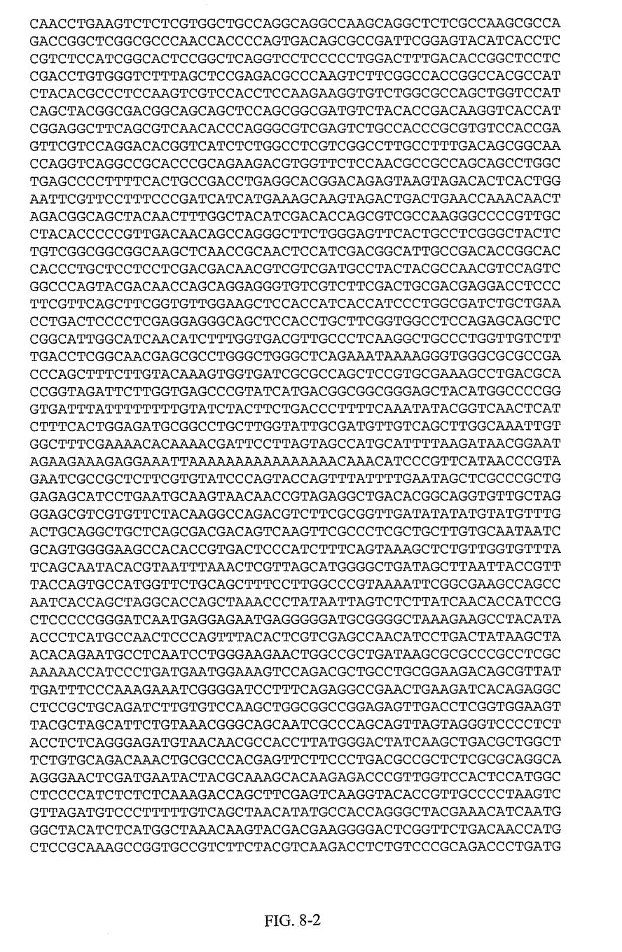

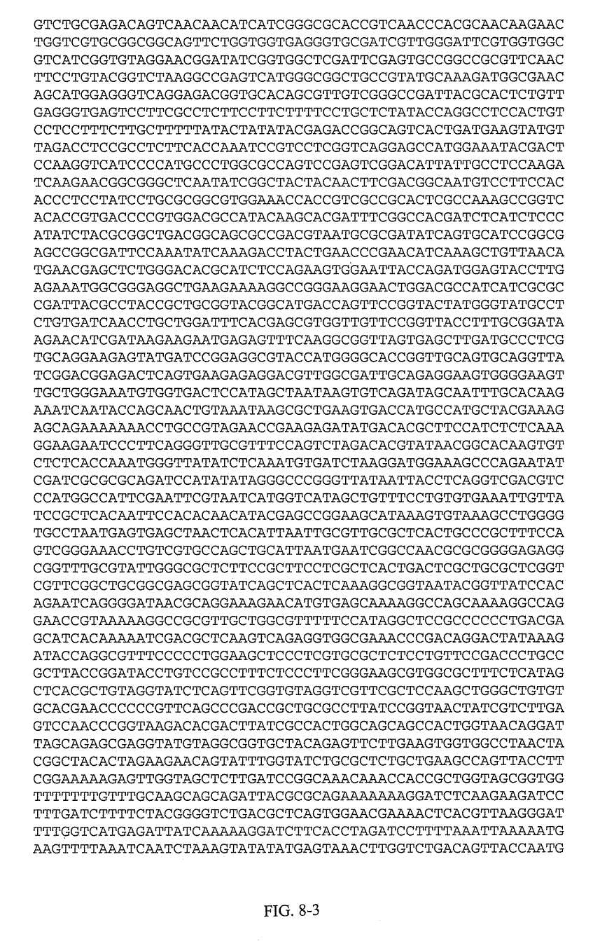

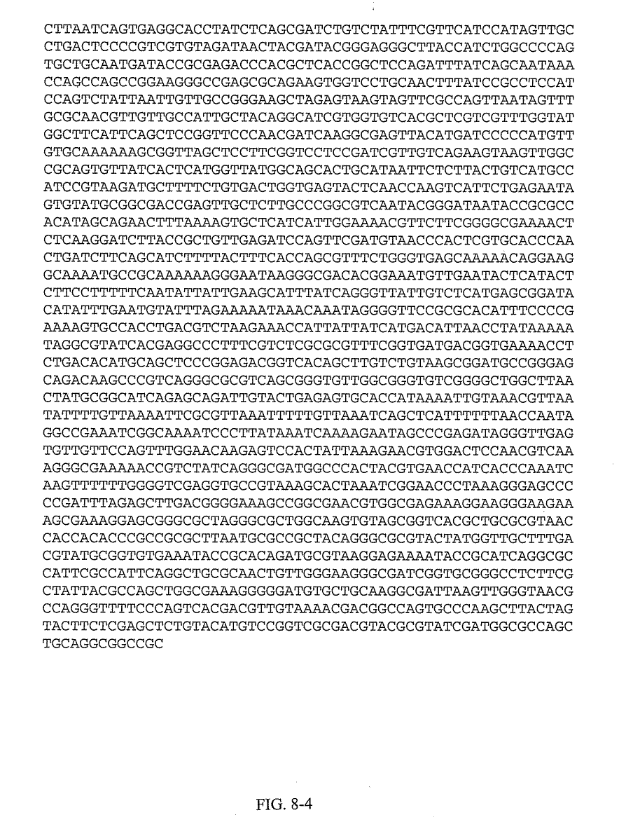

The NSP24 gene was isolated and sequenced from T. reesei (See e.g., U.S. Pat. No. 7,429,476, which is incorporated herein by reference in its entirety). Sequencing of this gene identified a sequence encoding a 407 amino acid open reading frame (SEQ ID NO: 8), as shown in FIG. 8. A signal peptide was identified as the first 20 amino acids (MQTFGAFLVSFLAASGLAAA; SEQ ID NO: 9) of SEQ ID NO: 8. NSP24 homologs have been identified in a number of other Trichoderma species as well as other filamentous fungi and find use in the present invention as appropriate. In some embodiments, the NSP24 signal sequence is used in an Ascomycetes organism. In some embodiments, the sequence is used in Trichoderma spp., and in some even more particularly embodiments, in T. reesei.

Thus, the present invention provides NSP24 family protease signal peptides that find use in secreting a protein. In some embodiments, the NSP24 signal peptide is designated “NSP24 aspartic protease signal peptide.”

### Polynucleotides of the Invention

The present invention provides various polynucleotides, including but not limited to polynucleotides encoding desired proteins, signal peptides, catalytic domains, linkers, chaperones, chaperonins and foldases. In some embodiments, polynucleotides comprise at least two of the above. In yet other embodiments, the polynucleotides of the present invention comprise at least three of the above.

In some embodiments, the polynucleotides encode proteins that comprise at least one amino acid substitution such as a “conservative amino acid substitution” using L-amino acids, wherein one amino acid is replaced by another biologically similar amino acid. Conservative amino acid substitutions are those that preserve the general charge, hydrophobicity/hydrophilicity, and/or steric bulk of the amino acid being substituted. Examples of conservative substitutions are those between the following groups: Gly/Ala, Val/Ile/Leu, Lys/Arg, Asn/Gln, Glu/Asp, Ser/Cys/Thr, and Phe/Trp/Tyr. In some embodiments, “derivative proteins” find use in the present invention. In some of these embodiments, the derivative proteins differ by as few as about 1 to about 10 amino acid residues, such as about 6 to about 10, as few as about 5, as few as about 4, about 3, about 2, or even 1 amino acid residue, compared to the “parent” protein sequence. Table 1 provides exemplary conservative amino acid substitutions recognized in the art. In additional embodiments, substitution involves one or more non-conservative amino acid substitutions, deletions, or insertions that do not abolish the signal peptide activity.

TABLE 1
Conservative Amino Acid Replacements

| | One | |
|---|---|---|
| For Amino | Letter | |
| Acid | Code | Replace with Any Of the Following |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, |
| | | cis-3,4, or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

In some embodiments, the polynucldeotides of the invention are native sequences. In some embodiments, the native sequences are isolated from nature, while in other embodiments they are produced by recombinant or synthetic means. The term “native sequence” specifically encompasses naturally-occurring truncated or secreted forms (e.g., biologically active fragments), and naturally-occurring variant forms of the native sequences.

Because of the degeneracy of the genetic code, more than one codon may be used to code for a particular amino acid. Therefore, in some embodiments, different DNA sequences are used to encode any of the polypeptides such as the signal peptide, the protein, the catalytic domain, and/or the chaperones. Indeed, it is intended that the present invention encompass different polynucleotide sequences that which encode the same polypeptide.

A nucleic acid is hybridizable to another nucleic acid sequence when a single stranded form of the nucleic acid can anneal to the other nucleic acid under appropriate conditions of temperature and solution ionic strength. Hybridization and washing conditions are well known in the art for hydridization under low, medium, high and very high stringency conditions. In general, hybridization involves a nucleotide probe and a homologous DNA sequence that form stable double stranded hybrids by extensive base-pairing of complementary polynucleotides. In some embodiments, the filter with the probe and homologous sequence are washed in 2× sodium chloride/sodium citrate (SSC), 0.5% SDS at about 60° C. (medium stringency), 65° C. (medium/high stringency), 70° C. (high stringency) and about 75° C. (very high stringency) (See e.g., Current Protocols in Molecular Biology, John Wiley & Sons, New York, 1989, 6.3.1-6.3.6, hereby incorporated by reference);

The present invention encompasses allelic variations, natural mutants, induced mutants, proteins encoded by DNA that hybridizes under high or low stringency conditions to a nucleic acid which encodes a laccase, a signal sequence of NSP24, a signal sequence of CBHI, catalytic domains, chaperones, chaperonins and foldases. Nucleic acids and polypeptides of the present invention include those that differ from the sequences disclosed herein by virtue of sequencing errors in the disclosed sequences.

“Homology of DNA sequences” is determined by the degree of identity between two DNA sequences. Homology or “percent identity” is often determined for polypeptide sequences and/or nucleotides sequences using computer programs. Methods for performing sequence alignment and determining sequence identity are well-known to the skilled artisan, may be performed without undue experimentation, and calculations of identity values are obtainable with definiteness. A number of algorithms are available and known to those of skill in the art, for aligning sequences and determining sequence identity. Computerized programs using these algorithms are also available and well-known to those in the art, including, but are not limited to: ALIGN or Megalign (DNASTAR) software, or WU-BLAST-2, GAP, BESTFIT, BLAST, FASTA, TFASTA, and CLUSTAL. Those skilled in the art know how to determine appropriate parameters for measuring alignment, including algorithms needed to achieve maximal alignment over the length of the sequences being compared. The sequence identity can be determined using the default parameters determined by the program. In some embodiments, sequence identity is determined by the Smith-Waterman homology search algorithm (Smith Waterman, Meth. Mol. Biol., 70:173-187 [1997)) as implemented in MSPRCH program (Oxford Molecular) using an affine gap search with the following search parameters: gap open penalty of 12, and gap extension penalty of 1. Paired amino acid comparisons can be carried out using the GAP program of the GCG sequence analysis software package of Genetics Computer Group, Inc. (Madison, Wis.), employing the blosum62 amino acid substitution matrix, with a gap weight of 12 and a length weight of 2. With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least about 20 contiguous amino acid residues, and may be about 30, about 40, about 50, or more amino acid residues. In some embodiments, corrections for increased sequence identity associated with inclusion of gaps in the derivative's amino acid sequence are made by assigning gap penalties.

In some embodiments, the protein, signal peptide, enzyme catalytic domain, chaperone, chaperonin, and/or foldase encompassed by the invention is derived from a bacterium or a fungus, such as a filamentous fungus. Exemplary filamentous fungi include Aspergillus spp. and Trichoderma spp. One exemplary Trichoderma spp. is T. reesei. However, in some embodiments, the signal peptide and/or DNA encoding the signal peptide provided by the present invention is derived from another genus or species of fungi, including but not limited to Absidia spp.; Acremonium spp; Agaricus spp; Anaeromyces spp; Aspergillus spp., including, but not limited to A. aculeatus, A. awamori, A. flavus, A. foetidus, A. fumaricus, A. fumigatus, A. nidulans, A. niger, A. oryzae, A. terreus and A. versicolor; Aeurobasidium spp.; Cerrena spp.; Cephalosporum spp.; Cephalosporium spp.; Chaetomium spp.; Coprinus spp.; Dactyllum spp.; Dactylium spp.; Fusarium spp., including F. conglomerans, F. decemcellulare, F. javanicum, F. lini, F. oxysporum and F. solani; Gliocladium spp.; Humicola spp., including H. insolens and H. lanuginosa; Mucor spp.; Neurospora spp., including N. crassa and N. sitophila; Neocallimastix spp.; Orpinomyces spp.; Penicillium spp; Phanerochaete spp.; Phlebia spp.; Piromyces spp.; Rhizopus spp.; Schizophyllum spp.; Stachybotrys spp.; Trametes spp.; Trichoderma spp., including T. reesei, T. reesei (longibrachiatum) and T. viride; and Zygorhynchus spp.

### Catalytic Domain Fusion

Fusing a desired protein to an enzyme often allows for increased expression and/or secretion of the desired protein. In general, the enzyme sequence is upstream to the desire protein sequence in the construct. For example, the enzyme is obtained from a glucoamylase or from a CBH1 enzyme. In one embodiment, the enzyme sequence is a full-length enzyme sequence comprising a catalytic domain, a linker, and a binding domain. In another embodiment, the enzyme sequence comprises a catalytic domain sequence, which is linked to the desired protein sequence by a linker or a portion of the linker. In some embodiments, the enzyme is a host protein that is highly expressed and/or secreted in its natural host. For example, when the host cell is a Trichoderma host cell, the enzyme is from a Trichoderma protein. However, it is to be understood that many filamentous fungal proteins find use in fusion to proteins and can be used in other filamentous fungal hosts with success.

### Chaperones, Chaperonins and Foldases

The specific chaperone, chaperonin, and/or foldase used in the methods and polynucleotides included in the invention is not critical. Further, when describing the uses of chaperone, chaperonin, and/or foldase herein, they are used interchangeably in a method. For example, when describing a method using a chaperone, it is to be understood that a foldase and/or chaperonin could be used in place of or in addition to the recited chaperone. Chaperone, chaperonin, and/or foldase suitable for this invention are those that are active in a host cell and act to increase expression of the desired protein.

In some embodiments, the chaperone, chaperonin, and/or foldase is from the same phylum of organisms as the protein, and can be from the same genus, and can also be from the same genus and species. In some embodiments, the chaperone, chaperonin, and/or foldase is from a Basidiomycete and the protein is a basiomycetes protein. In some embodiments, the chaperone, chaperonin, and/or foldase are used in combination. In some embodiments, fragments of chaperone, chaperonin, and/or foldase having substantially the same function as the full-length chaperone, chaperonin, and/or foldase can be used. Exemplary chaperone, chaperonin, and/or foldase include those disclosed in U.S. patent application 60/919,332 and WO 2008/115596, which are incorporated herein by reference in their entirety. Exemplary chaperone, chaperonin, and/or foldase include, but are not limited to: BIP1, CLX1, ERO1, LHS1, PRP3, PRP4, PRP1, TIG1, PDI1, PPI1, PPI2, SCJ1, ERV2, EDEM, and SIL1. Table 2 provides a number of the sequences for chaperone, chaperonin, and/or foldase usable in the invention.

TABLE 2
Exemplary Nucleic Acid and Polypeptide Sequences
of Secretion-Enhancing Proteins

| | Exemplary Nucleotide | Exemplary Polypeptide |
|---|---|---|
| Protein | Acid Sequence | Sequence |
| BIP1 | SEQ ID NO: 15 | SEQ ID NO: 30 |
| CLX1 | SEQ ID NO: 16 | SEQ ID NO: 31 |
| ERO1 | SEQ ID NO: 17 | SEQ ID NO: 32 |
| LHS1 | SEQ ID NO: 18 | SEQ ID NO: 33 |
| PRP3 | SEQ ID NO: 19 | SEQ ID NO: 34 |
| PRP4 | SEQ ID NO: 20 | SEQ ID NO: 35 |
| PRP1 | SEQ ID NO: 21 | SEQ ID NO: 36 |
| TIG1 | SEQ ID NO: 22 | SEQ ID NO: 37 |
| PDI1 | SEQ ID NO: 23 | SEQ ID NO: 38 |
| PPI1 | SEQ ID NO: 24 | SEQ ID NO: 39 |
| PPI2 | SEQ ID NO: 25 | SEQ ID NO: 40 |
| SCJ1 | SEQ ID NO: 26 | SEQ ID NO: 41 |
| ERV2 | SEQ ID NO: 27 | SEQ ID NO: 42 |
| EDEM | SEQ ID NO: 28 | SEQ ID NO: 43 |
| SIL1 | SEQ ID NO: 29 | SEQ ID NO: 44 |

### Molecular Biology—Promoters and Expression Vectors

The present invention utilizes routine techniques in the field of recombinant genetics, well-known to those of skill in the art. In some embodiments, the present invention provides heterologous genes comprising gene promoter sequences (e.g., from, filamentous fungi) that are typically cloned into intermediate vectors before transformation into host cells (e.g., Trichoderma reesei cells) for replication and/or expression. These intermediate vectors are typically prokaryotic vectors (e.g., plasmids, or shuttle vectors).

In general, the expression of a desired protein is accomplished under any suitable promoter. In one embodiment, a promoter non-native to a host is operably linked to a polynucleotide encoding a desired protein that is either native or non-native to a host. In another embodiment, a promoter native to a host is operably linked to a polynucleotide encoding a desired protein that is either native or non-native to a host. In some embodiments, the desired protein is expressed under a heterologous promoter, which is not naturally associated with the desired protein gene. While in some other embodiments, the desired protein is expressed under a constitutive or inducible promoter. In some embodiments, the desired protein is expressed in a Trichoderma expression system with a cellulase promoter (e.g., the cbh1 promoter).

As used herein, the term “promoter” refers to a nucleic acid sequence that functions to direct transcription of a downstream gene. A promoter can include necessary nucleic acid sequences near the start site of transcription, such as, in the case of a polymerase II type promoter, a TATA element. The promoter together with other transcriptional and translational regulatory nucleic acid sequences, collectively referred to as “regulatory sequences” controls the expression of a gene. In general, the regulatory sequences include, but are not limited to, promoter sequences, ribosomal binding sites, transcriptional start and stop sequences, translational start and stop sequences, and enhancer or activator sequences. The regulatory sequences are generally appropriate for and recognized by the host in which the downstream gene is being expressed. In some embodiments, the promoter used is from the same phylum as the host cell, and in other embodiment the promoter is from the same genus as the host cell, and in some embodiments from the same genus and species as the host cell.

A “constitutive promoter” is a promoter that is active under most environmental and developmental conditions. An “inducible” or “repressible promoter” is a promoter that is active under environmental or developmental regulation. In some embodiments, promoters are inducible or repressible due to changes in environmental factors including, but not limited to, carbon, nitrogen or other nutrient availability, temperature, pH, osmolarity, the presence of heavy metal(s), the concentration of inhibitor(s), stress, or a combination of the foregoing, as is known in the art. In some other embodiments, promoters are inducible or repressible by metabolic factors, such as the level of certain carbon sources, the level of certain energy sources, the level of certain catabolites, or a combination of the foregoing, as is known in the art.

Suitable non-limiting examples of promoters include cbh1, cbh2, egl1, egl2, egl3, egl4, egl5, xyn1, and xyn2, repressible acid phosphatase gene (phoA) promoter of P. chrysogenum (See, Graessle et al., Appl. Environ. Microbiol., 63:753-756 [1997]), glucose-repressible PCK1 promoter (See, Leuker et al., Gene 192:235-240 [1997]), maltose-inducible, glucose-repressible MRP1 promoter (See, Munro et al., Mol. Microbiol., 39 1414-1426 [2001]), methionine-repressible MET3 promoter (See, Liu et al., Eukary. Cell 5:638-649 [2006]), pKi promoter, and cpc1 promoter.

In some embodiments of the present invention, the promoter in the reporter gene construct is a temperature-sensitive promoter. In some embodiments, the activity of the temperature-sensitive promoter is repressed by elevated temperature. In some embodiments, the promoter is a catabolite-repressed promoter. In some embodiments, the promoter is repressed by changes in osmolarity. In some embodiments, the promoter is inducible or repressible by the levels of polysaccharides, disaccharides, or monosaccharides present in the culture medium.

An example of an inducible promoter that finds use in the present invention is the cbh1 promoter of T. reesei, the nucleotide sequence of which is deposited in GenBank under Accession Number D86235. Other exemplary promoters include promoters involved in the regulation of genes encoding cellulase enzymes, including, but not limited to, cbh2, egl1, egl2, egl3, egl5, xyn1 and xyn2.

In some embodiments of the present invention, in order to obtain high levels of expression of a cloned gene, the heterologous gene is advantageously positioned about the same distance from the promoter as in the naturally occurring gene. However, as is known in the art, some variation in this distance can be accommodated without loss of promoter function.

In some embodiments, a natural promoter modified by replacement, substitution, addition or elimination of one or more nucleotides finds use in the present invention, as long as the modifications do not change the function of the promoter. Indeed, it is intended that the present invention encompasses and is not constrained by such alterations to the promoter.

The expression vector/construct typically contains a transcription unit or expression cassette that contains all of the additional elements required for the expression of the heterologous sequence. Thus, a typical expression cassette contains a promoter operably linked to the heterologous nucleic acid sequence and signals required for efficient polyadenylation of the transcript, ribosome binding sites, and translation termination. Additional elements within the cassette may include enhancers and, if genomic DNA is used as the structural gene, introns with functional splice donor and acceptor sites, secretion leader peptides, leader sequences, linkers, and cleavage sites.

The practice of the present invention is not constrained by the choice of promoter in the genetic construct. As indicated above, exemplary promoters are the Trichoderma reesei cbh1, cbh2, eg1, eg2, eg3, eg5, xln1 and xln2 promoters. Additional promoters that find use in the present invention include those from A. awamori and A. niger glucoamylase genes (glaA) (See, Nunberg et al., Mol. Cell. Biol., 4:2306-2315 [1984]) and the promoter from A. nidulans acetamidase. An exemplary promoter for vectors used in Bacillus subtilis is the AprE promoter; an exemplary promoter used in E. coli is the Lac promoter, an exemplary promoter used in Saccharomyces cerevisiae is PGK1, an exemplary promoter used in Aspergillus niger is glaA, and an exemplary promoter for Trichoderma reesei is cbhI. However, it is not intended that the present invention be limited to these specific cells nor these specific promoters, as other cells and promoters find use in various embodiments.

In some embodiments, in addition to a promoter sequence, the expression cassette also contains a transcription termination region downstream of the structural gene to provide for efficient termination. In some embodiments, the termination region is obtained from the same gene as the promoter sequence, while in other embodiments, it is obtained from different genes.

Although any suitable functional fungal terminator finds use in the present invention, some exemplary terminators include, but are not limited to the terminator from Aspergillus nidulans trpC gene (See, Yelton et al., Proc. Natl. Acad. Sci. USA 81:1470-1474 (1984); Mullaney et al., (Molecular Genetics and Genomics [MGG] 199:37-45 (1985)), the Aspergillus awamori or Aspergillus niger glucoamylase genes (See, Nunberg et al., Mol. Cell. Biol., 4:2306 (1984); Boel et al., EMBO J., 3:1581-1585 (1984)), the Aspergillus oryzae TAKA amylase gene, the Mucor miehei carboxylprotease gene (EP Pat. Publ. No. 0 215 594) and the Trichoderma reesei CBH1 gene.

It is not intended that the expression vector used to transport the genetic information into the host cell be limited to any particular vector. It is contemplated that any of the conventional vectors used for expression in eukaryotic or prokaryotic cells will find use in the present invention. Standard bacterial expression vectors include, but are not limited to bacteriophages λ and M13, as well as plasmids such as pBR322-based plasmids, pSKF, pET23D, and fusion expression systems such as MBP, GST, and LacZ. In some embodiments, epitope tags are added to recombinant proteins to provide convenient methods of isolation (e.g., c-myc). Examples of suitable expression and/or integration vectors are well-known to those in the art (See e.g., Bennett and Lasure (eds.) More Gene Manipulations in Fungi, Academic Press pp. 70-76 and pp. 396-428 (1991); U.S. Pat. No. 5,874,276. Various commercial vendors (e.g., Promega, Invitrogen, etc.) provide useful vectors, as known to those of skill in the art. Some specific useful vectors include, but are not limited to pBR322, pUC18, pUC100, pDON™201, pENTR™, pGEN®3Z and pGEN®4Z. However, it is intended that the present invention encompass other expression vectors which serve equivalent functions and which are, or become, known in the art. Thus, a wide variety of host/expression vector combinations find use in expressing the DNA sequences of the present invention. In some embodiments, useful expression vectors comprise segments of chromosomal, non-chromosomal and/or synthetic DNA sequences (e.g., various known derivatives of SV40) and known bacterial plasmids (e.g., plasmids from E. coli including col E1, pCR1, pBR322, pMb9, pUC19, pSL1180 and their derivatives), wider host range plasmids (e.g., RP4), phage DNAs (e.g., the numerous derivatives of phage lambda., such as NM989, and other DNA phages, such as M13, and filamentous single stranded DNA phages), and yeast plasmids (e.g., the 2.mu plasmid or derivatives thereof).

In some embodiments, an expression vector includes a selectable marker. Examples of selectable markers include those that confer antimicrobial resistance. Nutritional markers also find use in the present invention, including those markers known in the art as amdS, argB and pyr4. Markers useful for the transformation of Trichoderma are known in the art (See e.g., Finkelstein, in Biotechnology of Filamentous Fungi, Finkelstein et al., (eds.), Butterworth-Heinemann, Boston Mass., chapter 6 (1992)). In some embodiments, the expression vectors also include a replicon, a gene encoding antibiotic resistance to permit selection of bacteria that harbor recombinant plasmids, and/or unique restriction sites in nonessential regions of the plasmid to allow insertion of heterologous sequences. It is intended that any suitable antibiotic resistance gene will find use in the present invention. In some embodiments in which T. reesei is the host cell, the prokaryotic sequences are preferably chosen such that they do not interfere with the replication or integration of the DNA in T. reesei.

In some embodiments, an expression vector includes a reporter gene alone or, optionally as a fusion with the protein of interest. Examples of reporter genes include but are not limited to, fluorescent reporters, color detectable reporters (e.g., β-galactosidase), and biotinylated reports. In some embodiments, when the reporter molecule is expressed, it is used to identify whether the signal peptide is active in a host cell. If the signal peptide is active, the reporter molecule is secreted from the cell. In some embodiments, the signal peptide is initially operably linked to the reporter, in order to identify secretion from a particular host cell. Alternative methods such as those using antibodies specific to the protein of interest and/or the signal peptide also find use in determining whether or not the protein of interest is secreted.

In some embodiments, the methods of transformation of the present invention result in the stable integration of all or part of the transformation vector into the genome of a host cell, such as a filamentous fungal host cell. However, transformation resulting in the maintenance of a self-replicating extra-chromosomal transformation vector is also contemplated.

Many standard transfection methods find use in the present invention to produce bacterial and filamentous fungal (e.g., Aspergillus or Trichoderma) cell lines that express large quantities of the proteins. Methods for the introduction of DNA constructs into cellulase-producing strains of Trichoderma are well-known to those of skill in the art (See e.g., Lorito et al., Curr. Genet., 24:349-356 [1993]; Goldman et al., Curr. Genet., 17:169-174 [1990]; Penttila et al., Gene 6: 155-164 [1987]; U.S. Pat. No. 6,022,725; U.S. Pat. No. 6,268,328; Nevalainen et al., “The Molecular Biology of Trichoderma and its Application to the Expression of Both Homologous and Heterologous Genes” in Molecular Industrial Mycology, Leong and Berka (eds.), Marcel Dekker Inc., NY [1992) pp 129-148; Yelton et al., Proc. Natl. Acad. Sci. USA 81: 1470-1474 [1984]; Bajar et al., Proc. Natl. Acad. Sci. USA 88: 8202-8212 [1991]; Fernandez-Abalos et al., Microbiol., 149:1623-1632 [2003); and Brigidi et al., FEMS Microbiol. Lett., 55:135-138 [1990]).

However, any of the well-known procedures for introducing foreign nucleotide sequences into host cells find use in the present invention. These methods include, but are not limited to the use of calcium phosphate transfection, polybrene, protoplast fusion, electroporation, biolistics, liposomes, microinjection, plasmid vectors, viral vectors and any of the other well known methods for introducing cloned genomic DNA, cDNA, synthetic DNA or other foreign genetic material into a host cell, as well-known to those of skill in the art. Also of use is the Agrobacterium-mediated transfection method (See e.g., U.S. Pat. No. 6,255,115). It is only necessary that the particular genetic engineering procedure used be capable of successfully introducing at least one gene into a host cell that is capable of expressing the gene. In some embodiments, the invention provides methods for producing a protein, comprising the steps of introducing into a host cell a polynucleotide comprising an NSP24 signal peptide linked to a nucleic acid encoding a protein, culturing the host cell under suitable culture conditions for the expression and production of the protein, and producing said protein. In some embodiments, the protein is secreted from the host cell. In some alternative embodiments, the present invention provides methods for producing a protein, comprising the steps of introducing into a host cell a polynucleotide comprising an CBH1 signal peptide operably linked to a nucleic acid encoding a protein, culturing the host cell under suitable culture conditions for the expression and production of the protein, and producing said protein. In some embodiments, the protein is secreted from the host cell.

After the expression vector is introduced into the host cells, the transfected or transformed cells are cultured under conditions favoring expression of genes under control of the gene promoter sequences. In some embodiments, large batches of transformed cells are cultured. In some embodiments, the product (i.e., the protein) is harvested from the cells and/or recovered from the culture using standard techniques.

Thus, the invention herein provides for the expression and enhanced secretion of desired polypeptides whose secretion is enhanced by signal peptide sequences, fusion DNA sequences, and various heterologous constructs as well as expression of chaperones, chaperonins and/or foldases. The invention also provides processes for expressing and secreting high levels of such desired polypeptides.

### Desired Proteins

The term “desired protein” means any protein of interest. The desired protein can be a protein native to a host cell, or non-native (heterologous) to a host cell. In some embodiments, the desired protein is a fungal protein. In some embodiments, the host is an Ascomycete host and the protein is any protein other than an Ascomycetes protein. In some embodiments, the host is a Basidiomycete host and the protein is any protein other than a Basidiomycete protein. In some embodiments, the protein is any protein other than a Trichoderma protein. In some other embodiments, the protein is any protein other than an Aspergillus protein.

It is not intended that the present invention be limited to any particular type of protein. Indeed, it is intended that the present invention encompass any protein of interest. Some non-limiting examples of desired proteins include, but are not limited to glucoamylases, alpha amylases, granular starch hydrolyzing enzymes, cellulases, lipases, xylanases, cutinases, hemicellulases, proteases, oxidases, laccases and combinations thereof.

In some embodiments, the glucoamylase is a wild type glucoamylase obtained from a filamentous fungal source, such as a strain of Aspergillus, Trichoderma or Rhizopus. However, in other embodiments, the glucoamylase is a protein engineered glucoamylase (e.g., a variant of an Aspergillus niger glucoamylase). In some other embodiments, compositions of the present invention also comprise at least one protease and at least one alpha amylase. In some embodiments, the alpha amylase is obtained from a bacterial source (e.g., Bacillus spp.), or from a fungal source (e.g., an Aspergillus spp.). In some embodiments, the compositions also include at least one protease, and/or at least one glucoamylase, and/or at least one alpha amylase enzymes. In some embodiments, the protein is laccase, such as laccase obtained from Basidiomycetes, and in some embodiments, from the genus Cerrena, such as C. unicolor. Commercial sources of these enzymes are known and available from, for example Genencor International, Inc. and Novozymes A/S.

### Laccase and Laccase Related Enzymes

In one preferred embodiment, laccases and laccase-related enzymes are desired proteins. It is not intended that the present invention be limited to any particular laccase, as any laccase enzyme within the enzyme classification (EC 1.10.3.2) is encompassed. In some embodiments, the laccase enzymes are obtained from microbial or plant origin. In some embodiments, the microbial laccase enzymes are derived from bacteria or fungi (including filamentous fungi and yeasts). Although it is not intended that the present invention be limited to specific laccases, suitable examples include laccases derivable from Aspergillus, Neurospora (e.g. N. crassa), Podospora, Botrytis, Collybia, Cerrena, Stachybotrys, Panus, (e.g., Panus rudis), Thieilava, Fomes, Lentinus, Pleurotus, Trametes (e.g., T. villosa and T. versicolor), Rhizoctonia (e.g. R. solani), Coprinus (e.g. C. plicatilis and C. cinereus), Psatyrella, Myceliophthora (e.g., M. thermonhila), Schytalidium, Phlebia (e.g. P. radita; See e.g., WO 92/01046), Coriolus (e.g. C. hirsutus; See e.g., JP 2-238885), Spongipellis, Polyporus, Ceriporiopsis subvermispora, Ganoderma tsunodae and Trichoderma.

In some embodiments, laccases include Cerrena laccase A1, B1 and D2 from CBS115.075 strain, Cerrena laccase A2, B2, C, D1, and E from CBS154.29 strain, Cerrena laccase B3 enzyme from ATCC20013 strain (see e.g., US Publication No. 2008/0196173, incorporated herein by reference in its entirety). Further optimized versions of these laccases also find use in the present invention.

In another embodiments, laccases include the mature protein of Cerrena laccase D expressed in Trichoderma; the amino acid sequence of which is shown as follows (SEQ ID NO: 45).

AIGPVADLHIVNKDLAPDGVQRPTVLAGGTFPGTLITGQKGDNFQLNVID
DLTDDRMLTPTSIHWHGFFQKGTAWADGPAFVTQCPIIADNSFLYDFDVP
DQAGTFWYHSHLSTQYCDGLRGAFVVYDPNDPHKDLYDVDDGGTVITLAD
WYHVLAQTVVGAATPDSTLINGLGRSQTGPADAELAVISVEHNKRYRFRL
VSISCDPNFTFSVDGHNMTVIEVDGVNTRPLTVDSIQIFAGQRYSFVLNA
NQPEDNYWIRAMPNIGRNTTTLDGKNAAILRYKNASVEEPKTVGGPAQSP
LNEADLRPLVPAPVPGNAVPGGADINHRLNLTFSNGLFSINNASFTNPSV
PALLQILSGAQNAQDLLPTGSYIGLELGKVVELVIPPLAVGGPHPFHLHG
HNFWVVRSAGSDEYNFDDAILRDVVSIGAGTDEVTIRFVTDNPGPWFLHC
HIDWHLEAGLAIVFAEGINQTAAANPTPQAWDELCPKYNGLSASQKVKPK
KGTAI

### Host Cells

The present invention provides host cells transformed with DNA constructs and vector as described herein. In some embodiments, the present invention provides for host cells transformed with DNA constructs encoding a desired protein and operably linked to the NSP24 or CBHI signal peptide as described herein. In some embodiments, the invention provides DNA constructs that encode at least one desired protein such as protease, laccase, alpha amylase, glucoamylase, xylanase, and cellulose, wherein the constructs are introduced into a host cell. In some embodiments, the present invention provides for the expression of protein genes and/or overexpression of protein genes under control of gene promoters functional in bacterial and/or fungal host cells.

It is intended that any suitable host cell are useful with the present invention. It is not intended that the present invention be limited to any particular host cell. In some embodiments, the host cell is a cell in which the signal peptide has activity in secreting the protein of interest. For example, host cells for which a T. reesei signal peptide find use include, but are not limited to, fungal and bacterial cells. Host cells include filamentous fungal cells, including but not limited to Trichoderma spp. (e.g., T. viride and T. reesei, the asexual morph of Hypocrea jecorina, previously classified as T longibrachiatum), Penicillium spp., Humicola spp. (e.g., H. insolens and H. grisea), Aspergillus spp. (e.g., A. niger, A. nidulans, A. orzyae, and A. awamori), Fusarium spp. (e.g., F. graminum), Neurospora spp., Hypocrea spp. and Mucor spp. Alternative host cells include, but are not limited to Bacillus spp (e.g., B. subtilis, B. licheniformis, B. lentus, B. stearothremophilus and B. brevis) and Streptomyces spp. (e.g., S. coelicolor and S. lividans).

Many methods are known in the art for identifying whether a protein is secreted in a host cell or remains in the cytoplasm. It is intended that any suitable method will find use in identifying host cells in which the signal sequence is active.

### Protein Expression

Desired proteins of the present invention are produced by culturing cells transformed with a vector such as an expression vector containing genes whose secretion is enhanced by the NSP24 or CBH1 signal peptide sequence, foldases, chaperonins, and/or chaperones. The present invention is particularly useful for enhancing the intracellular and/or extracellular production of proteins. As those of skill in the art know, optimal conditions for the production of the proteins will vary with the choice of the host cell and protein to be expressed. Such conditions are easily determined by those of skill in the art.

In some embodiments, the protein of interest is isolated or recovered and purified after expression. Various methods for protein isolation and purification are known to those of skill in the art. Any suitable method finds use in the present invention. For example, standard purification methods that find use in the present invention include, but are not limited to electrophoretic, molecular, immunological and chromatographic techniques, including ion exchange, hydrophobic, affinity, and reverse-phase HPLC chromatography, and chromatofocusing. For example, in some embodiments, the protein of interest is purified using a standard antibody column comprising antibodies directed against the protein of interest. Ultrafiltration and diafiltration techniques, in conjunction with protein concentration, also find use in some embodiments. As known to those of skill in the art, the degree of purification necessary varies depending on the use of the protein of interest. Indeed, in some embodiments, no purification is necessary.

In some embodiments, proteins of interest produced by transformed host cells, as provided by the present invention, are recovered from the culture medium by conventional procedures known to those of skill in the art. These methods include, but are not limited to separating the host cells from the medium by centrifugation or filtration. In some embodiments, the cells are disrupted and the supernatant is removed from the cellular fraction and debris. In some embodiments, the proteinaecous components of the supernatant or filtrate are precipitated by means of a salt (e.g., ammonium sulfate) after clarification. The precipitated proteins are then solubilized and in some embodiments, are purified by any suitable method, including chromatographic procedures (e.g., ion exchange chromatography, gel filtration chromatography, affinity chromatography, and other art-recognized procedures).

In some further embodiments, antibodies directed against the peptides and proteins produced using the present invention are generated by immunizing an animal (e.g., a rabbit or mouse), and recovering anti-protein and/or NSP24 signal peptide antibodies using any suitable method known in the art. In some additional embodiments, monoclonal antibodies are produced using any suitable method known in the art.

In some embodiments, assays known to those of skill in the art find use in the present invention, including, but not limited to those described in WO 99/34011 and U.S. Pat. No. 6,605,458, both of which are incorporated by reference herein in their entirety.

### Fusions

In some embodiments, the desired protein is produced as a fusion protein. In some further embodiments, the desired protein is fused to a protein that is efficiently secreted by a filamentous fungus, and fused to an enzyme catalytic domain from the same phylum, genus, and/or species as the host cell used for expression of the fusion protein. In some embodiments, the desired protein is fused to a CBHI polypeptide, or portion thereof. In some additional embodiments, the desired protein is fused to a CBHI polypeptide, or portion thereof, that is altered to minimize or eliminate catalytic activity. In some still further embodiments, the desired protein is fused to a Trichoderma glucoamylase polypeptide, or portion thereof. In some additional embodiments, the desired protein is fused to a Trichoderma glucoamylase, or portion thereof, that is altered to minimize or eliminate catalytic activity. In some further embodiments, the desired protein is fused to a polypeptide to enhance secretion, facilitate subsequent purification and/or enhance stability.

In general, the first, second, and/or third polynucleotide in the expression host of the present invention is either genetically inserted or integrated into the genomic makeup of the expression host (e.g., it is integrated into the chromosome of the expression host). However, in some embodiments, it is extrachromosomal (e.g., it exists as a replicating vector within the expression host). In some further embodiments, the extrachromosomal polynucleotide is expressed under suitable selection conditions for a selection marker that is present on the vector).

### Secretion Level Assays

As described herein, the secretion level of a desired polypeptide in the expression host is determined using any suitable method. For example, in some embodiments, the secretion level is based on various factors (e.g., growth conditions of the host), etc. However, in some embodiments, the secretion level of the desired polypeptide expressed in the host is higher than the secretion level of the desired polypeptide expressed without the presence of a secretion enhancing protein. In some embodiments, the secretion level of a desired polypeptide (e.g., laccase from Cerrena unicolor in an expression host such as T. reesei) is at least about 1 mg/liter, about 2 mg/liter, about 3 mg/liter, about 4 mg/liter, or about 5 mg/liter when the host is grown in batch fermentation mode in a shake flask, or at least about 50 mg/liter, about 100 mg/liter, about 150 mg/liter, about 200 mg/liter, about 250 mg/liter, about 500 mg/liter, about 1000 mg/liter, about 2000 mg/liter, about 5000 mg/liter, about 10,000 mg/liter or about 20,000 mg/liter when the host is grown in a fermenter environment with controlled pH, feed-rate, etc. (e.g., fed-batch fermentation).

For example, in order to evaluate the expression and/or secretion of a secretable polypeptide, assays are carried out at the protein level, the RNA level, and/or through the use of functional bioassays suitable for the secretable polypeptide activity and/or production. Exemplary assays employed to analyze the expression and/or secretion of secretable polypeptide include but are not limited to, Northern blotting, dot blotting (DNA or RNA analysis), RT-PCR (reverse transcriptase polymerase chain reaction), or in situ hybridization, using an appropriately labeled probe (based on the nucleic acid coding sequence), conventional Southern blotting and autoradiography.

In some embodiments, the production, expression and/or secretion of a secretable polypeptide is directly measured in a sample. In some embodiments, the measurements are made using assays for enzyme activity, expression and/or production. In some embodiments, protein expression is evaluated by immunological methods (e.g., immunohistochemical staining of cells and/or tissue sections, or immunoassays of tissue culture medium by Western blotting or ELISA methods). Such immunoassays find use in qualitatively and/or quantitatively evaluating the expression of secretable polypeptide. These methods are known to those of skill in the art. Indeed, there are numerous commercially available kits and reagents for use in such methods.

In some embodiments, the present invention also provides extracts (e.g., solids or supernatants) obtained from the culture medium used to grow the expression host. In some embodiments, the supernatant does not contain substantial amount of the expression host, while in some alternative embodiments, the supernatant does not contain any amount of the expression host.

### Cell Culture

As known in the art, the host cells and transformed cells of the present invention can be cultured in conventional nutrient media. However, in some embodiments, the culture media for transformed host cells is modified as appropriate, for activating promoters and selecting transformants. The specific culture conditions, such as temperature, pH and the like, are typically those that are used for the host cell selected for expression, and will be apparent to those skilled in the art. Culture media and conditions for host cells are known to those of skill in the art. It is noted that in culture, stable transformants of fungal host cells, such as Trichoderma cells are generally distinguishable from unstable transformants by their faster growth rate or the formation of circular colonies with a smooth, rather than ragged outline on solid culture medium.

### Compositions

In some embodiments, the present invention provides compositions and methods for expressing desired proteins using the NSP24 or CBH1 signal sequence, constructs and vectors. In some embodiments, the present invention provides compositions that include enzymes, including, but not limited to laccases, glucoamylases, alpha amylases, granular starch hydrolyzing enzymes, cellulases, lipases, phospholipases, xylanases, cutinases, hemicellulases, oxidases, peroxidases, proteases, phytases, keratinases, pullulanases, glucoamylases, pectinases, oxidoreductases, reductases, perhydrolases, phenol oxidases, lipoxygenases, ligninases, tannanases, pullulanases, pentosanases, beta-glucanases, arabinosidases, hyaluronidases, chondrointinases, mannanases, esterases, acyl transferases, and combinations thereof.

### Applications

The desired proteins produced by the present invention find use in any applications appropriate for that protein. Examples of applications for proteins such as enzymes include, but are not limited to animal feeds for improvement of feed intake and feed efficiency (e.g., proteases), dietary protein hydrolysates (e.g., for individuals with impaired digestive systems), leather treatment, treatment of protein fibers (e.g., wool and silk), cleaning, protein processing (e.g., to remove bitter peptides, enhance the flavor of food, and/or to produce cheese and/or cocoa), personal care products (e.g., hair compositions), sweeteners (e.g., production of high maltose or high fructose syrups), fermentation and bioethanol (e.g., alpha amylases and glucoamylases used to treat grains for fermentation to produce bioethanol). Examples of applications for laccases include, but are not limited to bleaching of pulp and paper, textile bleaching, treatment of waste water, de-inking of waste paper, polymerization of aromatic compounds or proteins, radical-mediated polymerization and cross-linking reactions (e.g., paints, coatings, biomaterials), the activation of dyes, and to couple organic compounds. The laccases also find use in cleaning composition, including but not limited to laundry and other detergents.

### EXAMPLES

The following examples are offered to illustrate, but not to limit the claimed invention. It will be apparent to those skilled in the art that many modifications, both to materials and methods, may be practiced without departing from the scope of the invention.

In the experimental disclosure which follows, the following abbreviations apply: M (Molar); μM (micromolar); N (Normal); mol (moles); mmol (millimoles); μmol (micromoles); nmol (nanomoles); g (grams); mg (milligrams); kg (kilograms); μg and ug (micrograms); L (liters); ml (milliliters); μl and ul (microliters); cm (centimeters); mm (millimeters); μm (micrometers); nm (nanometers); ° C. (degrees Centigrade); h and hr (hours); min (minutes); sec (seconds); msec (milliseconds); V (voltage); xg (times gravity); ° F. (degrees Fahrenheit); amdS (acetamidase, a selective marker obtained from A. nidulans); lccD (laccase); BioRad (BioRad Laboratories, Hercules, Calif.); Difco (Difco Laboratories, Detroit, Mich.); Calbiochem (Calbiochem brand owned by EMD Chemicals Inc., San Diego, Calif.); Sigma (Sigma Chemical Co., St. Louis, Mo.); Spectronic (Spectronic Devices, Ltd., Bedfordshire, UK); Advanced Kinetics (Advanced Kinetics and Technology Solutions, Switzerland).

Most of the expression vectors in the examples were produced based on the pSL1180 plasmid backbone, the sequence of which is provided in the GENBANK® database, under the identifier U13865. The markers such as the amdS marker, chaperones or foldases, laccase (lccD), the signal sequences, TrGA fusions and terminators were added using the polylinker and/or PCR methods as known in the art.

The sites on the plasmids are identified as follows: cbh1—cellobiohydrolase; Tcbh1—the terminator from cbh1; TrGA—Trichoderma glucoamylase; lccD—laccase D; amdS marker selectable marker for autotrophism; pSL1180—the plasmid backbone; laccase D opt—an optimized version of the laccase D gene that is constructed with codon usage optimized for expression in the host (Trichoderma); Pcpc-1—a promoter from the cross pathway control-1 gene from Neurospora crassa; bla—β-lactamase gene (i.e., a selective marker from E. coli); and HphR—the hygromycin-resistance gene (a selective marker from E. coli).

To construct the expression plasmids, primers were designed and used in the Herculase PCR reaction (Stratagene) containing the DNA template.

### Example 1

### Construction of Expression Vector pTrex4-laccaseD opt

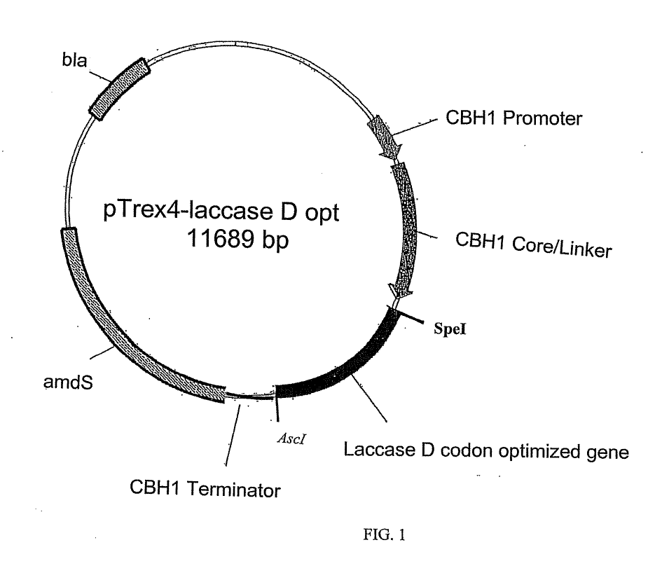

This Example describes the steps involved in the construction of the expression vector pTrex4-laccaseD opt. The plasmid was produced to express the codon optimized laccase D gene from C. unicolor using the CBH1 promoter and CBH1 signal sequence. This expression vector contained the laccase D codon optimized gene fused to the CBH1 (cellobiohydrolase) core/linker and expressed from the CBH1 promoter. FIG. 1 provides a schematic of the Trichoderma expression plasmid. The sequence of the pTrex4-laccaseD opt plasmid is shown as SEQ ID NO: 1. The following segments of DNA were assembled in the construction of pTrex4-laccase D opt (See, FIG. 1). A fragment of T reesei genomic DNA representing the CBH1 promoter and the CBH1 signal sequence and CBH1 core/linker was inserted into the plasmid pSL1180 vector. A codon optimized copy of the C. unicolor laccase D (laccase D opt) gene was inserted, such that it was operably linked to the CBH1 at its linker region. A CBH1 terminator from T. reesei was operably linked to the laccase D gene. The amdS gene was added as a selectable autotropic marker. The bla gene (encoding beta-lactamase, a selective marker obtained from E. coli) is present in the pSL1180 vector.

### Example 2

### Construction of Expression Vector pTrex2g-Bip1

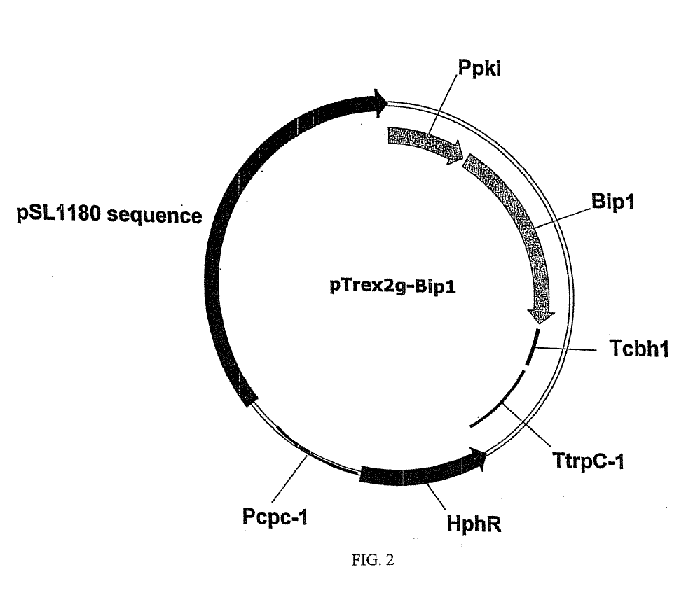

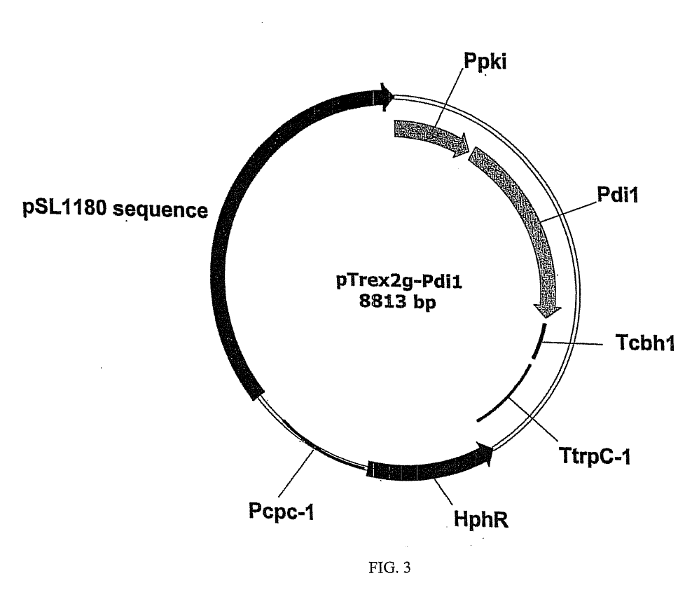

The pTrex2g/Bip1 plasmid was produced to express the bip1 chaperone from T. reesei. FIG. 2 provides the schematic of the Trichoderma expression plasmid pTrex2g-Bip1; The sequence of the plasmid is provided as SEQ ID NO: 2. The following segments of DNA were assembled in the construction of pTrex2g-Bip1. A 2267 bp fragment of T. reesei bip1 was inserted into the plasmid pSL1180 vector operably linked to the Ppki promoter (pyruvate kinase from T. reesei), The Trichoderma cbh1 terminator was operably linked to the bip1 gene. The HphR selectable marker from E. coli was included for selection and was operably linked to the Pcpc-1 promoter (cross pathway control-1 gene from Neurospora crassa) and the trpC terminator (tryptophan synthesis gene C from A. nidulans).

### Example 3

### Construction of Expression Vector pTrex2g-Pdi1

The pTrex2g-Pdi 1 plasmid was produced to express the chaperone pdi1 in the same way as the pTrex2g-Bip1 (See, Example 2), except that the T. reesei pdi1 chaperone gene (2465 bp) was inserted in place of the bip1 chaperone gene. FIG. 3 provides the schematic of the Trichoderma expression plasmid pTrex2g-Pdi 1; the sequence of the plasmid is provided as SEQ ID NO: 3.

### Example 4

### Construction of Expression Vector pTrex2g-Ero1

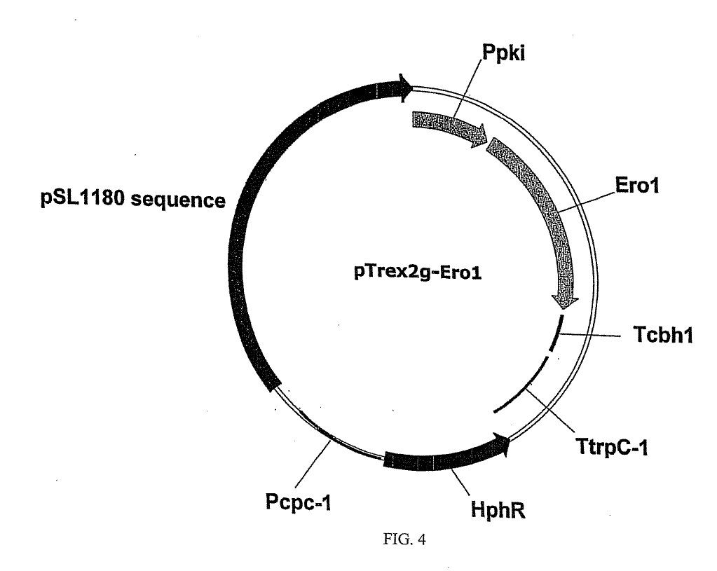

The pTrex2g-Ero1 plasmid was produced to express the chaperone ero1 in the same way as the pTrex2g-Bip1 (See, Example 2), except that the T. reesei ero1 chaperone gene (2465 bp) was inserted in place of the bip1 chaperone gene. FIG. 4 provides the schematic of the ero1 in the Trichoderma expression plasmid pTrex2g-Ero1. The sequence of ero1 is provided as SEQ ID NO: 4.

### Example 5

### Construction of Expression Vector pTrGA-laccaseD opt

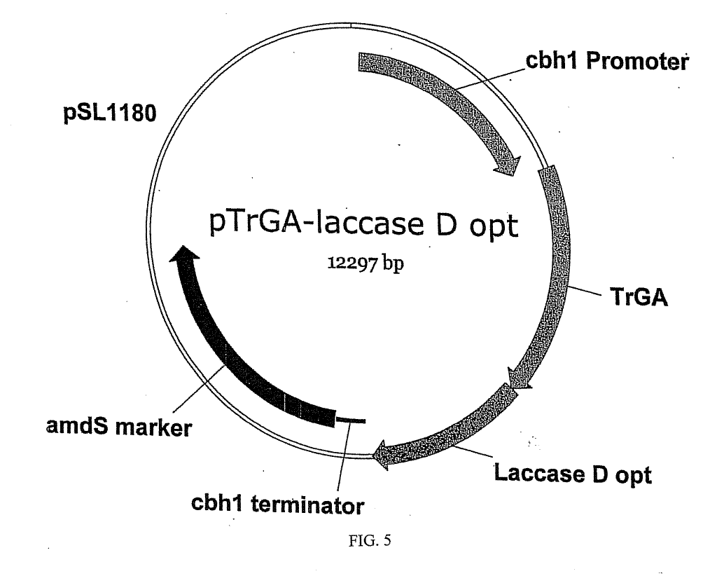

The pTrGA-laccaseD opt plasmid was produced similarly to that in Example 1, except that pTrGA-laccase D opt expresses a fusion of the full-length glucoamylase from T. reesei and C. unicolor laccase D with optimized codons. FIG. 5 provides the schematic of the Trichoderma expression plasmid pTrGA-laccaseD opt; the polynucleotide sequence is shown as SEQ ID NO:5.

### Example 6

### Construction of Expression Vector pKB408

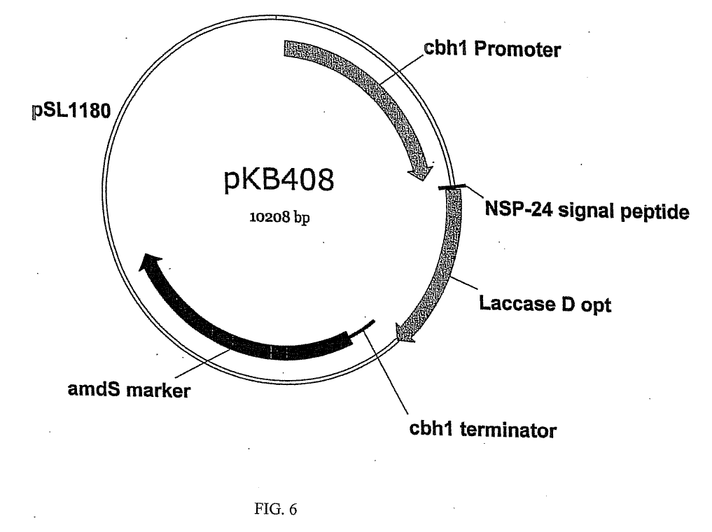

The pKB408 plasmid was produced to express C. unicolor laccase D opt operably fused to the T. reesei NSP-24 signal peptide. The plasmid was constructed similarly to that shown in FIG. 1 except that the laccase D constructs were operably linked to the NSP-24 signal peptide, which was inserted in place of the laccase D opt linked to the CBH1 signal sequence, catalytic domain and linker. FIG. 6 provides the schematic of the Trichoderma expression plasmid pKB408; the polynucleotide sequence is shown as SEQ ID NO: 6.

### Example 7

### Construction of Expression Vector pKB410

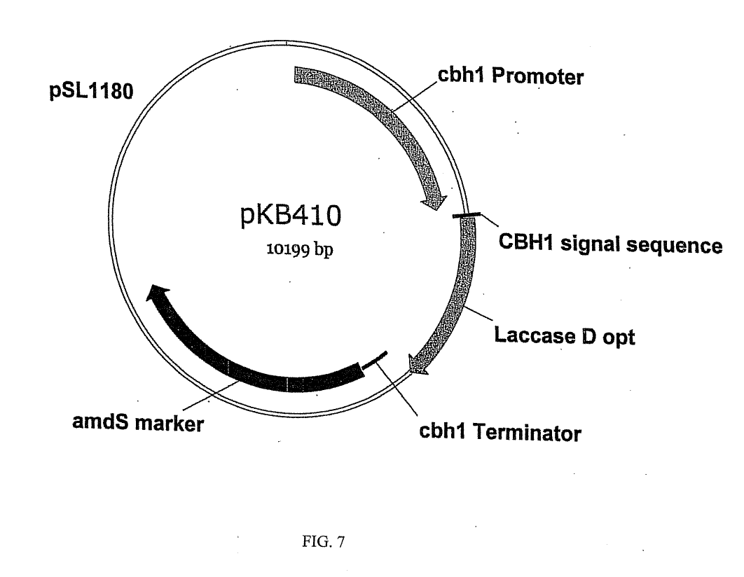

The pKB410 plasmid was produced as described in Example 6, except the T. reesei CHB1 signal sequence was used instead of the NSP-24 signal sequence. FIG. 7 provides the schematic of the Trichoderma expression plasmid pKB410; the polynucleotide sequence is shown as SEQ ID NO: 7.

### Example 8

### Transformation of T. reesei and Analysis of Expression

In this example, the stable recombinant T. reesei strain derived from RL-P37 (See, Sheir-Neiss and Montenecourt, Appl. Microbiol. Biotechnol., 20:46-53 (1984)) and deleted for the cbh1, cbh2, egl1, and egl2 genes described by Bower et al (See, Bower et al., Carbohydrases From Trichoderma reesei and Other Micro-organisms, Royal Society of Chemistry, Cambridge, pp. 327-334 (1998)) was used for transforming the plasmids from Examples 1-14 alone or in various combinations. Biolistic and electroporation methods were used to transform the plasmids, as described below.

### Biolistic Transformation

The expression plasmid was confirmed by DNA sequencing and transformed biolistically into a Trichoderma strain. Transformation of the Trichoderma strain by the biolistic transformation method was accomplished using a Biolistic® PDS-1000/The Particle Delivery System (Bio-Rad) following the manufacturer's instructions (See, WO 05/001036 and US Pat. Appl. Publ. No. 2006/0003408). Transformants were selected and transferred onto minimal media with acetamide (MMA) plates and grown for 4 days at 28-30° C. A small plug of a single colony including spores and mycelium was transferred into 30 mls of NREL lactose defined broth (pH 6.2) containing 1 mM copper. The cultures were grown for 5 days at 28° C. Culture broths were centrifuged and supernatants were analyzed using the ABTS assay as described below for laccase activity.

### Electroporation

Electroporation was performed as described in U.S. Patent application No. 60/931,072, herein incorporated by reference in its entirety. A T. reesei strain was grown and sporulated on Potato Dextrose Agar plates (Difco) for about 10-20 days. The spores were washed from the surface of the plates with water and purified by filtration through Miracloth (Calbiochem). The spores were collected by centrifugation (3000×g, 12 min), washed once with ice-cold water and once with ice-cold 1.1M sorbitol. The spore pellet was re-suspended in a small volume of cold 1.1 M sorbitol, mixed with about 8 μg of gel-purified DNA fragment isolated from plasmid DNA (pKB408 and pKB410, FIGS. 6 and 7) per 100 μl of spore suspension. The mixture (100 μl) was placed into an electroporation cuvette (1 mm gap) and subjected to an electric pulse using the following electroporation parameters: voltage 6000-20000 V/cm, capacitance=25 μF, resistance=50Ω. After electroporation, the spores were diluted about 100-fold into 5:1 mixture of 1.1 M sorbitol and YEPD (1% yeast extract, 2% Bacto-peptone, 2% glucose, pH 5.5), placed in shake flasks and incubated for 16-18 hours in an orbital shaker (28° C. and 200 rpm). The spores were once again collected by centrifugation, re-suspended in about 10-fold of pellet volume of 1.1 M sorbitol and plated onto two 15 cm Petri plates containing amdS modified medium (acetamide 0.6 g/l, cesium chloride 1.68 g/l, glucose 20 g/l, potassium dihydrogen phosphate 15 g/l, magnesium sulfate heptahydrate 0.6 g/l, calcium chloride dihydrate 0.6 g/l, iron (II) sulfate 5 mg/l, zinc sulfate 1.4 mg/l, cobalt (II) chloride 1 mg/l, manganese (II) sulfate 1.6 mg/l, agar 20 g/l and pH 4.25). Transformants appeared at about 1 week of incubation at 28-30° C.

The ABTS assay was performed as follows: An ABTS stock solution was prepared containing 4.5 mM ABTS in water (ABTS; Sigma Cat# A-1888). Buffer was prepared containing 0.1 M sodium acetate pH 5.0. Then, 1.5 ml of buffer and 0.2 ml of ABTS stock solution were added to cuvettes (10×4×45 mm, No./REF67.742) and mixed well. One extra cuvette was prepared as a blank. Then, 50 ul of each enzyme sample to be tested (using various dilutions) were added to the mixtures.

The ABTS activity was measured in a Genesys2 machine (Spectronic) using an ABTS kinetic assay program set up: (Advanced Kinetics) as follows: wave length 420 nm, interval time (Sec) 2.0, total run time (sec) 14.0, factor 1.000, low limit—000000.00, high limit 999999.00, and the reaction order was first.

The procedure involved adding 1.5 mL of NaOAc (120 mM NaOAc Buffer pH 5.0), then add 0.2 mL of 4.5 mM ABTS to the cuvette, then to blank the cuvette, adding 0.05 mL of the enzyme sample to the cuvette, mixing quickly and well and, finally, measuring the change of absorption at 420 nm, every 2 seconds for 14 seconds. One ABTS unit is defined as change of A420 per minute (given no dilution to the sample). Calculation of ABTS U/mL: (change in Δ420/min*dilution factor).

### Example 9

### Analysis of Laccase/Glucoamylase Fusion Gene Expression in T. reesei Transformants

The culture medium of the transformants obtained and cultivated as described in Example 8 was separated from mycelium by centrifugation (16000×g, 10 min) and ABTS activity from the supernatants were analyzed. The results are shown in FIG. 10. Table 3 provides the strains described in FIG. 10. FIG. 10 illustrates the improvement of laccase production by fusion of the gene encoding C. unicolor laccase to the full-length Trichoderma glucoamylase. The results showed that expression of laccase improved 24-29% when fused to the Trichoderma glucoamylase, than fused to CBH1.

TABLE 3
Strains Used in FIG. 10

| Strain Identification Number | Strain Type |
|---|---|
| #8-2 | CBH1 laccase fusion |
| 1066-9 | TrGA laccase fusion |
| 1066-13 | TrGA laccase fusion |
| 1066-15 | TrGA laccase fusion |

### Example 10

### Analysis of Laccase Production Using NSP24 and CBH1 Signal Sequences

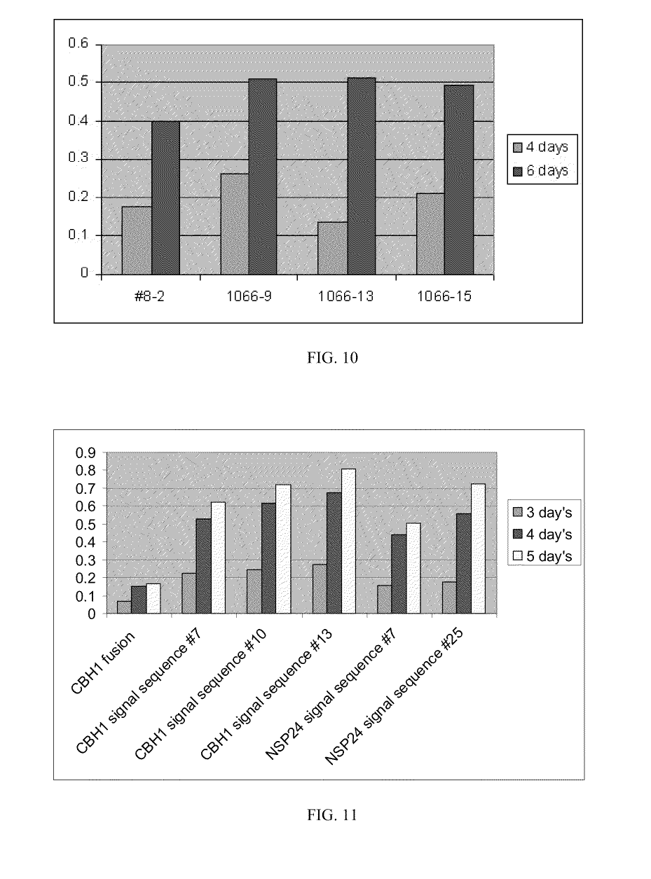

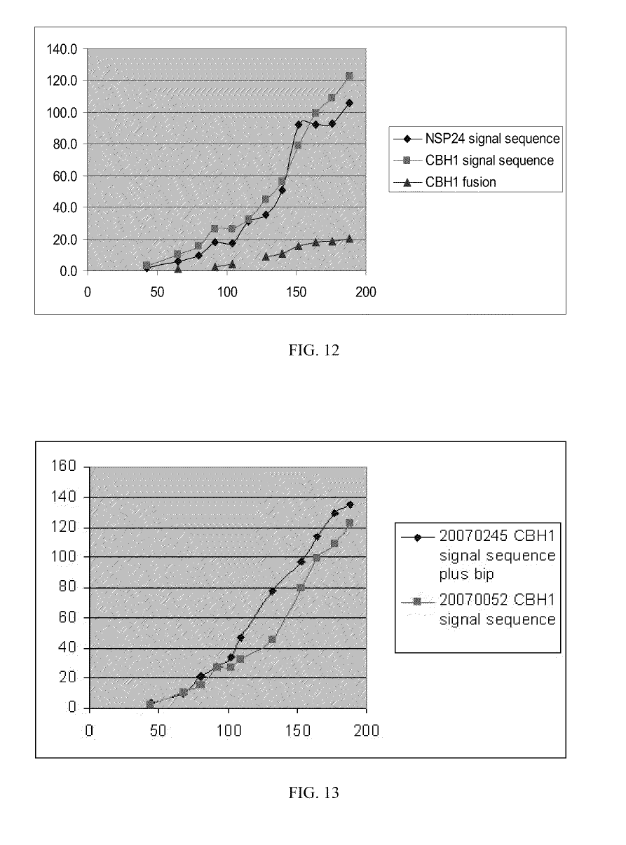

When the T. reesei CBH1 signal sequence was operably linked to the laccase gene, expression was improved 4-5 folds over initial CBH1 fusion strain #8-2 alone in shake flasks and 5-6 folds in a 14 liter fermentor as shown by the results provided in FIGS. 11 (shake flasks) and 12 (fermentor). When the T. reesei NSP-24 signal sequence was used, the expression improved 3-4 folds in shake flasks and 4-5 folds in a 14 liter fermentor. Three clones were analyzed in the shake flasks for the CBH1 signal sequence (#7, #10, and #13) and two clones were analyzed for the NSP24 signal sequence (#7 and #25) and the expression was analyzed at 3 days (first bar), 4 days (second bar) and 5 days (third bar). A single clone of each was analyzed in the 14 liter fermenters, as shown by the results in FIG. 12. In this Figure, the diamond indicates the NSP24 signal sequence operably linked to the laccase D, the square indicates the CBH1 signal sequence operably linked to the laccase D and the triangle indicates the CBH1 fusion alone.

### Example 11

### Analysis of Laccase Production Using CBH1 Signal Sequence and Co-Expression of bip1 in a Fermenter

The CBH1 signal sequence plasmid (operably linked to laccase) was co-transformed with the T. reesei Bip1 plasmid and expression analyzed. The results are shown in FIG. 13. In FIG. 13, diamonds indicate the data obtained for the CHB1 signal sequence (operably linked to laccase) plus BIP1, while the squares indicate the data obtained for the CBH1 signal sequence (operably linked to laccase) alone. FIG. 13 illustrates the improvement of laccase production provided by the CBH1 signal sequence plus BIP1 chaperone expression, which increased expression significantly, by more than 15% in fermentors.

### Example 12

### Analysis of Laccase Production Using CBH1 Signal Sequence and Co-Expression of bip1 in a Shake Flask

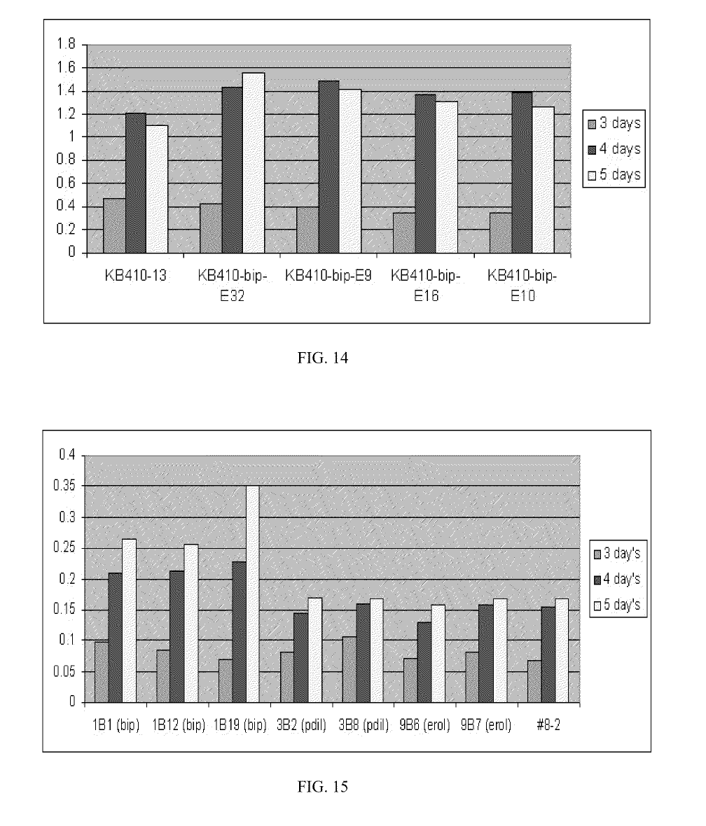

The CBH1 signal sequence plasmid (operably linked to laccase) was co-transformed with the T. reesei bip1 plasmid, grown in and laccase expression analyzed using the ABTS assay. The results are presented in FIG. 14. Five different clones were analyzed for 3 days (first bar) 4 days (second bar) and 5 days (third bar). KB410-13 was a control having CBH1 signal sequence plasmid alone. The other 4 clones were KB410-13 with one of the bip1 co-transformants: E32, E9, E16, and E10. FIG. 14 illustrates the improvement of laccase production by co-expression of chaperones with C. unicolor in shake flasks. The co-expression with bip1 increased expression significantly (from 14-41%) in shake flasks.

### Example 13

### Analysis of Laccase Production Using CBH1-laccase D Fusion and Co-Expression of a Variety of Chaperones

The expression plasmid having a CBH1 signal sequence, catalytic domain and linker operably linked to laccase was co-transformed with a variety of T. reesei chaperone plasmids (BIP1, PDI1, and ERO1). The resultant transformed cell was grown in culture and laccase expression analyzed. FIG. 15 illustrates the improvement of laccase production by fusion of the gene encoding C. unicolor laccase to the CBH1 signal sequence, catalytic domain and linker and co-expression with bip1, pdi1 and ero1 chaperones.

All strains had CBH1 signal sequence, catalytic domain and linker linked to laccase D. Strains 1B1, 1B12 and 1B19 had bip1 expression cassette; they were three independent transformants, with difference in the bip1 plasmid copy numbers and location of integration. Strains 3B2 and 3B8 had pdi1 expression cassette; they are two independent transformants, with difference in the pdi1 plasmid copy numbers and location of integration. Strains 9B6 and 9B7 had ero1 expression cassette; they are two independent transformants, with difference in the ero1 plasmid copy numbers and location of integration may be different. #8-2 is the control strain which has no chaperone expression cassette.

The results of FIG. 15 indicate that the highest increase in expression was obtained with the co-expression with the bip1 chaperone.

### Example 14

### Analysis of Laccase Production Using CBH1 Signal Sequence and Co-Expression of a Variety of Chaperones

The CBH1 signal sequence plasmid (i.e., operably linked to laccase) was co-transformed with a variety of T. reesei chaperone plasmids (bip1, lhs1, pdi1, ppi1, ppi2, tig1, prp1, and ero1), either alone or in combination. The cultures were grown in shake flasks as known in the art and laccase expression analyzed using the ABTS assay. The clones were analyzed in triplicate. The data provided in Table 4 show that adding more than one chaperone did not increase expression of laccase above that of bip1 alone. The data in Table 4 show three independent spore-purified samples (or clones) from the same strain.

TABLE 4
Expression of Laccase in the Presence of Chaperones
Co-transformation of KB413-32A with Different Chaperones
Each Strain has 3 repeats: -A, -B, -C

| | | | 4 days | 6 |
|---|---|---|---|---|
| | Samples | Chaperones | SF broth | days |
| 1 | KB413-32A-A | bip1 only | 4.52 | 6.32 |
| 2 | KB413-32A-B | bip1 only | 4.26 | 6.35 |
| 3 | KB413-32A-C | bip1 only | 4.28 | 6.13 |
| 4 | KB414-1-A | bip1, ero1 | 3.88 | 5.89 |
| 5 | KB414-1-B | bip1, ero1 | 3.78 | 5.93 |
| 6 | KB414-1-C | bip1, ero1 | 3.76 | 5.59 |
| 7 | KB415-2-A | bip1, lhs1, white | 3.8 | 5.93 |
| 8 | KB415-2-B | bip1, lhs1, white | 3.72 | 5.92 |
| 9 | KB415-2-C | bip1, lhs1, white | 3.78 | 6.06 |
| 10 | KB415-3-A | bip1, lhs1, gray | 4.38 | 6.32 |
| 11 | KB415-3-B | bip1, lhs1, gray | 4.3 | 6.66 |
| 12 | KB415-3-C | bip1, lhs1, gray | 3.98 | 6.15 |
| 13 | KB416-3-A | bip1, pdi1 | 4.18 | 6.58 |
| 14 | KB416-3-B | bip1, pdi1 | 5.26 | 7.12 |
| 15 | KB416-3-C | bip1, pdi1 | 4.22 | 6.06 |
| 16 | KB417-3-A | bip1, ppi1 | 4.32 | 6.23 |
| 17 | KB417-3-B | bip1, ppi1 | 3.96 | 6.32 |
| 18 | KB417-3-C | bip1, ppi1 | 4.18 | 6.88 |
| 19 | KB418-2-A | bip1, ppi2 | 4.24 | 6.59 |
| 20 | KB418-2-B | bip1, ppi2 | 3.96 | 5.69 |
| 21 | KB418-2-C | bip1, ppi2 | 4.04 | 5.92 |
| 22 | KB419-1-A | bip1, tigA | 4.66 | 5.98 |
| 23 | KB419-1-B | bip1, tigA | 5.26 | 7.25 |
| 24 | KB419-1-C | bip1, tigA | 4.18 | 6.05 |
| 25 | KB413-prp2-A | bip1, prpA | 3.96 | 5.63 |
| 26 | KB413-prp2-B | bip1, prpA | 3.9 | 5.59 |
| 27 | KB413-prp2-C | bip1, prpA | 3.92 | 5.86 |
| 28 | KB414-1-A | bip1, ero1 | 4.2 | 6.01 |
| 29 | KB414-1-B | bip1, ero1 | 3.88 | 5.69 |
| 30 | KB414-1-C | bip1, ero1 | 3.92 | 5.88 |

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A method for producing a desired protein, comprising the steps of:
(a) introducing into a host cell a first nucleic acid sequence comprising a signal sequence operably linked to a desired protein sequence;
(b) expressing the first nucleic acid sequence;
(c) co-expressing a second nucleic acid sequence encoding a chaperone or foldase selected from the group consisting of bip1, ero1, pdi1, tig1, prp1, ppi1, ppi2, prp3, prp4, calnexin, and lhs1; and
(d) collecting the desired protein secreted from the host cell.

2. The method according to claim 1, wherein the first nucleic acid sequence further comprises an enzyme sequence between the signal sequence and the desired protein sequence.

3. The method according to claim 2, wherein the enzyme sequence is obtained from a glucoamylase or from a CBH1 enzyme.

4. The method according to claim 2, wherein the enzyme sequence comprises a full-length enzyme sequence.

5. The method according to claim 2, wherein the enzyme sequence comprises a catalytic core domain sequence.

6. The method according to claim 5, wherein the first nucleic acid sequence further comprises a linker sequence between the catalytic core domain sequence and the desired protein sequence.

7. The method according to claim 1, wherein the desired protein is a laccase.

8. The method according to claim 7, wherein said laccase is derived from a filamentous fungus or yeast.

9. The method according to claim 8, wherein said laccase is derived from Aspergillus, Neurospora, Podospora, Botrytis, Collybia, Cerrena, Stachybotrys, Panus, Thieilava, Fomes, Lentinus, Pleurotus, Trametes, Rhizoctonia, Coprinus, Psatyrella, Myceliophthora, Schytalidium, Phlebia, Coriolus, Spongipellis, Polyporus, Ceriporiopsis subvermispora, Ganoderma tsunodae, or Trichoderma.

10. The method according to claim 9, wherein said laccase is derived from Cerrena laccase A1, A2, B1, B2, B3, C, D1, D2, or E.

11. The method according to claim 9, wherein said laccase is derived from the mature protein of Cerrena laccase D.

12. The method according to claim 1, wherein the signal sequence encodes Cellobiohydrolase I signal peptide or NSP24 signal peptide.

13. The method according to claim 1, wherein the host is a filamentous fungus.

14. The method according to claim 13, wherein the host is ascomycetes.

15. The method according to claim 14, wherein the host is Trichoderma.

16. The method according to claim 1, wherein the first nucleic acid sequence further comprises a promoter upstream to a signal sequence.

17. The method according to claim 16, wherein the promoter is native to the host cell and is not naturally associated with the desired protein sequence.

18. The method according to claim 1, wherein the chaperon is BIP 1.

19. The method according to claim 1, wherein the second nucleic acid sequence is operably linked to a promoter.

20. The method according to claim 19, wherein the promoter is native to the host cell and is not naturally associated with the second nucleic acid sequence.

21. The method according to claim 2, wherein the desired protein is a laccase and the laccase is produced as a fusion protein with the enzyme.


SEQUENCE LISTING (45 sequences)

SEQ ID NO: 1 - DNA, 11689 bp, Trichoderma
aagcgcctgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgacgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcatcatgtatcggaagttggccgtcatctcggccttcttggccacagctcgtgctcagtcggcctgcactctccaatcggagactcacccgcctctgacatggcagaaatgctcgtctggtggcacttgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgtatcttcttgtgggctcccagctgactggccaatttaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcaaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctactagtgtcgccgtttacaaacgcgctattggaccagttgctgatctgcacatcgttaacaaggatttggccccagacggcgtccagcgcccaactgttctggccggtggaacttttccgggcacgctgattaccggtcaaaagggcgacaacttccagctgaacgtgattgatgacctgaccgacgatcgcatgttgacccctacttcgatccattggcatggtttcttccagaagggaaccgcctgggccgacggtccggctttcgttacacagtgccctattatcgcagacaactccttcctctacgatttcgacgttcccgaccaggcgggcaccttctggtaccactcacacttgtctacacagtactgcgacggtctgcgcggtgccttcgttgtttacgaccccaacgaccctcacaaggacctttatgatgtcgatgacggtggcacagttatcacattggctgactggtatcacgtcctcgctcagaccgttgtcggagctgctacacccgactctacgctgattaacggcttgggacgcagccagactggccccgccgacgctgagctggccgttatctctgttgaacacaacaagagataccgtttcagactcgtctccatctcgtgcgatcccaacttcacttttagcgtcgacggtcacaacatgacggttatcgaggttgatggcgtgaatacccgccctctcaccgtcgattccattcaaattttcgccggccagcgatactcctttgtgctgaatgccaatcagcccgaggataactactggatccgcgctatgcctaacatcggacgaaacaccactacccttgatggcaagaatgccgctatcctgcgatacaagaacgccagcgttgaggagcccaaaaccgtcggaggacccgcgcagagcccattgaacgaggccgacctgcgacctctggtgcccgctcctgtccctggcaacgcagttcctggtggtgcggacatcaaccaccgcctgaacctgacattcagcaacggcctcttctctatcaataacgcatcatttacaaaccccagcgtccctgccttgttgcagattctttccggcgcacaaaacgctcaggatctgcttcccaccggttcttatatcggcttggagttgggcaaggtcgttgaactcgtgatccctcccttggccgttggtggcccccatccattccacttgcacggccacaacttttgggtcgtccgaagcgctggttctgacgagtataatttcgacgatgcaattttgcgcgacgtggtcagcattggcgcgggaactgacgaggttactatccgttttgtcactgataacccaggcccttggttcctccattgccacatcgactggcacctcgaagccggcctcgccattgttttcgccgaaggcatcaatcaaaccgcagccgccaacccgactccacaggcctgggacgaactctgccccaagtataacggactctccgcttcccagaaagtgaagcccaagaagggaacagccatctaaggcgcgccgcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatctaggcctattaatattccggagtatacgtagccggctaacgttaacaaccggtacctctagaactatagctagcatgcgcaaatttaaagcgctgatatcgatcgcgcgcagatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 2 - DNA, 9375 bp, Trichoderma
agactagcggccggtccccttatcccagctgttccacgttggcctgcccctcagttagcgctcaactcaatgcccctcactggcgaggcgagggcaaggatggaggggcagcatcgcctgagttggagcaaagcggccgccatgggagcagcgaaccaacggagggatgccgtgctttgtcgtggctgctgtggccaatccgggcccttggttggctcacagagcgttgctgtgagaccatgagctattattgctaggtacagtatagagagaggagagagagagagagagagagaggggaaaaaaggtgaggttgaagtgagaaaaaaaaaaaaaaaaaaaatccaaccactgacggctgccggctctgccacccccctccctccaccccagaccacctgcacactcagcgcgcagcatcacctaatcttggctcgccttcccgcagctcaggttgttttttttttctctctccctcgtcgaagccgcccttgttcccttatttatttccctctccatccttgtctgcctttggtccatctgcccctttgtctgcatctcttttgcacgcatcgccttatcgtcgtctcttttttcactcacgggagcttgacgaagacctgactcgtgagcctcacctgctgatttctctccccccctcccgaccggcttgacttttgtttctcctccagtaccttatcgcgaagccggaagaaccctctttaaccccatcaaacaagtttgtacaaaaaagcaggctatggctcgttcacggagctccctggccctcgggctgggcctgctctgctggatcacgctgctcttcgctcctctggcgtttgtcggaaaggccaatgccgcgagcgacgacgcggacaactacggcactgttatcggaattgtaagtcgactgacggcagcaaccccgccattttcttggtgttgatgctcaggcagccctgctaacacgcttctcctccgcccaggatctcggaactacctacagctgcgtcggtgtgatgcagaagggcaaggttgagattctcgtcaacgaccagggtaaccgaatcactccctcctacgtggcctttaccgacgaggagcgtctggttggcgattccgccaagaaccaggccgccgccaaccccaccaacaccgtctacgatgtcaagtcagttctaccgccctgttggcttctattgtataagtggacaattagctaactgttgtcacaggcgattgattggccgcaaattcgacgagaaggagatccaggccgacatcaagcacttcccctacaaggtcattgagaagaacggcaagcccgtcgtccaggtccaggtcaacggccagaagaagcagttcactcccgaggagatttctgccatgattcttggcaagatgaaggaggttgccgagtcgtacctgggcaagaaggttacccacgccgtcgtcaccgtccctgcctacttcaacgtgagtcttttccccgaaattcctcgaggattccaagagccatctgctaacagcccgataggacaaccagcgacaggccaccaaggacgccggtaccattgccggcttgaacgttctccgaatcgtcaacgaacccaccgctgccgctatcgcctatggtctggacaagaccgacggtgagcgccagatcattgtctacgatctcggtggtggtacctttgatgtttctctcctgtccattgacaatggcgtcttcgaggtcttggctaccgccggtgacacccaccttggtggtgaggactttgaccagcgcattatcaactacctggccaaggcctacaacaagaagaacaacgtcgacatctccaaggacctcaaggccatgggcaagctcaagcgtgaagccgaaaaggccaagcgtaccctctcttcccagatgagcactcgtatcgaaatcgaggccttcttcgagggcaacgacttctccgagactctcacccgggccaagttcgaggagctcaacatggacctcttcaagaagaccctgaagcctgtcgagcaggttctcaaggacgccaacgtcaagaagagcgaggttgacgacatcgttctggtcggcggttccacccgtatccccaaggttcagtctcttatcgaggagtactttaacggcaagaaggcttccaagggtatcaaccccgacgaggctgttgctttcggtgccgccgtccaggccggtgtcctttctggtgaggaaggtaccgatgacattgttctcatggacgtcaaccccctgactctcggtatcgagaccactggcggagtcatgaccaagctcattccccgcaacacccccatccccactcgcaagagccagatcttctcgactgctgccgataaccagcccgtcgtcctgatccaggtcttcgagggtgagcgttccatgaccaaggacaacaacctcctgggcaagttcgagcttaccggcattcctcctgccccccgcggtgtcccccagattgaggtttccttcgagttggatgccaacggtatcctcaaggtctccgctcacgacaagggcaccggcaagcaggagtccatcaccatcaccaacgacaagggccgtctcacccaggaggagattgaccgcatggttgccgaggccgagaagttcgccgaggaggacaaggctacccgtgagcgcatcgaggcccgtaacggtcttgagaactacgccttcagcctgaagaaccaggtcaatgacgaggagggcctcggcggcaagattgacgaggaggacaaggagactgtaagttgaagcgatccatcactgctttctgatgcggacatgtcacactaacacttgaccagattcttgacgccgtcaaggaggctaccgagtggctcgaggagaacggcgccgacgccactaccgaggactttgaggagcagaaggagaagctgtccaacgtcgcctaccccatcacctccaagatgtaccagggtgctggtggctccgaggacgatggcgacttccacgacgaattgtaaacccagctttcttgtacaaagtggttcgatggtttaggcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttacctgtggcgccggtgatgccggccacgatgcgtccggcgtagaggatcctctagctagaaagaaggattacctctaaacaagtgtacctgtgcattctgggtaaacgactcataggagagttgtaaaaaagtttcggccggcgtattgggtgttacggagcattcactaggcaaccatggttactattgtatacccatcttagtaggaatgattttcgaggtttatacctacgatgaatgtgtgtcctgtaggcttgagagttcaaggaagaaacagtgcaattatctttgcgaacccaggggctggtgacggaattttcatagtcaagctatcagagttaagaagaggagcatgtcaaagtacaattagagacaaatatatagtcgcgtggagccaagagcggattcctcagtctcgtaggtctcttgacgaccgttgatctgcttgatctcgtctcccgaaaatgaaaatagactctgctaagctattcttctgcttcgccggagcctgaagggcgtactagggttgcgaggtccaatgcattaatgcattgcagatgagctgtatctggaagaggtaaacccgaaacgcgttttattcttgttgacatggagctattaaatcactagaaggcactctttgctgcttggacaaatgaacgtatcttatcgagatcctgaacaccatttgtctcaactccggagctgacatcgacaccaacgatcttatatccagattcgtcaagctgtttgatgatttcagtaacgttaagtggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggaggcgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatcttgttgccctgctttccgtgcgaaatactaccggtacttttgggaaacaagggaacaggagggcgctgctgtgcgcggttctgagtgttcaggattgaagctgaagaaggtgctgaggaagcgtagaactgttgcggacgcgagttctgagaagagctgtaccgattggtgaaagccgaagaagtgagttggtgccctgttgcctggataatgtttgcaactcgctggttctgcagagacggagacaaatgctggctacgatgttgctgattcaggttgatacctcggtcgagatactgttttggtttgatagggtggatttggttgcagagaagaagaaaggaaggtcaaagagggaaaactgggcggagggaaggattttgtatcaggcagcaaactgccactgcagtggccctggcagtgccgggcgaggcacccacgcacggccgcgcaaccggttggtccttgcccaccacgaaacccttctgaaaggtcagatggaagtgtgcgacagtgcgcgtccccaagccaatgcaggcgccatggatccactccccacccgcaagatttcactgtgcgttcttattggttgccgcaaggccagccaaagggggaagtatgagtcacagcaccgatacaagaaaattgcagaactaacatatggatgcgcgcgctattctgtagagctctgggcaaagcaccaatcctgcgggtcggtacacacactagcactgccccacctgaggcagtcagccccgctgaccgaattgccaagagccaatggagacggaaagccaacgctgatggagcaccatctgaatggacctcgctcgcttgcctggaagggacaagggacaccggagacgcggccgcactagtgcatgcgcaaatttaaagcgctgatatcgatcgcgcgcagatccatatatagggcccgggttataattacctcaggtcgacgtcccatggccattcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaaggtgcacggcccacgtggccactagt

SEQ ID NO: 3 - DNA, 8813 bp, Trichoderma
agactagcggccggtccccttatcccagctgttccacgttggcctgcccctcagttagcgctcaactcaatgcccctcactggcgaggcgagggcaaggatggaggggcagcatcgcctgagttggagcaaagcggccgccatgggagcagcgaaccaacggagggatgccgtgctttgtcgtggctgctgtggccaatccgggcccttggttggctcacagagcgttgctgtgagaccatgagctattattgctaggtacagtatagagagaggagagagagagagagagagagaggggaaaaaaggtgaggttgaagtgagaaaaaaaaaaaaaaaaaaaatccaaccactgacggctgccggctctgccacccccctccctccaccccagaccacctgcacactcagcgcgcagcatcacctaatcttggctcgccttcccgcagctcaggttgttttttttttctctctccctcgtcgaagccgcccttgttcccttatttatttccctctccatccttgtctgcctttggtccatctgcccctttgtctgcatctcttttgcacgcatcgccttatcgtcgtctcttttttcactcacgggagcttgacgaagacctgactcgtgagcctcacctgctgatttctctccccccctcccgaccggcttgacttttgtttctcctccagtaccttatcgcgaagccggaagaaccctctttaaccccatcaaacaagtttgtacaaaaaagcaggctatgcaacagaagcgtcttactgctgccctggtggccgctttggccgctgtggtctctgccgagtcggatgtcaagtccttgaccaaggacaccttcaacgacttcatcaactccaatgacctcgtcctggctgagtgtatgtctctctctctctctctccccccctcccctttgccttctgccctctcaagcttctgcatctctcgacccctcccccgccagccccccggcatcgagatccccgctaacagctgcaatcttccagtcttcgctccctggtgcggccactgcaaggctctcgcccccgagtacgaggaggcggccacgactctcaaggacaagagcatcaagctcgccaaggtcgactgtgtcgaggaggctgacctctgcaaggagcatggagttgagggctaccccacgctcaaggtcttccgtggcctcgataaggtcgctccctacactggtccccgcaaggctgacgggtaagctttgaattgcactgttctttgcatcaatccattcattcgctaacgttggttgtcctttcagcatcacctcctacatggtgaagcagtccctgcctgccgtctccgccctcaccaaggataccctcgaggacttcaagaccgccgacaaggtcgtcctggtcgcctacatcgccgccgatgacaaggcctccaacgagaccttcactgctctggccaacgagctgcgtgacacctacctctttggtggcgtcaacgatgctgccgttgctgaggctgagggcgtcaagttcccttccattgtcctctacaagtccttcgacgagggcaagaacgtcttcagcgagaagttcgatgctgaggccattcgcaactttgctcaggttgccgccactcccctcgttggcgaagttggccctgagacctacgccggctacatgtctgccggtatccctctggcttacatcttcgccgagaccgccgaggagcgtgagaacctggccaagaccctcaagcccgtcgccgagaagtacaagggcaagatcaacttcgccaccatcgacgccaagaactttggctcgcacgccggcaacatcaacctcaagaccgacaagttccccgcctttgccattcacgacattgagaagaacctcaagttcccctttgaccagtccaaggagatcaccgagaaggacattgccgcctttgtcgacggcttctcctctggcaagattgaggccagcatcaagtccgagcccatccccgagacccaggagggccccgtcaccgttgtcgttgcccactcttacaaggacattgtccttgacgacaagaaggacgtcctgattgagttctacgctccctggtgcggtcactgcaaggctctcgcccccaagtacgatgagctcgccagcctgtatgccaagagcgacttcaaggacaaggttgtcatcgccaaggttgatgccactgccaacgacgtccccgacgagatccagggcttccccaccatcaagctctaccccgccggtgacaagaagaaccccgtcacctacagcggtgcccgcactgttgaggacttcatcgagttcatcaaggagaacggcaagtacaaggccggcgtcgagatccccgccgagcccaccgaggaggctgaggcttccgagtccaaggcctctgaggaggccaaggcttccgaggagactcacgatgagctgtaaacccagctttcttgtacaaagtggttcgatggtttaggcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttacctgtggcgccggtgatgccggccacgatgcgtccggcgtagaggatcctctagctagaaagaaggattacctctaaacaagtgtacctgtgcattctgggtaaacgactcataggagagttgtaaaaaagtttcggccggcgtattgggtgttacggagcattcactaggcaaccatggttactattgtatacccatcttagtaggaatgattttcgaggtttatacctacgatgaatgtgtgtcctgtaggcttgagagttcaaggaagaaacagtgcaattatctttgcgaacccaggggctggtgacggaattttcatagtcaagctatcagagttaagaagaggagcatgtcaaagtacaattagagacaaatatatagtcgcgtggagccaagagcggattcctcagtctcgtaggtctcttgacgaccgttgatctgcttgatctcgtctcccgaaaatgaaaatagactctgctaagctattcttctgcttcgccggagcctgaagggcgtactagggttgcgaggtccaatgcattaatgcattgcagatgagctgtatctggaagaggtaaacccgaaacgcgttttattcttgttgacatggagctattaaatcactagaaggcactctttgctgcttggacaaatgaacgtatcttatcgagatcctgaacaccatttgtctcaactccggagctgacatcgacaccaacgatcttatatccagattcgtcaagctgtttgatgatttcagtaacgttaagtggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggaggcgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatcttgttgccctgctttccgtgcgaaatactaccggtacttttgggaaacaagggaacaggagggcgctgctgtgcgcggttctgagtgttcaggattgaagctgaagaaggtgctgaggaagcgtagaactgttgcggacgcgagttctgagaagagctgtaccgattggtgaaagccgaagaagtgagttggtgccctgttgcctggataatgtttgcaactcgctggttctgcagagacggagacaaatgctggctacgatgttgctgattcaggttgatacctcggtcgagatactgttttggtttgatagggtggatttggttgcagagaagaagaaaggaaggtcaaagagggaaaactgggcggagggaaggattttgtatcaggcagcaaactgccactgcagtggccctggcagtgccgggcgaggcacccacgcacggccgcgcaaccggttggtccttgcccaccacgaaacccttctgaaaggtcagatggaagtgtgcgacagtgcgcgtccccaagccaatgcaggcgccatggatccactccccacccgcaagatttcactgtgcgttcttattggttgccgcaaggccagccaaagggggaagtatgagtcacagcaccgatacaagaaaattgcagaactaacatatggatgcgcgcgctattctgtagagctctgggcaaagcaccaatcctgcgggtcggtacacacactagcactgccccacctgaggcagtcagccccgctgaccgaattgccaagagccaatggagacggaaagccaacgctgatggagcaccatctgaatggacctcgctcgcttgcctggaagggacaagggacaccggagacgcggccgcactagtgcatgcgcaaatttaaagcgctgatatcgatcgcgcgcagatccatatatagggcccgggttataattacctcaggtcgacgtcccatggccattcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaaggtgcacggcccacgtggccactagt

SEQ ID NO: 4 - DNA, 1910 bp, Trichoderma
atgaagtcggcgagcaaattgttctttctctccgtgttttccctatgggcgacgccgggcgcatgctcaagctcgtcaagtacatgcactgtacgtcaacccaaccttggcctcgtttcccctttggaagaatgctttgcgctgacagattttgttgatctagttctccccaaacgccatcattgacgatggatgcgtttcgtatgcgactctcgatagactcaatgtcaaggtgaagcctgctatagacgaactcgttcagacgaccgacttcttttcgcactatcgcttgaacctcttcaacaaaaaatgccccttctggaacgacgaagatggcatgtgcggtaacattgcctgcgccgtcgagacgctggacaacgaagaagatattcccgagatatggagggctcacgagcttagcaagctggaaggccctcgagcgaagcatcccggcaagcaagagcagaggcagaaccctgagcgaccgctgcagggagagctgggggaggatgtaggggagagctgcgtggttgaatacgacgacgagtgtgacgacagagactactgcgtctgggacgacgaaggcgcaacgtccaagggggactacatcagcttgttgcgcaaccccgagcgcttcaccggctatggcggtcaaagtgcaaagcaggtgtgggacgccatctactcggagaactgcttcaagaagagctcgtttcccaagtcggccgatctaggcgtctcgcaccgcccaaccgaggcggctgctctggacttcaagcaggtcctggacaccgctggccgccaggctcaactggaacagcagcggcagagcaacccaaacattccctttgttgccaacactggctacgaggtggacgatgagtgtctggagaagcgcgtgttctaccgggtggtgtcgggaatgcacgccagcatcagcgtccacctgtgctgggacttcctgaaccagagcacggggcaatggcagcccaacttggactgctacgagagccgcctgcacaagtttccagaccgcatcagcaacctctacttcaactacgctctcgtgactcgcgccattgcgaagctgggcccgtatgtactgtcaccgcagtacaccttttgcacaggggacccgttgcaagaccaggagacgcgagacaagattgcggccgtcacgaagcacgcggctagcgtcccgcagatctttgacgagggcgtcatgtttgtcaacggcgaaggcccctcgctcaaggaagatttccgcaatcgcttccgcaacatcagccgggtcatggactgcgtcggctgcgacaagtgccgtctctggggcaagatccagaccagcggctacggcacggctttgaagattctgtttgagttcaacgagggccagaagccgccgcccctcaagaggaccgagctggtggccctcttcaacacgtatgccagactcagctcgtcggtggcggccgttgggcgattcagggccatgattgacatgcgcgacaagatggcgtccaagcccgacttcaagcccgaggatctctacacgctcatcgacgaggcggacgaggacatggacgagtttatcaggatgcaaaatcgtgggagccacggagatacgctgggcgagcaggtcggaaacgaatttgcccgcgtcatgatggccgtcaagattgtgctcaagagttggatccgaacgcccaagatgatgtaagtctcttctctcttttttttccccttcttcgagtggcacaaagctcttcattgagatggactaacacaattctagttggcaaattgtctcggaagagacgtcgagattgtatcgcgcttgggtcggtctgcctgcgcgacccagacggtacgcgttcagactgcccaacttgaatagagacgagttgtga

SEQ ID NO: 5 - DNA, 12297 bp, Trichoderma
aagcttactagtacttctcgagctctgtacatgtccggtcgcgacgtacgcgtatcgatggcgccagctgcaggcggccgcctgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccatcacaagtttgtacaaaaaagcaggctccgcggccgcccccttcaccatcatgcacgtcctgtcgactgcggtgctgctcggctccgttgccgttcaaaaggtcctgggaagaccaggatcaagcggtctgtccgacgtcaccaagaggtctgttgacgacttcatcagcaccgagacgcctattgcactgaacaatcttctttgcaatgttggtcctgatggatgccgtgcattcggcacatcagctggtgcggtgattgcatctcccagcacaattgacccggactgtaagttggccttgatgaaccatatcatatatcgccgagaagtggaccgcgtgctgagactgagacagactattacatgtggacgcgagatagcgctcttgtcttcaagaacctcatcgaccgcttcaccgaaacgtacgatgcgggcctgcagcgccgcatcgagcagtacattactgcccaggtcactctccagggcctctctaacccctcgggctccctcgcggacggctctggtctcggcgagcccaagtttgagttgaccctgaagcctttcaccggcaactggggtcgaccgcagcgggatggcccagctctgcgagccattgccttgattggatactcaaagtggctcatcaacaacaactatcagtcgactgtgtccaacgtcatctggcctattgtgcgcaacgacctcaactatgttgcccagtactggtcagtgcttgcttgctcttgaattacgtctttgcttgtgtgtctaatgcctccaccacaggaaccaaaccggctttgacctctgggaagaagtcaatgggagctcattctttactgttgccaaccagcaccgaggtatgaagcaaatcctcgacattcgctgctactgcacatgagcattgttactgaccagctctacagcacttgtcgagggcgccactcttgctgccactcttggccagtcgggaagcgcttattcatctgttgctccccaggttttgtgctttctccaacgattctgggtgtcgtctggtggatacgtcgactccaacagtatgtcttttcactgtttatatgagattggccaatactgatagctcgcctctagtcaacaccaacgagggcaggactggcaaggatgtcaactccgtcctgacttccatccacaccttcgatcccaaccttggctgtgacgcaggcaccttccagccatgcagtgacaaagcgctctccaacctcaaggttgttgtcgactccttccgctccatctacggcgtgaacaagggcattcctgccggtgctgccgtcgccattggccggtatgcagaggatgtgtactacaacggcaacccttggtatcttgctacatttgctgctgccgagcagctgtacgatgccatctacgtctggaagaagacgggctccatcacggtgaccgccacctccctggccttcttccaggagcttgttcctggcgtgacggccgggacctactccagcagctcttcgacctttaccaacatcatcaacgccgtctcgacatacgccgatggcttcctcagcgaggctgccaagtacgtccccgccgacggttcgctggccgagcagtttgaccgcaacagcggcactccgctgtctgcgcttcacctgacgtggtcgtacgcctcgttcttgacagccacggcccgtcgggctggcatcgtgcccccctcgtgggccaacagcagcgctagcacgatcccctcgacgtgctccggcgcgtccgtggtcggatcctactcgcgtcccaccgccacgtcattccctccgtcgcagacgcccaagcctggcgtgccttccggtactccctacacgcccctgccctgcgcgaccccaacctccgtggccgtcaccttccacgagctcgtgtcgacacagtttggccagacggtcaaggtggcgggcaacgccgcggccctgggcaactggagcacgagcgccgccgtggctctggacgccgtcaactatgccgataaccaccccctgtggattgggacggtcaacctcgaggctggagacgtcgtggagtacaagtacatcaatgtgggccaagatggctccgtgacctgggagagtgatcccaaccacacttacacggttcctgcggtggcttgtgtgacgcaggttgtcaaggaggacacctggcagtcggctattggaccagttgctgatctgcacatcgttaacaaggatttggccccagacggcgtccagcgcccaactgttctggccggtggaacttttccgggcacgctgattaccggtcaaaagggcgacaacttccagctgaacgtgattgatgacctgaccgacgatcgcatgttgacccctacttcgatccattggcatggtttcttccagaagggaaccgcctgggccgacggtccggctttcgttacacagtgccctattatcgcagacaactccttcctctacgatttcgacgttcccgaccaggcgggcaccttctggtaccactcacacttgtctacacagtactgcgacggtctgcgcggtgccttcgttgtttacgaccccaacgaccctcacaaggacctttatgatgtcgatgacggtggcacagttatcacattggctgactggtatcacgtcctcgctcagaccgttgtcggagctgctacacccgactctacgctgattaacggcttgggacgcagccagactggccccgccgacgctgagctggccgttatctctgttgaacacaacaagagataccgtttcagactcgtctccatctcgtgcgatcccaacttcacttttagcgtcgacggtcacaacatgacggttatcgaggttgatggcgtgaatacccgccctctcaccgtcgattccattcaaattttcgccggccagcgatactcctttgtgctgaatgccaatcagcccgaggataactactggatccgcgctatgcctaacatcggacgaaacaccactacccttgatggcaagaatgccgctatcctgcgatacaagaacgccagcgttgaggagcccaaaaccgtcggaggacccgcgcagagcccattgaacgaggccgacctgcgacctctggtgcccgctcctgtccctggcaacgcagttcctggtggtgcggacatcaaccaccgcctgaacctgacattcagcaacggcctcttctctatcaataacgcatcatttacaaaccccagcgtccctgccttgttgcagattctttccggcgcacaaaacgctcaggatctgcttcccaccggttcttatatcggcttggagttgggcaaggtcgttgaactcgtgatccctcccttggccgttggtggcccccatccattccacttgcacggccacaacttttgggtcgtccgaagcgctggttctgacgagtataatttcgacgatgcaattttgcgcgacgtggtcagcattggcgcgggaactgacgaggttactatccgttttgtcactgataacccaggcccttggttcctccattgccacatcgactggcacctcgaagccggcctcgccattgttttcgccgaaggcatcaatcaaaccgcagccgccaacccgactccacaggcctgggacgaactctgccccaagtataacggactctccgcttcccagaaagtgaagcccaagaagggaacagccatctaaaagggtgggcgcgccgacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccatggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatcgatcgcgcgcagatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccc

SEQ ID NO: 6 - DNA, 10208 bp, Trichoderma
aagcttactagtacttctcgagctctgtacatgtccggtcgcgacgtacgcgtatcgatggcgccagctgcaggcggccgcctgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccatcacaagtttgtacaaaaaagcaggctccgcggccgcccccttcaccatgcagacctttggagcttttctcgtttccttcctcgccgccagcggcctggccgcggccgctattggaccagttgctgatctgcacatcgttaacaaggatttggccccagacggcgtccagcgcccaactgttctggccggtggaacttttccgggcacgctgattaccggtcaaaagggcgacaacttccagctgaacgtgattgatgacctgaccgacgatcgcatgttgacccctacttcgatccattggcatggtttcttccagaagggaaccgcctgggccgacggtccggctttcgttacacagtgccctattatcgcagacaactccttcctctacgatttcgacgttcccgaccaggcgggcaccttctggtaccactcacacttgtctacacagtactgcgacggtctgcgcggtgccttcgttgtttacgaccccaacgaccctcacaaggacctttatgatgtcgatgacggtggcacagttatcacattggctgactggtatcacgtcctcgctcagaccgttgtcggagctgctacacccgactctacgctgattaacggcttgggacgcagccagactggccccgccgacgctgagctggccgttatctctgttgaacacaacaagagataccgtttcagactcgtctccatctcgtgcgatcccaacttcacttttagcgtcgacggtcacaacatgacggttatcgaggttgatggcgtgaatacccgccctctcaccgtcgattccattcaaattttcgccggccagcgatactcctttgtgctgaatgccaatcagcccgaggataactactggatccgcgctatgcctaacatcggacgaaacaccactacccttgatggcaagaatgccgctatcctgcgatacaagaacgccagcgttgaggagcccaaaaccgtcggaggacccgcgcagagcccattgaacgaggccgacctgcgacctctggtgcccgctcctgtccctggcaacgcagttcctggtggtgcggacatcaaccaccgcctgaacctgacattcagcaacggcctcttctctatcaataacgcatcatttacaaaccccagcgtccctgccttgttgcagattctttccggcgcacaaaacgctcaggatctgcttcccaccggttcttatatcggcttggagttgggcaaggtcgttgaactcgtgatccctcccttggccgttggtggcccccatccattccacttgcacggccacaacttttgggtcgtccgaagcgctggttctgacgagtataatttcgacgatgcaattttgcgcgacgtggtcagcattggcgcgggaactgacgaggttactatccgttttgtcactgataacccaggcccttggttcctccattgccacatcgactggcacctcgaagccggcctcgccattgttttcgccgaaggcatcaatcaaaccgcagccgccaacccgactccacaggcctgggacgaactctgccccaagtataacggactctccgcttcccagaaagtgaagcccaagaagggaacagccatctaaaagggtgggcgcgccgacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccatggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatcgatcgcgcgcagatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccc

SEQ ID NO: 7 - DNA, 10199 bp, Trichoderma
aagcttactagtacttctcgagctctgtacatgtccggtcgcgacgtacgcgtatcgatggcgccagctgcaggcggccgcctgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccatcacaagtttgtacaaaaaagcaggctccgcggccgcccccttcaccatgtatcggaagttggccgtcatctcggccttcttggccacagctcgtgctgctattggaccagttgctgatctgcacatcgttaacaaggatttggccccagacggcgtccagcgcccaactgttctggccggtggaacttttccgggcacgctgattaccggtcaaaagggcgacaacttccagctgaacgtgattgatgacctgaccgacgatcgcatgttgacccctacttcgatccattggcatggtttcttccagaagggaaccgcctgggccgacggtccggctttcgttacacagtgccctattatcgcagacaactccttcctctacgatttcgacgttcccgaccaggcgggcaccttctggtaccactcacacttgtctacacagtactgcgacggtctgcgcggtgccttcgttgtttacgaccccaacgaccctcacaaggacctttatgatgtcgatgacggtggcacagttatcacattggctgactggtatcacgtcctcgctcagaccgttgtcggagctgctacacccgactctacgctgattaacggcttgggacgcagccagactggccccgccgacgctgagctggccgttatctctgttgaacacaacaagagataccgtttcagactcgtctccatctcgtgcgatcccaacttcacttttagcgtcgacggtcacaacatgacggttatcgaggttgatggcgtgaatacccgccctctcaccgtcgattccattcaaattttcgccggccagcgatactcctttgtgctgaatgccaatcagcccgaggataactactggatccgcgctatgcctaacatcggacgaaacaccactacccttgatggcaagaatgccgctatcctgcgatacaagaacgccagcgttgaggagcccaaaaccgtcggaggacccgcgcagagcccattgaacgaggccgacctgcgacctctggtgcccgctcctgtccctggcaacgcagttcctggtggtgcggacatcaaccaccgcctgaacctgacattcagcaacggcctcttctctatcaataacgcatcatttacaaaccccagcgtccctgccttgttgcagattctttccggcgcacaaaacgctcaggatctgcttcccaccggttcttatatcggcttggagttgggcaaggtcgttgaactcgtgatccctcccttggccgttggtggcccccatccattccacttgcacggccacaacttttgggtcgtccgaagcgctggttctgacgagtataatttcgacgatgcaattttgcgcgacgtggtcagcattggcgcgggaactgacgaggttactatccgttttgtcactgataacccaggcccttggttcctccattgccacatcgactggcacctcgaagccggcctcgccattgttttcgccgaaggcatcaatcaaaccgcagccgccaacccgactccacaggcctgggacgaactctgccccaagtataacggactctccgcttcccagaaagtgaagcccaagaagggaacagccatctaaaagggtgggcgcgccgacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccatggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatcgatcgcgcgcagatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccc

SEQ ID NO: 8 - DNA, 9931 bp, Trichoderma reesei
ctgcagccacttgcagtcccgtggaattctcacggtgaatgtaggccttttgtagggtaggaattgtcactcaagcacccccaacctccattacgcctcccccatagagttcccaatcagtgagtcatggcactgttctcaaatagattggggagaagttgacttccgcccagagctgaaggtcgcacaaccgcatgatatagggtcggcaacggcaaaaaagcacgtggctcaccgaaaagcaagatgtttgcgatctaacatccaggaacctggatacatccatcatcacgcacgaccactttgatctgctggtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtgggactttgatggtcatcaaacaaagaacgaagacgcctcttttgcaaagttttgtttcggctacggtgaagaactggatacttgttgtgtcttctgtgtatttttgtggcaacaagaggccagagacaatctattcaaacaccaagcttgctcttttgagctacaagaacctgtggggtatatatctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccatcacaagtttgtacaaaaaagcaggctccgcggccgcccccttcaccatgcagacctttggagcttttctcgtttccttcctcgccgccagcggcctggccgcggccctccccaccgagggtcagaagacggcttccgtcgaggtccagtacaacaagaactacgtcccccacggccctactgctctcttcaaggccaagagaaagtatggcgctcccatcagcgacaacctgaagtctctcgtggctgccaggcaggccaagcaggctctcgccaagcgccagaccggctcggcgcccaaccaccccagtgacagcgccgattcggagtacatcacctccgtctccatcggcactccggctcaggtcctccccctggactttgacaccggctcctccgacctgtgggtctttagctccgagacgcccaagtcttcggccaccggccacgccatctacacgccctccaagtcgtccacctccaagaaggtgtctggcgccagctggtccatcagctacggcgacggcagcagctccagcggcgatgtctacaccgacaaggtcaccatcggaggcttcagcgtcaacacccagggcgtcgagtctgccacccgcgtgtccaccgagttcgtccaggacacggtcatctctggcctcgtcggccttgcctttgacagcggcaaccaggtcaggccgcacccgcagaagacgtggttctccaacgccgccagcagcctggctgagccccttttcactgccgacctgaggcacggacagagtaagtagacactcactggaattcgttcctttcccgatcatcatgaaagcaagtagactgactgaaccaaacaactagacggcagctacaactttggctacatcgacaccagcgtcgccaagggccccgttgcctacacccccgttgacaacagccagggcttctgggagttcactgcctcgggctactctgtcggcggcggcaagctcaaccgcaactccatcgacggcattgccgacaccggcaccaccctgctcctcctcgacgacaacgtcgtcgatgcctactacgccaacgtccagtcggcccagtacgacaaccagcaggagggtgtcgtcttcgactgcgacgaggacctcccttcgttcagcttcggtgttggaagctccaccatcaccatccctggcgatctgctgaacctgactcccctcgaggagggcagctccacctgcttcggtggcctccagagcagctccggcattggcatcaacatctttggtgacgttgccctcaaggctgccctggttgtctttgacctcggcaacgagcgcctgggctgggctcagaaataaaagggtgggcgcgccgacccagctttcttgtacaaagtggtgatcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccatggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagacacgtataacggcacaagtgtctctcaccaaatgggttatatctcaaatgtgatctaaggatggaaagcccagaatatcgatcgcgcgcagatccatatatagggcccgggttataattacctcaggtcgacgtcccatggccattcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcccaagcttactagtacttctcgagctctgtacatgtccggtcgcgacgtacgcgtatcgatggcgccagctgcaggcggccgc

SEQ ID NO: 9 - DNA, 20 bp, Trichoderma reesei
ctgcagccacttgcagtccc

SEQ ID NO: 10 - DNA, 2221 bp, Trichoderma reesei
  misc_feature (166)..(166): n is a, c, g, or t
aagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatnttttgaggcacagaaacccaatagtcaaccgcggactggcatcatgtatcggaagttggccgtcatctcggccttcttggccacagctcgtgctcagtcggcctgcactctccaatcggagactcacccgcctctgacatggcagaaatgctcgtctggtggcacttgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgtatcttcttgtgggctcccagctgactggccaatttaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcaaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaaccgtggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaaagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtacca

SEQ ID NO: 11 - DNA, 51 bp, Trichoderma reesei
atgtatcggaagttggccgtcatctcggccttcttggccacagctcgtgct

SEQ ID NO: 12 - DNA, 1438 bp, Trichoderma reesei
cagtcggcctgcactctccaatcggagactcacccgcctctgacatggcagaaatgctcgtctggtggcacttgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtaagtgacttaccatgaacccctgacgtatcttcttgtgggctcccagctgactggccaatttaaggtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcaaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgatgtgagtttgatggacaaacatgcgcgttgacaaagagtcaagcagctgactgagatgttacagtactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaac

SEQ ID NO: 13 - DNA, 72 bp, Trichoderma reesei
cctcccggcggaaaccgtggcaccaccaccacccgccgcccagccactaccactggaagctctcccggacct

SEQ ID NO: 14 - protein, 513 aa, Trichoderma
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNRGTTTTRRPATTTGSSPGPTQSHYGQCGGIGYSGPTVCASGTTCQVLNPYYSQCL

SEQ ID NO: 15 - DNA, 2267 bp, Trichoderma reesei
atggctcgttcacggagctccctggccctcgggctgggcctgctctgctggatcacgctgctcttcgctcctctggcgtttgtcggaaaggccaatgccgcgagcgacgacgcggacaactacggcactgttatcggaattgtaagtcgactgacggcagcaaccccgccattttcttggtgttgatgctcaggcagccctgctaacacgcttctcctccgcccaggatctcggaactacctacagctgcgtcggtgtgatgcagaagggcaaggttgagattctcgtcaacgaccagggtaaccgaatcactccctcctacgtggcctttaccgacgaggagcgtctggttggcgattccgccaagaaccaggccgccgccaaccccaccaacaccgtctacgatgtcaagtcagttctaccgccctgttggcttctattgtataagtggacaattagctaactgttgtcacaggcgattgattggccgcaaattcgacgagaaggagatccaggccgacatcaagcacttcccctacaaggtcattgagaagaacggcaagcccgtcgtccaggtccaggtcaacggccagaagaagcagttcactcccgaggagatttctgccatgattcttggcaagatgaaggaggttgccgagtcgtacctgggcaagaaggttacccacgccgtcgtcaccgtccctgcctacttcaacgtgagtcttttccccgaaattcctcgaggattccaagagccatctgctaacagcccgataggacaaccagcgacaggccaccaaggacgccggtaccattgccggcttgaacgttctccgaatcgtcaacgaacccaccgctgccgctatcgcctatggtctggacaagaccgacggtgagcgccagatcattgtctacgatctcggtggtggtacctttgatgtttctctcctgtccattgacaatggcgtcttcgaggtcttggctaccgccggtgacacccaccttggtggtgaggactttgaccagcgcattatcaactacctggccaaggcctacaacaagaagaacaacgtcgacatctccaaggacctcaaggccatgggcaagctcaagcgtgaagccgaaaaggccaagcgtaccctctcttcccagatgagcactcgtatcgaaatcgaggccttcttcgagggcaacgacttctccgagactctcacccgggccaagttcgaggagctcaacatggacctcttcaagaagaccctgaagcctgtcgagcaggttctcaaggacgccaacgtcaagaagagcgaggttgacgacatcgttctggtcggcggttccacccgtatccccaaggttcagtctcttatcgaggagtactttaacggcaagaaggcttccaagggtatcaaccccgacgaggctgttgctttcggtgccgccgtccaggccggtgtcctttctggtgaggaaggtaccgatgacattgttctcatggacgtcaaccccctgactctcggtatcgagaccactggcggagtcatgaccaagctcattccccgcaacacccccatccccactcgcaagagccagatcttctcgactgctgccgataaccagcccgtcgtcctgatccaggtcttcgagggtgagcgttccatgaccaaggacaacaacctcctgggcaagttcgagcttaccggcattcctcctgccccccgcggtgtcccccagattgaggtttccttcgagttggatgccaacggtatcctcaaggtctccgctcacgacaagggcaccggcaagcaggagtccatcaccatcaccaacgacaagggccgtctcacccaggaggagattgaccgcatggttgccgaggccgagaagttcgccgaggaggacaaggctacccgtgagcgcatcgaggcccgtaacggtcttgagaactacgccttcagcctgaagaaccaggtcaatgacgaggagggcctcggcggcaagattgacgaggaggacaaggagactgtaagttgaagcgatccatcactgctttctgatgcggacatgtcacactaacacttgaccagattcttgacgccgtcaaggaggctaccgagtggctcgaggagaacggcgccgacgccactaccgaggactttgaggagcagaaggagaagctgtccaacgtcgcctaccccatcacctccaagatgtaccagggtgctggtggctccgaggacgatggcgacttccacgacgaattgtaa

SEQ ID NO: 16 - DNA, 1942 bp, Trichoderma reesei
atgaagttcaacaccgtcgcggccgctgcggctctgctcgctggtgtcgcgtatgccgaggacgtcgaggagtccaaggcagtcccggagcttcccacctttactgtgagtttgccctctctttcatctttggaaaaggacccaaatgttggcgcttggctccagcttggagcaagcttcttggacgacgggatatcatgaaccgctgctgacagttcccaccaatcgcttagcccacctccatcaaggcggacttcctcgagcagttcaccgacgactgggagtcccggtggaagccttcccacgccaagaaggacaccagcggctccgacaaggacgcagaggaggaatgggcctacgtcggcgagtgggcggtcgaggagccctaccagtacaagggcatcaacggcgacaagggcctcgttgtcaagaaccctgccgcgcaccacgccatctcggccaagttccccaagaagattgacaacaagggcaagacgctcgtcgtgcagtacgaggtgaagctccagagtaagtttggcctctgcaactcccccgtgataaccaaagcgagatgtggacattgtgctgacctatacgcttccagagggactggactgcggcggtgcctacatgaagctgctgcgcgacaacaaggctctccaccaggatgagttcagcaacaccaccccctacgtcatcatgtttggccccgacaagtgcggccacaacaaccgggtccacttcatcgtcaaccacaagaaccccaagactggcgagtacgaggagaagcacctcaactcggccccggccgtcaacattgtcaagacgacggagctctacaccctcattgtccaccccaacaacaccttctccatcaagcagaacggtgtcgagaccaaggccggcagccttctcgaggacctgagccctcccatcaaccctcccaaggagattgatgaccccaaggactccaagcccgacgactgggtcgacgaggctcgcattcccgaccccgaggccgtcaagcccgaggactgggacgaggatgcgccctttgagattgtcgacgaggaggccgtcaagcccgaggactggctcgaggacgagcccaccacgatccccgaccccgaggcccagaagcccgaggactgggatgacgaggaggacggcgactggatccctcccaccgtccccaaccccaagtgcgaggacgtctccggttgcggcccctggaccaagcccatggtcaggaaccccaactacaagggcaagtggactgctccttacattgacaaccctgcctacaagggcgtctgggctccccgcaagatcaagaaccccgactactttgaggacaagacgcccgccaactttgagcccatgggagctgtaagtttcgttcctttaccaagaccttcatgacgctcgattgctaaccagtgctcgacagattggcttcgagatctggaccatgaccaacgacatcctctttgacaacatctacattggccactccattgaggatgccgagaagctggccaacgagaccttcttcgtcaagcaccccattgagaaggcgcttgccgaggctgatgagcccaagtttgacgacacccccaagtcgccctctgacctcaagttcctcgacgaccccgtgacctttgtcaaggagaagcttgacctgttcctgaccattgcccagcgcgaccccgttgaggccatcaagtttgttcccgaggtcgccggtggcattgccgccgtcttcgtcaccctgattgccatcattgtcggtctggtcggccttggctcctcatcggccgcccccaagaaggccgccgccactgctaaggagaaggccaaggacgtttccgaggctgttgcaagcggtgccgacaaggtcaagggagaggttaccaagcgaaccacccgcagccagtcgtag

SEQ ID NO: 17 - DNA, 1910 bp, Trichoderma reesei; identical to SEQ ID NO: 4

SEQ ID NO: 18 - DNA, 3027 bp, Trichoderma reesei
atgaagtcaccgaggaaatcaccgttgctgaagctcctcggagccgcctttctcttctccaccaacgttctcgccatctccgctgttctcggagtcgatctgggaaccgagtacatcaaggcggcgctggtgaagcccggcatcccgcttgagattgtgctcacgaaagattcccgacgaaaagaaacctcggccgtcgccttcaagccggcaaagggcgccttaccggagggccagtaccccgaacggagctatggcgccgacgcaatggcactcgccgcacgattccccggcgaagtatacccgaatctgaagcccctgcttggactgccagtgggggatgccattgtccaagaatatgcggccaggcaccctgcgttgaagctacaggcgcaccccacgcggggaactgctgcgttcaagacggagacgctgtctccggaagaggaggcttggatggtggaggagctgttggccatggagcttcagagcatccagaagaacgcagaggttaccgctggcggcgactcttcgatacgctccatcgtgctcaccgtcccgccgttttacaccatcgaggagaagcgagccctgcagatggcagcagagctcgccggcttcaaggtcctgagccttgtcagcgacggactggccgtgggcctcaactatgccaccagtcgccaattcccgaatatcaacgaaggcgccaagccggaataccacttggtctttgacatgggagcgggctccacaactgctacggtcatgaggttccaaagccgtacggttaaggacgtcggcaagttcaacaagacggttcaggagatccaggttctcggcagcggctgggacaggaccctcggaggagactctctcaactcgctaatcatcgatgacatgattgctcagtttgtggaatccaagggtgctcagaagatttcggcaaccgccgagcaggttcagtctcatggccgcgccgttgcaaagctgagcaaggaagccgagcgtctccgacacgtcctcagcgccaaccagaacacccaagccagctttgagggactgtacgaagatgttgacttcaagtacaagatctctcgggctgacttcgagaccatggcaaaggctcatgtcgagcgagtcaacgctgccatcaaggacgctctgaaggccgcgaacctcgagattggcgatctgacttccgtcattcttcacggtggtgcgacccgtactccgtttgtgcgagaggccattgagaaagctcttggttctggcgacaagatccgtaccaatgtcaactctgatgaggcagccgtctttggtgctgctttccgggctgctgagctcagcccaagcttccgtgtgaaggagattaggatttctgagggtgcaaactacgcagctggcattacttggaaggctgcgaacggcaaggtacaccgccaacgactctggactgccccgtcgccgctcggtggcccggccaaggagattacctttacggaacaggaggactttactggtttattctatcaacaagttgacactgaggataagcccgtcaagtcgttctcgactaagaaccttaccgcctctgttgctgctctgaaagaaaagtatcccacttgtgccgatactggcgttcagttcaaggctgccgcgaagctccgtaccgagaacggcgaggttgccatcgtcaaggcctttgtggagtgcgaggctgaagtcgttgagaaggaaggctttgttgacggcgttaagaacctctttggcttcgggaagaaagatcagaagcccctcgccgaaggaggagacaaggacagtgccgatgcgtctgcggattctgaggccgagacggaggaagctagctctgcgacaaagtcctcctcttccaccagcaccaccaagtccggagatgctgccgagtcaacagaggctgcaaaggaagtcaagaagaagcagcttgtttctatccctgtcgaagtcacgttggaaaaggctggaatccctcagcttaccaaggccgagtggaccaaggccaaggatcgactgaaggcattcgccgcctccgacaaggccaggctgcagcgcgaagaggccctgaaccagctcgaagcattcacttacaaggttcgcgaccttgtcgacaacgaagccttcatctccgcgtctaccgaggcggagcgacagacgctctctgaaaaggctagcgaagcaagtgactggctttatgaggagggcgactcggccacgaaagatgactttgttgctaagctcaaggctctgcaagatctcgtggcaccgatccagaaccgcctggacgaggctgagaagcggcctggtctgattagcgatctgagaaacattctcaacaccacaaatgtgtttattgacactgttcgtgggcagattgctgcgtatgatgaatggaaatccacagcttcagccaagtcggctgaatcagccacctcgagtgctgccgccgaggcgacgaccaacgactttgaagggctcgaggatgaggacgacagccccaaagaggctgaggagaagcccgttccagaaaaggtcgtgcccccgctgcacaactctgaggagattgacacgctcgaggttctctacaaggagactctggagtggctgaacaagctcgaacgccaacaggcagatgttcctctcaccgaagagcccgtgcttgttgtcagcgagctggttgccagacgagatgcgcttgacaaggccagcttagacctcgcgctgaagagctacacccaataccagaagaacaagcccaagaagcccaccaagagcaagaaggcgaagaagcaggacaagacgaagagcgccgacaaggctggcccgacgtttgagtttcccgagggcagcgtgcccctctccggcgaggagctggaggagctggtcaagaagtacatgaaggaggaggaggagacccgcaggcaggccgagggcggacaggcagaggagaagccggcggaagatacagagaagtcgagccatgacgagctctaa

SEQ ID NO: 19 - DNA, 1417 bp, Trichoderma reesei
atggtagccagattgtccagcatctacgcctgtgggctcttagcctggacgcacattgtttgcgcctctcagtttagcgacccgatgcaactacagaagcatcttgcacagaatgactatactttaattgcttgtaagtcatgacaatatcgccttctaaagtgtgtcaactcaggtagaaataattgctaatagtagcttacagttgttgctgtaagagttgttcgggtcaatatcttacctagtcaagtcgagactcgaggctgaccttaaggtatcgctaccacttacagcctcaactgtaagttttggccaggccaagtttgaacccatctcccttaagaacaccgaacttaaaaaaagtcaaacggcagagaagccagcaaactcctcttagaagaatggcagacggtccagcaacatgtcgcctccaccgccaccatcgactgtccgtccagccctaaactctgtcaggagatggacgtcgcctcctttcccgctattcggctctaccgccaggatggctcagtaacacgttatcgagggcctcgtcggaccgcaccgtgagttgacactttcttcgaattttggagttaatctctcaaagcatgaagtgactgactgactaccttacctcccaggatcgacgcctttgtgaagcgtgctctcaaaccatccgtgcagaatgttcctgggcagcaacttgccaacttcatcaccaacgacgactatgtattcatcgccaagctgcaaggcgagagcgagagcatcaattctcactacagggattttgcgcaagagtattctgatcgatactcgtttggcatcatcacgagtggctctgtaccctccaatggcgtctggtgctacaacaacgtcgacggaaatcagcacgcggcgacggacttgaacgatccaaatgccttgaagaagcttctcaatctttgcaccgcggaggtcattccccagcttacacgacgcaatgagatgacttatctttccgtatgtcttctgttctccctcctcacttttaaaatgttcagtagaagaagcttgggcttctgaccccttattccagtcaggccgatccttggtctattacttctccaacaatgaagcagaccgcgaagcatacgtcaaagcgctcaaacccatcgcccagcgatacgccgagttcctccagttcgtcaccgtcgactctggcgagtatcccgatatgctgcgcaatctgggcgttcgctccgccggaggcctggcagtgcaaaacgtccacaacggacatattttccccttcagaggagacgctgctgcttcgcctggacaggttgaccagttcattgtggccatctcagaaggtagggcgcagccttgggatgggaggtttgacgagggacaggaggcgcatgatgagctctga

SEQ ID NO: 20 - DNA, 2174 bp, Trichoderma reesei
atgcggctaacatccttcttctctggcctggccgcctttggccttctgtcatctccagcactggcagatgatgaagctgacaacgtccccgcgcccacatacttcgattccgtcatggtgcctcccttgacagaactaacgccagacaacttcgaaaaggaggcaagcaaaaccaagtggcttcttgtgaagcactacaggtactaagcccttcagccatatcacaccactccccgtctgattcaagctgacgcgtagccgctgtctagtccatactgccaccattgtatcagctacgccccgaccttccagacaacctacgaattctactacacatccaagccagaaggagctggcgacacgagcttcaccgacttctacgacttcaagtttgctgccgtgaactgtatcgcctacagcgacctttgcgttgagaatggcgtcaagctataccccactacggttctatacgagaacggcaaagaggtcaaggccgtaacgggtggccagaacatcaccttcctttctgatctcatcgaagaagctttggagaagtcgaagcctggatctcggcccaagtctctcgcattgccccaaccgggcgacaaagagcgccccaaatctgagcccgagacagcatcgaggagcgcaaccgaggagaagaagcccaagaagccggttgccacgccgaacgaagacggagtgtcagtttccttgacggccgaaaacttccagcgcctggtgactatgactcaggatccctggttcatcaagttttacgcgccgtggtgcccccattgccaagacatggcgcctacctgggagcagctggcgaagaacatgaagggcaagctcaacattggagaggtcaactgtgacaaggagtcgcgattgtgcaaagacgttggtgcgcgggcgtttcccactatcctgttcttcaagggtggagagcgctcagagtacgaggggctccgaggcctgggcgactttatcaaatatgccgaaaacgccgtcgacctcgctagcggagtgcctgacgtggacttggcagcattcaaggctctcgagcagaaggaagacgtcatctttgtctacttttacgaccacgccaccacatcggaggacttcaatgccctcgagaggctgcccctgagtctcatcggacatgccaaactggttaagactaaggatccggccatgtacgagcgcttcaagatcacgacatggcccagattcatggtttcgagggagggtcgccctacgtactaccctcccctcacccctaacgcgatgagagatacccaccaagttctggactggatgaggtcggtttggcttccccttgtccccgaactgttggttaccaacgcccgccagatcatggacaacaaaattgttgtgctcggcgtcctgaatcgagaagaccaggaatccttccagagtgctcttcgggagatgaagagcgcagccaacgagtggatggacaggcaaatccaagagttccagttggagcggaagaagctgcgagacgcgaagcaaatgaggatcgaggaagctgaggaccgagacgatgagcgcgccctgcgggccgccaaggcgatccatattgacatgaacaattccggacggagagaagtggcctttgcgtgggttgatggcgtagcgtggcagcgctggattcgaaccacgtatggcattgatgttaaggacggagaaagagtcattatcaacgaccaagatgtaagcctcaagctcacccccatttgtcctccctctacaatattgctttgcgtttcgaacatgaacgactaacaaaaacatttgaacagagccgcaagtactgggacagcaccgtgacgggcaactacatcctcgtcagccgcacgtccatcctggagacgctcgacaaggtcgtctacaccccgcaggccctcaagcccaagctcaccatttcctctttcgagaagatctttttcgacatccgcgtctccttcaccgagcacccctacctgaccctgggctgcatcgttggcatcgcctttggagccttctcctggctgcgtggccgctctcgccgtggacgcggccacttccggctcgaggattccatcagcattagagatttcaaggacgggttccttggtggatctaacggcaacaccaaggccgactga

SEQ ID NO: 21 - DNA, 1578 bp, Trichoderma reesei
atgcatcagcaaaccctcctcgccaccctcgcggcgagtctcgctgctcttccttttgctcaggcgggcttctattcgaagagctctcccgtgctgcaagtagacgccaagtcgtacgaccgcctcatcacaaagtcgaatcatacctctgtaagtatccgtcctcacacactcacctcactcacaacgcgacatcatatctcatacacatccaccccaaaccaccacaaacacaagacatatatcaagctcaaacacatacacatacatacaaacacatacacacacagatacatacacaactctcatatatatgaaccattcattgacatttcccccaagattgtcgaattctacgccccctggtgcggccactgccaaaacctcaagcccgcctacgaaaaggccgcccgcaccctcgacggcctggccaaggtcgccgccgtcgactgcgacgacgacgccaacaaggccctctgcggctccctcggcgtcaagggcttccccaccctcaagatcgtccgccccggcaagaagcccggccgccccgtcgtcgaggactaccagggccagcgcaccgcgggcgccattgccgacgccgtcgtcgccaagatcaacaaccacgtcgtcaagctgacggacaaggacattgatgcctttctggaaaaggacggcgacaagccaaaggccatcttgttcacggaaaagggaactacgagtgcgctgctgaggagccttgctattgattttctcgacgccgtgaccattggccaggtccgcaacaaggaaaaggctgccgtcgacaggttcggcatctcttcgttcccttccttcgtcctcatccccggaggcggcaaggagcccgtcgtctacagcggcgagctcaacaagaaggacatggtcgagttcctcaagcaggtcgccgagcccaaccccgacccggccccctcaaacggcaagtccggcaagaaggcctccaccaaggacaaggccagcagcaaggaggccccccaaaaggccgccgccgccgacgagtcttcgtccgccgcatcctccgagacctcaacggccgccgcgccggagtcgaccctcatcgacatccccgccctgacttccaaggcagagctcgaggagcactgtctccaaccaaagtcccaaacctgcgtcctcgcctttgtgcccgcgtccgcctcggagatgcgcaacaagatcctttctgccgtctcccagctgcacaccaagtacgtccacggaaagcgccacttccccttcttctctgtcgacagcgacgtcgaaggctctgccgccctcaaggaagccctcggcctctcgggcaagattgagctcgttgccctcaacgcccgccgggggtggtggaggcgatacgaggacggtgagttcagcgttcacagcgtcgagtcctggattgacgccgttcgcatgggcgagggcgagaagaagaagcttcccgagggagtcgtcgtcgagaaggcggagccggcggaggaagcaaagtctgagactgaagctgccgcagctgatgaggccactgagaagcctgagcacgatgagctctaa

SEQ ID NO: 22 - DNA, 1167 bp, Trichoderma reesei
atggtcttgatcaagagcctcgtgctcgccgtcctggccagctcggtggctgccaagtcggccgtcatcgacctgattccgtccaactttgacaagcttgtcttctccggaaagcccacgcttgtcgagttttttgctccctggtgcggccactgcaagaaccttgctcccgtgtacgaggagttggcccaggtgtttgagcatgctaaggacaaggtccagattgcaaaggtcgacgccgactcggagcgagacctcggaaagcggttcggcatccagggcttccccacgctcaagttcttcgatggcaagagcaaggagccgcaggagtacaagtcgggccgtgatctggacagcctgaccaagttcatcactgagaagactggtgtcaagcccaagaagaagggcgagctgcccagcagcgtggtgatgctgaacactaggaccttccacgacactgttggaggcgacaagaatgtcctggtagcgttcactgctccttggtgtggccgtaagtgaagcctcgacccccgactgagtcttgattctcgcatatttacctcttgaccagactgcaagaacctcgcccccacttgggaaaaggttgccaatgacttcgcgggtgatgagaacgttgtgattgccaaggtcgatgccgagggcgctgacagcaaggccgtcgccgaagagtacggcgtcactggctaccccaccatcctcttcttccccgctggcaccaagaagcaggttgactaccaaggcggccgatcggagggtgactttgtcaacttcatcaacgagaaggccggcaccttccgaaccgagggcggcgagctgaatgacatcgccggcaccgtggcgcccctcgacaccatcgtggccaacttcctcagcggcaccggcttggccgaggctgctgctgagatcaaggaggctgttgacctgcttacggatgctgcggagaccaagttcgccgagtactacgtccgcgtcttcgacaagctgagcaagaatgagaagtttgttaacaaggagcttgcgagactgcagggcatcctggccaagggtggccttgccccttctaagcgggatgagatccagatcaagatcaacgtcctgcgcaaatttacccccaaggagaacgaggaccagaaggacgagctgtga

SEQ ID NO: 23 - DNA, 1705 bp, Trichoderma reesei
atgcaacagaagcgtcttactgctgccctggtggccgctttggccgctgtggtctctgccgagtcggatgtcaagtccttgaccaaggacaccttcaacgacttcatcaactccaatgacctcgtcctggctgagtgtatgtctctctctctctctctccccccctcccctttgccttctgccctctcaagcttctgcatctctcgacccctcccccgccagccccccggcatcgagatccccgctaacagctgcaatcttccagtcttcgctccctggtgcggccactgcaaggctctcgcccccgagtacgaggaggcggccacgactctcaaggacaagagcatcaagctcgccaaggtcgactgtgtcgaggaggctgacctctgcaaggagcatggagttgagggctaccccacgctcaaggtcttccgtggcctcgataaggtcgctccctacactggtccccgcaaggctgacgggtaagctttgaattgcactgttctttgcatcaatccattcattcgctaacgttggttgtcctttcagcatcacctcctacatggtgaagcagtccctgcctgccgtctccgccctcaccaaggataccctcgaggacttcaagaccgccgacaaggtcgtcctggtcgcctacatcgccgccgatgacaaggcctccaacgagaccttcactgctctggccaacgagctgcgtgacacctacctctttggtggcgtcaacgatgctgccgttgctgaggctgagggcgtcaagttcccttccattgtcctctacaagtccttcgacgagggcaagaacgtcttcagcgagaagttcgatgctgaggccattcgcaactttgctcaggttgccgccactcccctcgttggcgaagttggccctgagacctacgccggctacatgtctgccggtatccctctggcttacatcttcgccgagaccgccgaggagcgtgagaacctggccaagaccctcaagcccgtcgccgagaagtacaagggcaagatcaacttcgccaccatcgacgccaagaactttggctcgcacgccggcaacatcaacctcaagaccgacaagttccccgcctttgccattcacgacattgagaagaacctcaagttcccctttgaccagtccaaggagatcaccgagaaggacattgccgcctttgtcgacggcttctcctctggcaagattgaggccagcatcaagtccgagcccatccccgagacccaggagggccccgtcaccgttgtcgttgcccactcttacaaggacattgtccttgacgacaagaaggacgtcctgattgagttctacgctccctggtgcggtcactgcaaggctctcgcccccaagtacgatgagctcgccagcctgtatgccaagagcgacttcaaggacaaggttgtcatcgccaaggttgatgccactgccaacgacgtccccgacgagatccagggcttccccaccatcaagctctaccccgccggtgacaagaagaaccccgtcacctacagcggtgcccgcactgttgaggacttcatcgagttcatcaaggagaacggcaagtacaaggccggcgtcgagatccccgccgagcccaccgaggaggctgaggcttccgagtccaaggcctctgaggaggccaaggcttccgaggagactcacgatgagctgtaa

SEQ ID NO: 24 - DNA, 982 bp, Trichoderma reesei
atgaaggcagccctgctcctctccgccctggcctcgtgcgccattggcctcgtcgccgccgccgccgaggacttcaagatcgaggtcacccaccccgtcgagtgcgaccgcaagacgcaaaagggcgacaagctgtccatgcactaccgcggcacgctggccaagacgggcgacaagttcgatgccagtgcgtttcttctattccctttccctctttcctcccatttctctcacacaccaatgacggtcctccttttcttttgatctcattgactgacaagttttggtctacctactctaggctacgatcgtaaccagccattcaacttcaagctgggtgctggccaggtgattaaggggttcgtcttgcccacccccccctaacccacccctctcgttcttttatgacgacgacgacgacgacgacgttgggcgacgttgaggctaacggcttgtagatgggatcagggtctccttgacatgtgcattggcgagaagaggtaagacgaaccgaaccaacccaactgcgtcgctcactgcctccttgggcctctatcaggacgcaatgctgaccattacatcaccaattcaggactctcacgatccctcccgagctgggctacggccagcgcaacatgggccccattcccgccggctcaaccctgagtacgtggctcctatcctcccctacctgaactcccaaacccagagtttcacccacgccgcatggaaaaccaggccgcaggctaacaacacacgatgccatacagtctttgagaccgagctcctcgccatcgagggcgtcaaggcccccgagaagaagcccgtccccgagacgcccattgtcgagaagcccgccgaagagacagaggagagcgtcgtcgagaaggccgccgaggcagccgccagcgtggcctccgaggccgtcgacgccgccaagactgtctttgccgacactgacgagggtcacggggagctgtaa

SEQ ID NO: 25 - DNA, 809 bp, Trichoderma reesei
atgctgacctttaggcggctcttcaccaccgccatcgtcctggtggtgggcctgctcttcttcgtcaagacggccgaggccgccaagggccccaagatcacccacaaggtcttcttcgacattgagcacggcgacgagaagctgggccgcatcgtcctgggcctgtacggcaagacggtccccgagacggccgagaacttccgggccctggccaccggcgagaagggcttcggctacgagggctcgaccttccaccgcgtcatcaagcagtttatgattcagggcggcgactttaccaagggcgatggcaccggtggcaagtcgagtaagttgcctttggttcccaaataagcaatcaattgatcaatcaattgggtggcatggcgtttgtcactgcatctggctctggctctggctaaccttgagggctccgtctagtctacggcaacaagttcaaggacgagaacttcaagctgaagcacaccaagaagggcctgctgtccatggccaacgcgggacccgacaccaacggctcccagttcttcatcaccactgttgttacctcgtatgatttccccaccctccttggaagatcctggataagaagtaggaccaatctaacgaacaacttaaacagatggctcgacggccgacacgtcgtcttcggcgaggttctcgagggctacgacattgttgagaagattgaaaacgtccagaccggccccggcgatcgcccagtgaagccggtcaagattgccaagagcggcgagctggaggttccccccgaaggtattcacgtcgagctctaa

SEQ ID NO: 26 - DNA, 1372 bp, Trichoderma reesei
atgatactgcgcgcggcaatcttcgtcttgctggcgctggtatcgctggcggtttgcgccgaggacttttacaaggtatgccgggacgcaatgcctcgaatcaagcacggagcgtgctgacggacacatgacaggttctaggagtcgacaagtctgcgtcagacaagcagctcaagcaggcctatcgccagctctccaagaagttccacccagacaagaacccgtacgccctcctacagctacacgcagtctcgccaaccttctccaatgtgctaatcactctactgcttctagaggcgatgaaacggcgcacgagaaattcgtgctggtgtccgaggcctacgaagttctgagcgattccgagcttcgcaaagtctacgaccgctacggccacgagggcgtcaagtcccaccgtcaaggcggcggcggaggaggaggaggcgaccccttcgacctcttcagcaggttctttggcggccatggccactttgggagaaacagccgcgagccccggggcagcaacattgaggtccgcatcgagatttccctccgcgacttttacaacggcgccacgaccgagttccagtgggagaagcagcacatatgcgaaaagtgcgagggcacgggcagcgcggacggaaaggtcgagacgtgcagcgtctgcggcggacacggggttcggattgtcaagcagcagctcgttcccggcatgttccagcagatgcagatgcgctgcgaccactgtggcggctcgggcaagaccatcaagaacaagtgttccgtctgccacggcagccgagtcgagcgcaagccgacgactgtcagcctgactgtcgagaggggcattgctcgagatgccaaggtggtgtttgagaacgaagccgaccagagccccgactgggttcctggtgatctcattgtcaacctgggcgagaaggccccgtcatacgaagacaaccccgatcgcgtcgacggcaccttcttccggcgcaagggccatgacctgtactggaccgaggttctgtcgctgcgtgaggcctggatgggtggctggacgcgtaacctcacgcacctcgacaagcacgttgtgcgtcttggacgggagcgaggccaggttgttcagagtgggttggtggaaaccattcccggcgaaggcatgcccatatggcacgaagagggagagagcgtctatcacacacacgagtttggaaatctctacgtcacatacgaagtcattttgccggaccagatggacaagaagatggagagcgagttctgggacctgtgggagaagtggcggtccaagaatggtgtggacctgcaaaaggatctcgggcggcctgagccagggcatgaccatgatgagttatga

SEQ ID NO: 27 - DNA, 685 bp, Trichoderma reesei
atggcgcgccgccagcacctcaccgcgacagtcctgctggccgtcgtgctcttcttcagcatcacgtacctcctctcgggctcgtccagctccaatgcggatcgaacgcgcgaggccgtagtggcagagcccaagtcggaattcaaggtggattttgacggcatgccggccaacctgctggagggagagtcaatagcacccaagctggagaatgcgactctcaagtacgtttcccgcatacccgaacctgctcccatgagccaccgaccatggcagtgtttcaaaggataccagttctgacgcttttctgcaattacatagagccgagctcggtcgcgcaacatggaaattcatgcacacaatggtcgcccgcttccccgagaagccctcgcccgaggagcgcaagacgctcgagaccttcatctacctcttcggccggctgtacccctgcggcgactgcgcgaggcacttccggggcctgctggcaaaatatccgccgcagacgagtagccggaatgcggctgccggatggctgtgttttgtgcacaaccaggtcaacgagaggctgaagaagcccatatttgactgcaacaacattggcgacttttacgactgcggctgcggggacgagaagaaggacgggaaggaggaggccaaggttgatggcgaattggtgaaggaatag

SEQ ID NO: 28 - DNA, 3407 bp, Trichoderma reesei
atggtgatgctggtggcgatcgcgctcgcatggctgggatgctcgctgctgcggccggtagatgccatgcgcgcagactatctggcccagctgcggcaggagacggtggacatgttctatcacggatatagcaactacatggagcatgcgtttcccgaagacgaggtgggttccgctgcgatagaagattgttgttggggctgctgctatgttccagctcccggggggtcggattctctcatatagaactagacagctaacgacttgtgccttttccatatgcttagctgcgtcccatatcgtgcactcccctgacgcgagatcgagacaatccggggcgcatcagcctcaacgatgccctcggcaactactctctgaccctcatagacagcctgtctacccttgccatcctggccggcggcccgcagaacggcccttacacgggaccgcaggctctgagcgacttccaggatggcgtggccgagtttgtgcgacactacggagacgggcgatcggggccctccggcgctgggatacgtgccagaggctttgatctcgacagcaaagttcaggtctttgagaccgtcatccggggcgtgggcggtctccttagcgcgcacctgttcgccattggggagctgccgattaccggatacgtgcccaggccggagggagtcgcaggcgatgatcctctggagctggcccctattccgtggcccaatgggttcaggtacgatggccagctgctgaggctcgcgctcgacctctccgagaggctgcttcccgccttctacacgccgacgggcattccgtatcctcgtgtcaatctccgcagcggcatccccttttacgtcaactcgcctctccaccaaaacctgggcgaggcagtggaggagcagagtggccgtcctgaaattaccgagacctgcagcgccggggcgggaagcctggttctcgaatttaccgtcttgagcaggctcacgggagacgccaggtttgaacaagccgccaagcgagcattctgggaggtctggcatcgcaggagcgaaattggcttgatcgggaacggcatcgacgccgagcgcgggctgtggatcggccctcacgcgggcattggcgcgggcatggacagcttctttgaatatgcgctcaagagccatatcctcctctcgggcctcggtatgcccaacgcctccacgtcgcgccgacagagcacaaccagctggctggatccaaactccctgcacccgccgctgccaccagagatgcacacgtcagatgccttcctccaggcatggcatcaggcgcacgcctcggtcaagcggtacctgtacaccgaccggagccacttcccttattactccaacaaccaccgtgccacgggccagccctatgccatgtggatcgacagcctgggcgccttctatccggggctcctcgccctggccggtgaggtggaagaggccattgaggcgaacctcgtctacacagccttgtggacgcggtactctgcgctgcccgaacgctggtccgtccgcgaaggcaacgtcgaggcaggcatcggctggtggcccgggaggcccgagttcatcgagtcgacgtaccacatctaccgtgcaacccgcgacccgtggtatctgcacgttggcgagatggtcctccgcgacattcggcgtcggtgctatgcggagtgcggctgggccgggcttcaggacgtgcagacgggcgagaagcaggaccgcatggagagcttcttcttgggagagacggcaaaatacatgtacctgctgttcgacccagaccatccactcaacaagctggatgccgcctacgtcttcaccacagaaggccatccgcttatcataccaaagagcaaaaggggtagcggctctcacaacagacaggaccgcgctcgcaaagccaagaagagccgagacgtcgcagtctacacctactacgatgaaagcttcacaaactcttgtccggcccctcggccgccttcagagcatcacctgataggctcggccacggcggccaggccagacttgttctccgtctctcgcttcacagacctgtacagaacgcccaacgtacacgggcccctggagaaggtggagatgcgagacaagaagaagggccgggtggttcgatacagggccacctcaaaccacaccatcttcccctggactcttcccccagccatgctgccggagaatggcacctgcgctgctcccccggaacgcatcatatccttgattgagttcccggccaacgacatcaccagtggaatcacgtcgcggttcggcaaccatctatcgtggcagacgcatctggggccaacggtcaacattctagagggactgaggctccagctcgagcaggtgtcggaccctgccacgggagaagacaagtggaggatcacacacattggcaacacgcagctggggcgccacgagacagtcttcttccacgcggaacacgtaaggcatctcaaggacgaggtgttttcctgccgcagaaggagggacgccgtggaaatcgagctcctggtcgacaagccgagcgataccaacaacaacaacacgcttgcctcgtccgatgacgatgtagtggtagatgcaaaagcagaagagcaagacggcatgctagccgacgacgacggcgacacactcaacgcagaaacactctcctccaactccctcttccagtccctcctccgcgccgtctcctccgtcttcgagcccgtctacaccgccatccccgagtccgaccccagcgccggcaccgccaaggtctacagtttcgacgcctacacgtccaccggccccggcgcgtaccccatgccgtccatctcggacacgcccatccccggcaaccccttttacaacttccgcaacccggcctccaacttcccctggtcgaccgtcttcctcgccggccaggcctgcgagggcccgctccccgcgtccgcgccgcgcgagcaccaggtcattgtcatgctccgcggcggctgctccttcagccgcaagctggacaacatccccagcttctcgccccacgacagggcgctgcagctcgtcgttgtcctcgacgaaccgccgccgccgccgccgccgccgccagccagtcagaacagcggcggcgatgacgacgatgaagatgacgaagacgaccacgacgccgtcaacgacaacgaagacgacaggcgcgacgtgacgcggccactgctcgacacggagcagaccacgcccaagggcatgaagcgcctgcacggcatcccaatggtcctcgtccgagccgcgcggggcgactacgagcttttcgggcatgccattggcgtgggcatgaggcgcaagtatcgggttgaaagccaggggcttgtcgtggagaatgcggttgtgctgtga

SEQ ID NO: 29 - DNA, 1221 bp, Trichoderma reesei
atgaggcctctggcactcatatttgccctcatcttgggcctattgctctgcttagcagccccagcaacggcatcgtcatcatcatcacaacactctccccaagcggcatcagacgagtcagatttaatatgtcacacatcaaacccagacgaatgctatccccgggtcttcgtaccaacgcatgagttccagccagtccacgacgaccagcaactcccaaacggcctccatgtccgtctcaacatctggaccggccaaaaggaagccaagatcaacgtccccgatgaggccaaccctgatctcgatggcctgcccgtcgaccaagccgtggttctcgtcgaccaggagcagccagaaattatccagatccccaagggcgcaccaaaatacgacaatgtcggcaagatcaaggaacccgcgcaagaaggagacgcccaaacggaagccattgcttttgcagagacgttcaacatgctcaagaccggcaagtcgccaagcgccgaggagttcgacaacggactggaaggcctggaggagctctcccacgacatctactacgggctcaaaatcacagaggacgcggacgtggtcaaggcgctattctgcttgatgggggctcgcgacggcgacgcctcggagggagccacgccgcgcgaccagcaagcggccgcgatcctcgccggcgccctgtccaacaatccgtcggcactcgccgagatagccaagatctggcctgagcttctggactcgtcgtgtcctcgcgacggcgccaccatctctgaccgtttctaccaagacaccgtctccgttgccgactctccggcaaaggtcaaggccgccgtctcggccatcaacggcctgatcaaggacggcgccatccgaaagcagtttctcgaaaacagcggcatgaagcagctcctctcggtcctgtgccaagagaagccggagtgggcgggagcgcagcggaaagtcgctcagctggtgctggacaccttcctggacgaggacatgggcgcccagcttggccagtggcccaggggcaaggcatcgaacaacggggtgtgtgcggcgccggagacggcgctcgatgacggatgctgggactatcatgcggacaggatggtgaagctgcatgggacgccgtggagcaaggagttgaagcagaggctgggagatgcgcgcaaggcgaacagcaagttgccggatcatggcgagctgtag

SEQ ID NO: 30 - protein, 648 aa, Trichoderma reesei
MARSRSSLALGLGLLCWITLLFAPLAFVGKANAASDDADNYGTVIGIDLGTTYSCVGVMQKGKVEILVNDQGNRITPSYVAFTDEERLVGDSAKNQAAANPTNTVYDVKRLIGRKFDEKEIQADIKHFPYKVIEKNGKPVVQVQVNGQKKQFTPEEISAMILGKMKEVAESYLGKKVTHAVVTVPAYFNDNQRQATKDAGTIAGLNVLRIVNEPTAAAIAYGLDKTDGERQIIVYDLGGGTFDVSLLSIDNGVFEVLATAGDTHLGGEDFDQRIINYLAKAYNKKNNVDISKDLKAMGKLKREAEKAKRTLSSQMSTRIEIEAFFEGNDFSETLTRAKFEELNMDLFKKTLKPVEQVLKDANVKKSEVDDIVLVGGSTRIPKVQSLIEEYFNGKKASKGINPDEAVAFGAAVQAGVLSGEEGTDDIVLMDVNPLTLGIETTGGVMTKLIPRNTPIPTRKSQIFSTAADNQPVVLIQVFEGERSMTKDNNLLGKFELTGIPPAPRGVPQIEVSGTGKQESITITNDKGRLTQEEIDRMVAEAEKFAEEDKATRERIEARNGLENYAFSLKNQVNDEEGLGGKIDEEDKETILDAVKEATEWLEENGADATTEDFEEQKEKLSNVAYPITSKMYQGAGGSEDDGDFHDEL

SEQ ID NO: 31 - protein, 558 aa, Trichoderma reesei
MKFNTVAAAAALLAGVAYAEDVEESKAVPELPTFTPTSIKADFLEQFTDDWESRWKPSHAKKDTSGSDKDAEEEWAYVGEWAVEEPYQYKGINGDKGLVVKNPAAHHAISAKFPKKIDNKGKTLVVQYEVKLQKGLDCGGAYMKLLRDNKALHQDEFSNTTPYVIMFGPDKCGHNNRVHFIVNHKNPKTGEYEEKHLNSAPAVNIVKTTELYTLIVHPNNTFSIKQNGVETKAGSLLEDLSPPINPPKEIDDPKDSKPDDWVDEARIPDPEAVKPEDWDEDAPFEIVDEEAVKPEDWLEDEPTTIPDPEAQKPEDWDDEEDGDWIPPTVPNPKCEDVSGCGPWTKPMVRNPNYKGKWTAPYIDNPAYKGVWAPRKIKNPDYFEDKTPANFEPMGAIGFEIWTMTNDILFDNIYIGHSIEDAEKLANETFFVKHPIEKALAEADEPKFDDTPKSPSDLKFLDDPVTFVKEKLDLFLTIAQRDPVEAIKFVPEVAGGIAAVFVTLIAIIVGLVGLGSSSAAPKKAAATAKEKAKDVSEAVASGADKVKGEVTKRTTRSQS

SEQ ID NO: 32 - protein, 585 aa, Trichoderma reesei
MKSASKLFFLSVFSLWATPGACSSSSSTCTFSPNAIIDDGCVSYATLDRLNVKVKPAIDELVQTTDFFSHYRLNLFNKKCPFWNDEDGMCGNIACAVETLDNEEDIPEIWRAHELSKLEGPRAKHPGKQEQRQNPERPLQGELGEDVGESCVVEYDDECDDRDYCVWDDEGATSKGDYISLLRNPERFTGYGGQSAKQVWDAIYSENCFKKSSFPKSADLGVSHRPTEAAALDFKQVLDTAGRQAQLEQQRQSNPNIPFVANTGYEVDDECLEKRVFYRVVSGMHASISVHLCWDFLNQSTGQWQPNLDCYESRLHKFPDRISNLYFNYALVTRAIAKLGPYVLSPQYTFCTGDPLQDQETRDKIAAVTKHAASVPQIFDEGVMFVNGEGPSLKEDFRNRFRNISRVMDCVGCDKCRLWGKIQTSGYGTALKILFEFNEGQKPPPLKRTELVALFNTYARLSSSVAAVGRFRAMIDMRDKMASKPDFKPEDLYTLIDEADEDMDEFIRMQNRGSHGDTLGEQVGNEFARVMMAVKIVLKSWIRTPKMIWQIVSEETSRLYRAWVGLPARPRRYAFRLPNLNRDEL

SEQ ID NO: 33 - protein, 1008 aa, Trichoderma reesei
MKSPRKSPLLKLLGAAFLFSTNVLAISAVLGVDLGTEYIKAALVKPGIPLEIVLTKDSRRKETSAVAFKPAKGALPEGQYPERSYGADAMALAARFPGEVYPNLKPLLGLPVGDAIVQEYAARHPALKLQAHPTRGTAAFKTETLSPEEEAWMVEELLAMELQSIQKNAEVTAGGDSSIRSIVLTVPPFYTIEEKRALQMAAELAGFKVLSLVSDGLAVGLNYATSRQFPNINEGAKPEYHLVFDMGAGSTTATVMRFQSRTVKDVGKFNKTVQEIQVLGSGWDRTLGGDSLNSLIIDDMIAQFVESKGAQKISATAEQVQSHGRAVAKLSKEAERLRHVLSANQNTQASFEGLYEDVDFKYKISRADFETMAKAHVERVNAAIKDALKAANLEIGDLTSVILHGGATRTPFVREAIEKALGSGDKIRTNVNSDEAAVFGAAFRAAELSPSFRVKEIRISEGANYAAGITWKAANGKVHRQRLWTAPSPLGGPAKEITFTEQEDFTGLFYQQVDTEDKPVKSFSTKNLTASVAALKEKYPTCADTGVQFKAAAKLRTENGEVAIVKAFVECEAEVVEKEGFVDGVKNLFGFGKKDQKPLAEGGDKDSADASADSEAETEEASSATKSSSSTSTTKSGDAAESTEAAKEVKKKQLVSIPVEVTLEKAGIPQLTKAEWTKAKDRLKAFAASDKARLQREEALNQLEAFTYKVRDLVDNEAFISASTEAERQTLSEKASEASDWLYEEGDSATKDDFVAKLKALQDLVAPIQNRLDEAEKRPGLISDLRNILNTTNVFIDTVRGQIAAYDEWKSTASAKSAESATSSAAAEATTNDFEGLEDEDDSPKEAEEKPVPEKVVPPLHNSEEIDTLEVLYKETLEWLNKLERQQADVPLTEEPVLVVSELVARRDALDKASLDLALKSYTQYQKNKPKKPTKSKKAKKQDKTKSADKAGPTFEFPEGSVPLSGEELEELVKKYMKEEEETRRQAEGGQAEEKPAEDTEKSSHDEL

SEQ ID NO: 34 - protein, 363 aa, Trichoderma reesei
MVARLSSIYACGLLAWTHIVCASQFSDPMQLQKHLAQNDYTLIAFVASRLEADLKVSLPLTASTSNGREASKLLLEEWQTVQQHVASTATIDCPSSPKLCQEMDVASFPAIRLYRQDGSVTRYRGPRRTAPIDAFVKRALKPSVQNVPGQQLANFITNDDYVFIAKLQGESESINSHYRDFAQEYSDRYSFGIITSGSVPSNGVWCYNNVDGNQHAATDLNDPNALKKLLNLCTAEVIPQLTRRNEMTYLSSGRSLVYYFSNNEADREAYVKALKPIAQRYAEFLQFVTVDSGEYPDMLRNLGVRSAGGLAVQNVHNGHIFPFRGDAAASPGQVDQFIVAISEGRAQPWDGRFDEGQEAHDEL

SEQ ID NO: 35 - protein, 688 aa, Trichoderma reesei
MRLTSFFSGLAAFGLLSSPALADDEADNVPAPTYFDSVMVPPLTELTPDNFEKEASKTKWLLVKHYSPYCHHCISYAPTFQTTYEFYYTSKPEGAGDTSFTDFYDFKFAAVNCIAYSDLCVENGVKLYPTTVLYENGKEVKAVTGGQNITFLSDLIEEALEKSKPGSRPKSLALPQPGDKERPKSEPETASRSATEEKKPKKPVATPNEDGVSVSLTAENFQRLVTMTQDPWFIKFYAPWCPHCQDMAPTWEQLAKNMKGKLNIGEVNCDKESRLCKDVGARAFPTILFFKGGERSEYEGLRGLGDFIKYAENAVDLASGVPDVDLAAFKALEQKEDVIFVYFYDHATTSEDFNALERLPLSLIGHAKLVKTKDPAMYERFKITTWPRFMVSREGRPTYYPPLTPNAMRDTHQVLDWMRSVWLPLVPELLVTNARQIMDNKIVVLGVLNREDQESFQSALREMKSAANEWMDRQIQEFQLERKKLRDAKQMRIEEAEDRDDERALRAAKAIHIDMNNSGRREVAFAWVDGVAWQRWIRTTYGIDVKDGERVIINDQDVSLKLTPICPPSTILLCSRKYWDSTVTGNYILVSRTSILETLDKVVYTPQALKPKLTISSFEKIFFDIRVSFTEHPYLTLGCIVGIAFGAFSWLRGRSRRGRGHFRLEDSISIRDFKDGFLGGSNGNTKAD

SEQ ID NO: 36 - protein, 461 aa, Trichoderma reesei
MHQQTLLATLAASLAALPFAQAGFYSKSSPVLQVDAKSYDRLITKSNHTSIVEFYAPWCGHCQNLKPAYEKAARTLDGLAKVAAVDCDDDANKALCGSLGVKGFPTLKIVRPGKKPGRPVVEDYQGQRTAGAIADAVVAKINNHVVKLTDKDIDAFLEKDGDKPKAILFTEKGTTSALLRSLAIDFLDAVTIGQVRNKEKAAVDRFGISSFPSFVLIPGGGKEPVVYSGELNKKDMVEFLKQVAEPNPDPAPSNGKSGKKASTKDKASSKEAPQKAAAADESSSAASSETSTAAAPESTLIDIPALTSKAELEEHCLQPKSQTCVLAFVPASASEMRNKILSAVSQLHTKYVHGKRHFPFFSVDSDVEGSAALKEALGLSGKIELVALNARRGWWRRYEDGEFSVHSVESWIDAVRMGEGEKKKLPEGVVVEKAEPAEEAKSETEAAAADEATEKPEHDEL

SEQ ID NO: 37 - protein, 368 aa, Trichoderma reesei
MVLIKSLVLAVLASSVAAKSAVIDLIPSNFDKLVFSGKPTLVEFFAPWCGHCKNLAPVYEELAQVFEHAKDKVQIAKVDADSERDLGKRFGIQGFPTLKFFDGKSKEPQEYKSGRDLDSLTKFITEKTGVKPKKKGELPSSVVMLNTRTFHDTVGGDKNVLVAFTAPWCGHCKNLAPTWEKVANDFAGDENVVIAKVDAEGADSKAVAEEYGVTGYPTILFFPAGTKKQVDYQGGRSEGDFVNFINEKAGTFRTEGGELNDIAGTVAPLDTIVANFLSGTGLAEAAAEIKEAVDLLTDAAETKFAEYYVRVFDKLSKNEKFVNKELARLQGILAKGGLAPSKRDEIQIKINVLRKFTPKENEDQKDEL

SEQ ID NO: 38 - protein, 502 aa, Trichoderma reesei
MQQKRLTAALVAALAAVVSAESDVKSLTKDTFNDFINSNDLVLAEFFAPWCGHCKALAPEYEEAATTLKDKSIKLAKVDCVEEADLCKEHGVEGYPTLKVFRGLDKVAPYTGPRKADGITSYMVKQSLPAVSALTKDTLEDFKTADKVVLVAYIAADDKASNETFTALANELRDTYLFGGVNDAAVAEAEGVKFPSIVLYKSFDEGKNVFSEKFDAEAIRNFAQVAATPLVGEVGPETYAGYMSAGIPLAYIFAETAEERENLAKTLKPVAEKYKGKINFATIDAKNFGSHAGNINLKTDKFPAFAIHDIEKNLKFPFDQSKEITEKDIAAFVDGFSSGKIEASIKSEPIPETQEGPVTVVVAHSYKDIVLDDKKDVLIEFYAPWCGHCKALAPKYDELASLYAKSDFKDKVVIAKVDATANDVPDEIQGFPTIKLYPAGDKKNPVTYSGARTVEDFIEFIKENGKYKAGVEIPAEPTEEAEASESKASEEAKASEETHDEL

SEQ ID NO: 39 - protein, 190 aa, Trichoderma reesei
MKAALLLSALASCAIGLVAAAAEDFKIEVTHPVECDRKTQKGDKLSMHYRGTLAKTGDKFDASYDRNQPFNFKLGAGQVIKGWDQGLLDMCIGEKRTLTIPPELGYGQRNMGPIPAGSTLIFETELLAIEGVKAPEKKPVPETPIVEKPAEETEESVVEKAAEAAASVASEAVDAAKTVFADTDEGHGEL

SEQ ID NO: 40 - protein, 207 aa, Trichoderma reesei
MLTFRRLFTTAIVLVVGLLFFVKTAEAAKGPKITHKVFFDIEHGDEKLGRIVLGLYGKTVPETAENFRALATGEKGFGYEGSTFHRVIKQFMIQGGDFTKGDGTGGKSIYGNKFKDENFKLKHTKKGLLSMANAGPDTNGSQFFITTVVTSWLDGRHVVFGEVLEGYDIVEKIENVQTGPGDRPVKPVKIAKSGELEVPPEGIHVEL

SEQ ID NO: 41 - protein, 413 aa, Trichoderma reesei
MILRAAIFVLLALVSLAVCAEDFYKVLGVDKSASDKQLKQAYRQLSKKFHPDKNPGDETAHEKFVLVSEAYEVLSDSELRKVYDRYGHEGVKSHRQGGGGGGGGDPFDLFSRFFGGHGHFGRNSREPRGSNIEVRIEISLRDFYNGATTEFQWEKQHICEKCEGTGSADGKVETCSVCGGHGVRIVKQQLVPGMFQQMQMRCDHCGGSGKTIKNKCSVCHGSRVERKPTTVSLTVERGIARDAKVVFENEADQSPDWVPGDLIVNLGEKAPSYEDNPDRVDGTFFRRKGHDLYWTEVLSLREAWMGGWTRNLTHLDKHVVRLGRERGQVVQSGLVETIPGEGMPIWHEEGESVYHTHEFGNLYVTYEVILPDQMDKKMESEFWDLWEKWRSKNGVDLQKDLGRPEPGHDHDEL

SEQ ID NO: 42 - protein, 182 aa, Trichoderma reesei
MARRQHLTATVLLAVVLFFSITYLLSGSSSSNADRTREAVVAEPKSEFKVDFDGMPANLLEGESIAPKLENATLKAELGRATWKFMHTMVARFPEKPSPEERKTLETFIYLFGRLYPCGDCARHFRGLLAKYPPQTSSRNAAAGWLCFVHNQVNERLKKPIFDCNNIGDFYDCGCGDEKKDG

SEQ ID NO: 43 - protein, 1070 aa, Trichoderma reesei
MVMLVAIALAWLGCSLLRPVDAMRADYLAQLRQETVDMFYHGYSNYMEHAFPEDELRPISCTPLTRDRDNPGRISLNDALGNYSLTLIDSLSTLAILAGGPQNGPYTGPQALSDFQDGVAEFVRHYGDGRSGPSGAGIRARGFDLDSKVQVFETVIRGVGGLLSAHLFAIGELPITGYVPRPEGVAGDDPLELAPIPWPNGFRYDGQLLRLALDLSERLLPAFYTPTGIPYPRVNLRSGIPFYVNSPLHQNLGEAVEEQSGRPEITETCSAGAGSLVLEFTVLSRLTGDARFEQAAKRAFWEVWHRRSEIGLIGNGIDAERGLWIGPHAGIGAGMDSFFEYALKSHILLSGLGMPNASTSRRQSTTSWLDPNSLHPPLPPEMHTSDAFLQAWHQAHASVKRYLYTDRSHFPYYSNNHRATGQPYAMWIDSLGAFYPGLLALAGEVEEAIEANLVYTALWTRYSALPERWSVREGNVEAGIGWWPGRPEFIESTYHIYRATRDPWYLHVGEMVLRDIRRRCYAECGWAGLQDVQTGEKQDRMESFFLGETAKYMYLLFDPDHPLNKLDAAYVFTTEGHPLIIPKSKRGSGSHNRQDRARKAKKSRDVAVYTYYDESFTNSCPAPRPPSEHHLIGSATAARPDLFSVSRFTDLYRTPNVHGPLEKVEMRDKKKGRVVRYRATSNHTIFPWTLPPAMLPENGTCAAPPERIISLIEFPANDITSGITSRFGNHLSWQTHLGPTVNILEGLRLQLEQVSDPATGEDKWRITHIGNTQLGRHETVFFHAEHVRHLKDEVFSCRRRRDAVEIELLVDKPSDTNNNNTLASSDDDVVVDAKAEEQDGMLADDDGDTLNAETLSSNSLFQSLLRAVSSVFEPVYTAIPESDPSAGTAKVYSFDAYTSTGPGAYPMPSISDTPIPGNPFYNFRNPASNFPWSTVFLAGQACEGPLPASAPREHQVIVMLRGGCSFSRKLDNIPSFSPHDRALQLVVVLDEPPPPPPPPPANDRRDVTRPLLDTEQTTPKGMKRLHGIPMVLVRAARGDYELFGHAIGVGMRRKYRVESQGLVVENAVVL

SEQ ID NO: 44 - protein, 406 aa, Trichoderma reesei
MRPLALIFALILGLLLCLAAPATASSSSSQHSPQAASDESDLICHTSNPDECYPRVFVPTHEFQPVHDDQQLPNGLHVRLNIWTGQKEAKINVPDEANPDLDGLPVDQAVVLVDQEQPEIIQIPKGAPKYDNVGKIKEPAQEGDAQTEAIAFAETFNMLKTGKSPSAEEFDNGLEGLEELSHDIYYGLKITEDADVVKALFCLMGARDGDASEGATPRDQQAAAILAGALSNNPSALAEIAKIWPELLDSSCPRDGATISDRFYQDTVSVADSPAKVKAAVSAINGLIKDGAIRKQFLENSGMKQLLSVLCQEKPEWAGAQRKVAQLVLDTFLDEDMGAQLGQWPRGKASNNGVCAAPETALDDGCWDYHADRMVKLHGTPWSKELKQRLGDARKANSKLPDHGEL

SEQ ID NO: 45 - protein, 505 aa, Trichoderma
AIGPVADLHIVNKDLAPDGVQRPTVLAGGTFPGTLITGQKGDNFQLNVIDDLTDDRMLTPTSIHWHGFFQKGTAWADGPAFVTQCPIIADNSFLYDFDVPDQAGTFWYHSHLSTQYCDGLRGAFVVYDPNDPHKDLYDVDDGGTVITLADWYHVLAQTVVGAATPDSTLINGLGRSQTGPADAELAVISVEHNKRYRFRLVSISCDPNFTFSVDGHNMTVIEVDGVNTRPLTVDSIQIFAGQRYSFVLNANQPEDNYWIRAMPNIGRNTTTLDGKNAAILRYKNASVEEPKTVGGPAQSPLNEADLRPLVPAPVPGNAVPGGADINHRLNLTFSNGLFSINNASFTNPSVPALLQILSGAQNAQDLLPTGSYIGLELGKVVELVIPPLAVGGPHPFHLHGHNFWVVRSAGSDEYNFDDAILRDVVSIGAGTDEVTIRFVTDNPGPWFLHCHIDWHLEAGLAIVFAEGINQTAAANPTPQAWDELCPKYNGLSASQKVKPKKGTAI